SEQUENCE LISTING (79 sequences)

SEQ ID NO: 1 - protein, 647 aa, Eremothecium gossypii
MAQQLLKQVLRTLALPVIMPLLALNRRFRILDDIRTITYFVQALVAYGWCTLTQRFPTWYVFEAQVAKHGDSPCIRYCRPQARKGDFTVETYTYRETYEHVLRLSYVLYHDYGVRAGEHVAVNYANKPMFLFLWLALWNIGAVPAFVNHNQKGTPLIHSVKISNARLLFVDAGTTNLPKGAELLKELPELQIHHFDEEQMLAIIKSDKSPSLLIKRGERTPRTLHDYDPAMLIYTSGTTGLPKSAIMSWRKATLGCSLFGFMMRISPESVVLTAMPLYHSTAALLGVCAVFTQGGCIAISNKFSTTTFWKEAYLSKATHIQYVGEVCRYLMNAPKSEYEDMATVKVAYGNGLRQSIWMDFKKRFRIEAIGEFYASTEAPFATTAFQLGTFGVGACRSYGSLVHWILSYQQTLVRVDPDDESVVYRNENGFCEVPASDEPGELLMRIFFPRKPHTSFQGYLGNKKATESKVLRDVFRKGDAWYRSGDLLKSDKYGQWYFVDRMGDTYRWKSENVSTTEVENQLLSFNKDLFDCLVVVGLKPSYEGRAGFAVIQLNPARRGLDHASLLDDLVEYLKHALPRYALPLFIKFTNQLETTDNYKFAKKQYKNQQLPHGADGDETIYWLKDYSQYKVLTDEDWEQISTGKAKL

SEQ ID NO: 2 - DNA, 1953 bp, Eremothecium gossypii
atggcacagcaactactgaagcaagtgttgcggacccttgcgcttccggtgataatgccgcttttagcgctgaacaggaggtttcggatattggacgatattcggacaatcacgtacttcgttcaggcgttggtagcatacggatggtgcacactgacacaacggttcccaacatggtatgttttcgaggcccaggttgcgaaacatggggattcgccgtgtatccgatactgccgcccgcaggcgcggaagggcgacttcacggtggagacgtacacttaccgtgagacgtacgagcatgtgctgcggctgtcttacgtgctataccacgactacggggtgcgtgccggcgagcatgtggcggttaactatgcgaacaagccgatgttcctgttcctatggctggcgctgtggaacatcggcgctgtgccggcatttgtaaaccacaaccagaagggcacgccgctgattcactcggtgaagatttcgaacgcgcgcttgttatttgtggatgcaggaacgaccaacctgcccaaagggtccgaggcggagcttttgaaggagcttcctgagttacagatccaccactttgacgaggagcagatgttggcgattattaaaagcgacaagagcccctctctcctaatcaaacgtggcgagcgcacgccgcgcaccctccacgactatgatcctgcgatgttgatctacacgtcggggacgacgggactgcctaaatcggcaattatgtcctggcgtaaagctactcttggatgctcgctgtttggttttatgatgcgtataagcccagaaagtgtggtactcacggccatgccgctgtatcactcgaccgcggcgttgctgggtgtctgcgcagtttttacacagggcggctgtattgccatctccaataagttttcgactaccacattctggaaggaggcatatctgtccaaggccacgcatatccagtatgtgggagaagtgtgtcggtacctcatgaacgctccgaaatccgagtacgaagatatggcaacagttaaggtcgcttacgggaacggactccgccaaagtatctggatggatttcaaaaagaggttccgcattgaggcgatcggtgaattctacgcatcaactgaggcgccatttgccacaactgccttccaactgggtacgtttggcgttggagcatgcaggagctatggcagccttgtgcactggatactgtcgtaccagcaaactctagtgcgggttgatccggacgatgagtcggtagtataccggaacgaaaatggattctgcgaggtcccagcaagtgatgaacctggtgaactactaatgcggatctttttcccccgcaaaccgcacacctcgttccaggggtacttgggcaacaaaaaggcgacggagagcaaggttctacgcgatgttttcaggaagggagatgcatggtaccggtcaggcgatctcttgaaatccgacaagtacgggcaatggtacttcgtggaccggatgggtgatacgtaccggtggaaatccgaaaatgtctcgactaccgaggtggagaatcagttgctctcgttcaacaaggacctctttgactgtttggttgtagtgggcctgaagattccaagctacgagggtagagccgggtttgctgttatccaactgaatccagcgcgccgcggactggaccatgccagtttgttagacgaccttgtcgagtatttgaaacatgctcttcctcggtacgccttgccgctgttcatcaagttcacaaaccagctggaaacaaccgataactataagttcgccaagaaacagtacaaaaaccagcagttgcctcatggtgcggatggggacgagacaatttactggttaaaagactactcgcagtacaaagtcttgaccgacgaggactgggagcagatatcaaccggaaaggcaaagctttag

SEQ ID NO: 3 - protein, 626 aa, Eremothecium gossypii
MVTKVVDGKETEVEKTWLYYEMGPYQYVTYDQLHVEMHDYGRGMVKMGLQPGGEDRLHIFGATSHRWMRTFLAAQSQAITVVTAYDTLGESGLIYSLQQTGSKAIFVDNNLLEKLVKPVQEIPDLKYVIHADPLDPEDKRYGGRMYSDAQKAIDRMKEVRPDIEVYSMDEVVELGSLCRDSIFVHRPRKKDLACIMYTSGSTGDPKGVSLTHANIVAGIGGVSVVINRAIVKPDDRVIAFLPLAHIFELVFELTCLYWGALIGYGSVKTLSEASVRNCKGDMKEFRPSVMVGVAAVWEGVRKAIVAQVTKLPPFKQKIFWAAYHTKLRMKKCHIPGGDLIGSMIFKKVRETTGGNLRYILNGGSPLSRDTQVFISNLICPVLIGYGLTETVANGCIVPPHHFKYGVVGDILGSLTVKLVDVEELGYLAKNNQGELWVKGPAVFKDYLQNEAETAAALEDGWFKTGDIAEWTKKGQLRLIDRKKNLVKTLNGEYIALEKLECIYRSNKYVANICVYADQTKVKPIAIVVPNVNAVTDLAISLGLIKDGCEVRDVYDSKKLKKVILDDMHKTAKGQGLGGIELILGFVIFDDEWTPQNGYVTSAQKLQRRKILSAVQSEVDALYAANS

SEQ ID NO: 4 - DNA, 2094 bp, Eremothecium gossypii
atgaagtcggccagtgttatagtaggagagcccgcagggcctcacgagacggcgccacggcgcaactccaagtgcccggatgcggtcgtggagcggccgctggggttcagctgcaacacggtatatgagtttgcgttggaggcgatggagcgcggcgggcggcagcgcgcgatggggtggcgggagacggtggagatccacgaggaccgcaagatggtgacgaaggtggtggacggcaaggagacggaggtggagaagacgtggttgtactacgagatgggcccgtaccagtacgtgacgtacgaccagctgcacgtggagatgcacgactacgggcgggggatggtgaagatggggctccagccgggcggcgaggaccgcttgcacattttcggcgcgacgtcgcaccggtggatgcggacgttcttggcagcgcagtcgcaggccatcacggtggtgacggcatacgacacgttgggcgagagcggcttgatctactcgctccagcagacggggtcgaaggcgatcttcgtggacaacaacctcttggagaagttggtgaagccggtgcaggagatcccggacttgaagtacgtgatccacgcggacccgctcgacccggaggacaagcgctacggcggccggatgtactctgacgcgcagaaggcgatcgaccgcatgaaggaggttcggccggacatcgaggtttacagcatggacgaggtcgtggagctcggatcgctctgccgggactcgatttttgtgcaccggccacgcaagaaggaccttgcgtgcatcatgtacacctcgggctcgacaggtgacccgaagggtgtgtcgttgacccacgctaacatcgtggcgggcattggcggtgtttccgttgtgatcaaccgcgcgattgtgaagcctgacgatcgtgtcatcgcgttcttgccgcttgcgcatatttttgagcttgtgttcgagttgacctgtctctactggggcgccttaattggctacggctccgtcaagacgttgagcgaggcttcggtccgcaactgtaagggcgacatgaaggagttccggccgtccgtcatggtcggtgtcgcagctgtctgggagggtgtcaggaaggctattgttgcgcaggtcactaagttgcctccgttcaagcaaaagatattctgggcggcctaccacaccaagctacgcatgaagaagtgccacattccaggcggcgatctaataggaagcatgatctttaagaaggtgcgtgagaccactggtggcaaccttcgctacatcttgaatggtggctctccattgtcgcgggatacgcaagtttttatttccaacttgatttgccccgtgttgattggttacggcttaacggagactgtggcgaatggctgtatagtgcctccacaccacttcaagtacggggttgtgggagacattcttggttctctaacggtcaaattggtcgatgtcgaggagctcggctatcttgccaagaacaaccagggtgagctctgggtcaagggccccgccgtgtttaaagactacttgcagaacgaggccgagaccgctgccgctttggaagacgggtggttcaagactggtgacattgccgaatggacgaagaagggtcaattgcgtttgatcgaccgtaagaagaaccttgtcaagacgttgaatggtgaatacatcgctttggagaagttggagtgcatctacagatccaacaagtatgtggccaacatctgtgtctatgctgaccagaccaaggtcaagccaattgcgattgtggttccaaacgtcaatgctgtcaccgatttggccatctcattggggttgatcaaggacggttgcgaggtacgtgatgtttatgatagcaaaaagttgaagaaggtgatcttggacgacatgcataaaactgccaagggccagggattgggtggtattgagttgattcttgggttcgtgatcttcgatgatgagtggaccccacagaatggctatgtcacctctgcgcagaagctacagagaagaaagatcttgtctgcagtgcagtcagaagttgacgcactatatgccgcgaactcttaa

SEQ ID NO: 5 - protein, 733 aa, Eremothecium gossypii
MTKASVVDQSAPAYAPKRLLAEARAASKVNIEQVFAFLEGSPEKAALTNELLAEFAADPAITQGPEYYDLTKAEQREQTVKKIARLALYLENDIKLARKQHHKDVVRDLQSPDAPMVTMSDMERFEKRSTLVALIDPQLATRLGVNLSLFGNAVRGNGTDEQIKYWLQERGLIFVKGIYGCFAMTELGHGSNVANLQTRATYDPASDSFVIQTPDLVATKWWIGGAAHSATHSTVYARLIVEGKDYGVKVFVVPLRNPKTMELLAGISIGDIGSKMGRDGIDNGWIQFNNVRIPREYMLSRFTKVIPGNPPKVEMEPLLDSISGYAALLSGRVSMVLDSYRFGARFSTIATRYAFGRQQFGDPTNETQLIEYPLHQFRVLPQLAIIYMMAPGAMKLMDTYNSCLGELYGAGDDKKKLTTVSARMKDLFVESASLKATCTWLTSTLIDELRQTCGGHGYSSYNGFGKAYNDWVVQCTWEGDNNVLCLTSGKSLLKKFAGIVRGKKVTICDTSMDYLRMDYIQKVVMGGTKKVSNLSTLPDYYQIWSVILVKYLKRCAETVRDNNDPESVSKLLVSIAKFHAFYSMLQEFHRKLASDQSHVGDAATKEVLWKVYKLSSLYFIDKFSGEFQQLKVMSPDQMTNVQEQMLAILPEIKTHAIRLTDAFHLPDAVINSSIGNYDGDIYHNYFNDVTRVAAKDKAPGVPPYADMLVNFLARGDQFDNLNISETSFKNLGK

SEQ ID NO: 6 - DNA, 2202 bp, Eremothecium gossypii
atgacaaaggcatcagtggtggaccagtccgcgccggcgtacgcgcccaagcggctgctggcagaggcgcgcgcggcgtcgaaggtgaacatcgagcaggtcttcgcgtttctggaaggctcgccggagaaggcggcgctgacgaacgagctactggcggagtttgcagccgaccctgcgatcacgcagggcccggagtactacgacctcacaaaggccgagcagcgggagcagacggtgaagaagatcgcgcggctggcgctgtacttggagaatgacattaagctggcacgcaagcagcaccacaaggacgtggtgcgggacctgcagtcgccggacgcgccgatggtgactatgagcgacatggaacgcttcgagaagcgctcaacgctggtggcgctgatcgacccgcagctggcaacgcggctgggcgtgaacctgagcttgttcggtaatgccgtgcggggtaacggcacggacgaacagatcaagtattggctgcaggagcgcgggctcatcttcgtgaagggcatctatggctgcttcgcgatgacagagctaggccatgggtccaacgtggcgaacctgcagacacgcgctacgtacgaccctgcgagcgactcgtttgtgattcagacgcccgaccttgtcgcgacgaagtggtggatcggcggtgctgcgcacagcgcgacgcactcgaccgtgtacgcccgtctgatcgtggagggcaaggactacggcgtgaaggtcttcgtggtgcctctgcgcaaccccaagaccatggagttgctggccgggatttccatcggcgacatcggctccaagatgggccgcgacggtatcgacaacggctggatccagtttaacaatgtgcgtattccccgtgagtacatgctgagccggtttacgaaggtgatccccggcaacccgccaaaggttgagatggagcctctgttggactccatctccggctacgccgcgttgctgtccggacgtgtgagcatggtattggactcctaccgctttggcgcacgcttctccaccatcgccacgcggtatgcctttggcagacagcagtttggtgacccaaccaatgagacccagctaatagagtacccattgcaccagttccgtgttctccctcagcttgccataatatacatgatggcgccgggcgcgatgaagttgatggacacatacaacagctgtttgggtgagttgtacggtgctggcgatgacaagaagaagttgactactgttagcgccagaatgaaggacttgtttgtggagtctgccagtttgaaggccacctgcacttggttgacttcgacgttgatcgacgagttgagacagacctgcggtggccacgggtactccagctacaacggtttcggaaaggcatacaacgactgggtcgttcagtgcacttgggaaggtgacaacaacgttctgtgtttgacctctggtaagtcgctgctcaagaagttcgctggtattgttcgtggcaagaaggtgactatctgtgacacctccatggactacctccgcatggactacatccagaaggtggttatgggcggcaccaaaaaggtgagcaacttatccacacttccagactactaccagatctggtcggttatcttggtgaagtacttgaagcgctgcgccgagactgtccgtgacaacaacgacccagaatctgtgtccaagctgctcgtgagtatcgccaagttccacgcattctactctatgctccaggagttccaccgcaagttggcctctgaccagagccacgtgggcgacgccgcaaccaaggaggtcttgtggaaggtctacaagctctcctcgctctacttcatcgacaagttcagcggcgagttccagcagttgaaggtcatgtccccagaccagatgacgaacgtgcaggagcagatgttggctatcctgcctgagatcaagacacacgccatccgtctaactgacgccttccacctccctgacgccgtgatcaactcatctatcggcaactacgacggcgacatctaccacaactacttcaacgatgtcacccgtgttgccgccaaggacaaggctccaggtgtgcccccatacgcggacatgcttgtcaacttcttggctcgtggcgaccagttcgacaatttgaacatcagcgagacctccttcaagaaccttggcaagtag

SEQ ID NO: 7 - protein, 891 aa, Eremothecium gossypii
MSLTFNDRVVIITGAGGGLGREYALDYAKRGAKVVVNDLGGTLGGSGHDTRAADKVVEEIRKAGGTAVANYDTVTDGDKIVKTAIDAFGRVDVIVNNAGILRDGSFAKMTEKNFSAVVDVHLNGSYKLCKAAWPYMRQQKYGRIVNTASPAGLYGNFGQTNYSAAKLGLVGLSETLAKEGHKYNIKVNVIAPIARSRMTEGLLPDHVIRVMGPEKVVPMVVYLTHENTEVTNSIFEPGAGYYTQVRWERSSGGLFNPDEKTFTPEAILHKFPEVLDFKDKPFKAVEHPYQLADYNDLISKARQLPPNEQGSVQVKSLKDKVVIITGAGAGLGRSHALWFAKYGARVVVNDLKGADGVVAEINSQYGEGRAVADSHNIVTDAAAVVETAMKAFERVDVLVNNAGILRDRSFVKMTDDEWNSVLQVHLLSVFALSKAVWPIFMQQRSGVIVNTTSTSGIYGNFGQANYSAAKAAVLGFSKSLAIEGAKRGIRVYVIAPHAFTNMTKTIFGETEIKSSFEPSQVSPFVVLLASNEFARKYRRSVGSLFEVGGGWIGHTRWQRAKGAVSLELATAEFIRDNWATITDFSKPSYPASIDAAGNDMMKAIMTATALQSSTGALKYTSRDSIIYNLGLGANTTELKYVYENHPAFQVLSTYPIVLAMNAGFVDFPSFADNFDYNMLLHGEQYMKLNQYPVPTEGSVKVETAPVASTNKGKKAALIVIGYKVIDAKTNKQLAYTEGSYFVRGAQVPESKKVLTERPTFSTTSFSSPDREPDFEAEIDTSVHQAALYRLAGDYNPLHIDPKVSSIARFPKPILHGLCSLGCTAKALFEKFGQYDELKTRFSSFVFPGDKLKVRAWKEDGGIVIFETIDLDRDMPVLTNSAIKLVGSQSKL

SEQ ID NO: 8 - DNA, 2676 bp, Eremothecium gossypii
atgtcgctaactttcaacgaccgtgtggtaatcattacgggtgccggaggcggtctgggccgtgagtacgcgctggactacgccaagcgcggggccaaggtggtggtgaacgacctaggggggacgcttggcgggtccgggcatgacacaagggctgcagacaaggttgtggaggaaatccgcaaggccggcggcactgcggtggccaactacgacacggtgacggacggtgataagatcgtgaagactgcgatcgacgcgttcgggcgtgtggacgtgattgtcaacaacgcgggcatcttgcgcgacgggtcctttgccaagatgaccgagaagaacttcagcgcggtcgtggacgtgcacctaaacgggtcatacaagctctgcaaagcggcatggccttatatgaggcagcagaagtacgggcgcattgtcaacacggcgtcgcccgccggcttgtacggtaactttggccagacaaactactccgcggccaagctgggtctagttgggctatctgagacgctcgcgaaggaggggcacaagtacaacatcaaggtcaacgtcattgcgcctattgccaggtcgagaatgactgagggtttgcttcctgatcacgtgatcagggttatgggccctgagaaggtggttcccatggttgtgtacttgactcacgagaacaccgaggtcaccaacagcatatttgagccaggcgctggatattacacgcaggtgaggtgggagcgtagctccggcggacttttcaaccctgatgagaagacgtttactcctgaggccattcttcacaagttccctgaggtcctggatttcaaggacaagcccttcaaagctgttgaacacccttaccaactagcagactacaacgatttgatttccaaggcgcggcagttgccacctaacgagcaaggcagcgtgcaggtgaagtccttgaaggacaaggttgtaattattaccggtgctggtgccgggttgggcaggtctcatgctctttggtttgcgaagtacggcgcccgcgtggttgtgaacgacctaaagggtgctgacggcgtggttgctgagatcaacagccagtacggtgaaggccgtgcggtcgctgacagccacaacatcgtgaccgacgccgcggccgtcgtggagactgcaatgaaggctttcgagcgtgttgatgtattggttaacaatgccggtattttgcgtgaccgctcgtttgtgaagatgactgacgatgagtggaatagcgtcctgcaggtgcatttgttgtctgtgtttgcactaagcaaggctgtatggcctatcttcatgcaacagcgctctggtgttattgttaataccacttctacctctggtatctacggtaactttggccaggccaactactctgccgccaaggctgctgttttggggttcagtaagtctttagccattgagggtgccaagcgtggtatcagagtttacgtgattgctcctcacgccttcactaacatgaccaagaccatcttcggcgagaccgagatcaagagctcttttgaacctagtcaggtttctccatttgtcgtcttgcttgcctcgaacgaatttgcaagaaagtacagacggagtgtcggttcgctgtttgaagtcggtggtggctggatcggccacaccagatggcagagagccaagggtgctgtcagtttggagttggctactgccgagttcattagagacaactgggccactatcaccgacttctctaaaccttcatacccagccagtattgatgcggccggtaatgatatgatgaaggcgatcatgactgctaccgctcttcagagcagcactggtgctctaaagtacacttctcgcgacagtatcatttacaaccttggtcttggcgctaacaccacggagttgaagtatgtctatgagaaccacccagccttccaagttctctcaacttacccaattgttctagctatgaacgcgggcttcgttgacttcccttcatttgcggacaacttcgactacaatatgttgcttcacggtgaacagtatatgaagctgaaccagtatccagttccaactgagggtagcgtgaaggtcgagacagcacccgttgcgtctacgaacaagggcaagaaggctgctttgatcgttatcggttataaggttattgacgccaaaacgaacaagcaacttgcctacactgagggctcttatttcgttagaggcgcacaagtccctgagagcaagaaggttttgactgaacgtccaacgttctctacgacttctttctcctcccctgacagggagccagacttcgaagctgagattgacaccagtgttcaccaggccgctttgtacagattggccggtgactacaaccctctacacatcgatccaaaggtttccagtattgcccgcttcccaaaacctatcttgcatgggttgtgttccctgggatgcactgccaaggccctatttgagaaattcggccagtatgatgagttgaagaccagattctccagcttcgtcttccctggtgataagctaaaggttagagcctggaaggaagatggtggcatcgttatctttgagactatcgatctcgacagagatatgcctgtgttgaccaacagtgctatcaagcttgtgggcagccagtccaagctatga

SEQ ID NO: 9 - protein, 403 aa, Eremothecium gossypii
MSSRLNNIKDHVTGQSQATVKGTSPDDVVIVAAYRTAIAKAFKGGFHEMPSDQLLYEFLVKFFEKVDVDKKLIQEVTCGNVLNLGAGANEHRAACLAAGVPFNVPFMAINRQCSSGLTAVNDIANKIKVGQINVGLALGVESMSVNYPRMNFDHTSPDLQENKEARKCYIPMGITNENVAKAFKIPRAVQDEFAADSYKKAEAAVKGGLFQEEILPITNPDGKVINTDEGPRKGVTAESLGKLRPAFIPEKGVTTAGNASQVSDGAAGVLLARRSVAEKLGLPILGKYVAFQAVGVPPEIMGVGPAYAIPAVLEQTGLQVGDVDIFEINEAFAGQALYCVEKLGIDKTKLNPRGGAIALGHPLGCTGARQIATIMRELQPGQIGLTSMCIGSGMGAAAIFVKE

SEQ ID NO: 10 - DNA, 1212 bp, Eremothecium gossypii
atgtcgagcagattgaacaacatcaaggaccacgtcacaggccagtcgcaggccaccgtcaagggcacaagccctgacgacgtggtgatcgtggcagcataccgtactgccatcgccaaggcattcaagggggggttccacgagatgcccagcgaccagctgctctacgagttcttggtcaagttcttcgagaaggtggatgtggacaagaagctgatccaggaggtcacatgcggtaacgtgttgaacctgggcgcgggcgctaacgagcaccgcgctgcctgcctggccgcgggcgtgcccttcaacgtgccattcatggcgattaatagacagtgttcctcggggttgactgcggtaaacgacattgccaacaagatcaaggtcgggcagatcaatgtcgggcttgcgcttggcgtggagtccatgtcggtcaactacccacgcatgaacttcgaccacacctcgccagacctacaggagaacaaggaggcgcgcaagtgctacattcctatgggaatcacgaacgagaacgttgcgaaggccttcaagatcccccgcgctgtccaggacgagtttgctgcggattcttacaagaaggctgaggcggcggtcaagggcggtctgttccaggaggagattttgccaatcaccaatccagatgggaaggtgatcaacaccgacgagggcccaagaaagggcgtgaccgccgagagcctcggcaagttgcgtcctgccttcatcccagagaagggtgtcaccactgctggtaacgcatcccaggtttcggacggtgccgcgggtgttctgctagccagaagatctgttgccgagaaattgggtctgcctatcctaggcaaatatgtcgcattccaggctgtcggtgtgcctccagagatcatgggtgttggtcctgcctacgcaattcctgccgtgttggagcagaccggcttgcaggtcggcgacgtcgacatcttcgagatcaatgaggcttttgcaggccaggccttgtactgtgttgagaagttgggtatcgacaagacgaagctaaacccacgcggtggtgccattgcccttggccacccacttggttgcactggtgcgcgccagattgctactattatgcgggaactacagcctggtcagattggtctaaccagtatgtgtatcggtagtggtatgggtgccgctgccatttttgttaaggaatga

SEQ ID NO: 11 - protein, 462 aa, Eremothecium gossypii
MPAVLDRPKTYKKPTLEDVDPTINYIPAVVRDKLTHESQEMLQRLRKFVDIECLGKEKLYLRELQAHGYESEQCPTVQLLRTRAEELDLQQLYVRKRVFDNQEPFYENRLSMLEYCMASFFLGKSQLAQAVMHAHCSMVNVGAMELLLRHGSPEQLSLFLSPMISAQLHSSFMVSESEVSSSDALNVSTTCKIDDSNGTMTLNGSKWFVTSLEDNKCELWLLLAVTEFDEGNIYKRHSVVLLDKDAIKSEGITYERIDTGGPNSITDSNKYYRVQFKNAVVPLNILGQRGEGFSMVQTRTSLAKLYQCMKLCGTGHEALRLAQLRASSRKVFGSKLQKTDTFKTDVATWKIKIEVCKLLCCNAAVRCQVEGIKVARDDIAMAKIYTPREMSELVNWSIQIHGALGLCTLESPLLHMWHSCRATRINEGPDEALLSQLGKLEISNFAKNQKTWDDELAKAKSS

SEQ ID NO: 12 - DNA, 1389 bp, Eremothecium gossypii
atgccagcagtgttagatagaccgaagacttataagaagccaaccttggaggacgttgatcccactattaattacattcctgcagtagtacgcgataagcttacgcatgagtcacaggagatgctccagcgactccgcaagttcgtggatattgaatgtttggggaaagagaaactgtatctgcgtgaattgcaagcgcatgggtatgaaagcgagcaatgccccacggtgcagcttcttcgcactcgcgccgaagagctcgaccttcaacagctctacgtcaggaagagagtcttcgacaaccaggagccgttctatgaaaaccgcttgagtatgctggaatattgcatggcgagctttttcctcggtaagtcacagttggcacaagcggtcatgcatgcacactgttcgatggtcaatgttggagccatggagctacttttgcgtcatggctccccagaacagttgagcctttttttgagtccaatgatatctgcgcagttacattcgagtttcatggttagcgaaagcgaagtttccagctccgacgcgctcaatgtcagcacaacctgcaaaatagacgattcaaacggcacaatgactttaaatggatcaaagtggtttgtaacatctttggaggacaataagtgtgaactatggcttctacttgctgtaaccgaatttgatgaagggaatatatacaaacggcattcagtggtactcttagacaaggacgctataaaatctgaaggaataacctatgaacgtattgatacaggcgggcccaactccatcacagacagcaataaatactaccgtgtgcaattcaaaaatgcagtagtcccattaaacatcctaggccagagaggtgaaggcttttcgatggtgcaaaccaggactagtttagcaaaactataccaatgcatgaagctctgcggaaccggtcatgaagccctccgcttggcgcaattacgtgcctctagccgaaaggttttcggctcgaagttacaaaagacagacacattcaaaactgacgttgccacttggaaaatcaaaattgaagtttgcaagctgctttgttgcaacgcagctgttcgttgtcaggtagagggtattaaggtggcacgcgatgacatagcgatggccaagatatatacaccacgcgaaatgagcgaattagtaaactggtctattcaaatacacggcgccctgggactctgtaccttggaatccccgctactccatatgtggcatagctgcagggcaacacgcattaacgagggcccagacgaagctcttctttcccaactgggtaagctcgaaatctctaacttcgctaagaatcaaaaaacctgggacgacgagttggccaaggccaaatcctcttaa

SEQ ID NO: 13 - protein, 397 aa, Eremothecium gossypii
MSENVYIVAAARTPIGSFQGSLASQTYVDLGAHAVKAALSQVPQIDASQVDEIIFGNVLSANVGQAPARQVALAAGLPKSIVATTVNKVCASGMKALILAAQAIKCGTADIVVAGGAESMTNTPYYMPAARGGARFGEAKLIDGIQRDGLNDAYDHQAMGVHAEKCASDHSITREEQDNFAIESYQKAQKAHAEGKFAAEIAPVTIKGVRGKPDVTVSQDEETTKFNAEKLKAARPVFKKENGTVTAPNASPINDGGAAIILVSERKLKELNLRPSALIKGWGEAAHEPADFTWAPSLAIPKALKHAGIQDINEVDFVELNEAFSVVGLANTKLLGLDPSKVNVYGGAVALGHPLGCSGARIIVTLLSILQQEGGKVGVAGICNGGGGASSVVLAKL

SEQ ID NO: 14 - DNA, 1194 bp, Eremothecium gossypii
atgtctgagaacgtttacattgtggcggcagcaagaaccccgattggatcattccaaggatcgctagcctcgcaaacttacgtggacttgggggcacatgccgtgaaggccgcgttatcgcaagtaccccagatcgacgcctcccaggtcgatgagattattttcgggaatgtgctctctgcaaacgtgggccaagctcctgcgagacaggtggcgctcgctgccggcttgcccaagagtattgtagccaccaccgtgaacaaggtttgtgcgtccggaatgaaggcgcttattttggcagcccaggcgatcaagtgtgggaccgcagacatcgtggttgcaggcggtgcggagtccatgaccaacacgccgtactacatgccagcggcgcgtggcggtgcgcgcttcggagaggcgaagctcatcgacggaatccagcgcgatggtctaaatgatgcatacgaccaccaggcgatgggtgtgcatgctgagaagtgcgcatcggaccattcgatcacgcgtgaggagcaggacaactttgcgatcgaatcgtaccagaaggcccaaaaggcccatgcagagggcaagtttgcagccgagatcgcgccagtgacgatcaagggtgttagggggaagccagatgtcaccgtgtcgcaggatgaagagaccaccaaattcaatgctgagaaattgaaggctgcgagacccgttttcaagaaggaaaacgggactgtcacagcaccaaacgcttctccaatcaacgatggtggtgcggccatcatccttgtatccgagcgcaagctaaaggagttgaacctccgcccatccgctttgattaagggttggggtgaggctgcccacgagccagcagacttcacctgggcgccctccctcgccattccaaaagcattgaagcatgcagggatccaggacattaacgaggtcgacttcgttgagctaaacgaggccttctccgtggtcggcttggcaaacaccaagcttttgggcctggacccttcaaaggtcaacgtttacggtggtgcagtggccttgggccaccccctaggctgttctggtgcccgtatcattgtcactctactctccattctacagcaggagggtggtaaggtaggtgtagctggtatctgtaacggcggcggcggagcgtcgtccgtggtgctagcaaagttgtag

SEQ ID NO: 15 - protein, 569 aa, Eremothecium gossypii
MSADCSVGANPLAQLNKRVQQDRTLQHGSHVNIHQGAEAQAFKSGPQVSESNKFQMEQFMAGKASSGGNMFMGAGMSSGPLALGGSSGLRMSPGPAKELGARLGGAPMTGSWSQEFNQQVGSPVQSSSAVSSVSMSSASSSVARAGAYRPMNMMRPVMGLQGARAVGVERHAGPAINDAAWEQQFQELEKQVEKTLNISDPVEQQQVLEELSAEAREADYAGGDYEKRFQQIWNDIHDQTDDLDSRTELGGGSGDYQRVFSTRPAQTAQYAFETDNQYLHNTDAYKIGCILMENGAKLSEAALAFEAAVQQDPGHVDAWLRLGLVQTQNEKELSGINALEQCLKADPHNLMALMTVAISYINEGYDVSAFTMLGRWLETKYPAFVEEPLDRVDRYNLSRLIIEQYLRVANALPEVDPDVQLGLGILFYANEDFDKTIDCFRAALAVRPDDECMWNRLGASLANSNRSEEAIQAYHRAIQLKPTFVRARYNLAVSSMNIGCYREAAEHLLTALSMHEVEGVAMAPGSGNVPSSNILETLKRAFIAMDRRDLLERVVPNMDLQQFRGEFNF

SEQ ID NO: 16 - DNA, 1710 bp, Eremothecium gossypii
atgagtgcagactgctctgtgggcgctaacccgctggcgcaactgaacaagcgggtgcagcaggacaggacactgcagcatggatcgcacgtgaatatacaccaaggcgcggaggcgcaggcgttcaagagtggaccgcaggtgagcgagtcgaacaagttccagatggagcagttcatggccgggaaggcgagcagcggcgggaacatgttcatgggggcggggatgagctcggggccactggcgctgggcgggagctcggggctgcggatgtcccccgggccggcgaaggagctcggggcgcgcctggggggcgcgccgatgacggggagctggtcgcaggagttcaaccagcaagtggggagtccggtgcagtcgagctcggccgtgtcgtcggtgtcgatgagctccgcgtcgtcgtcggtggcgcgcgcgggcgcgtacaggcccatgaacatgatgcggccggtgatggggctgcagggcgcgcgggcggtgggcgtggagcgccacgcagggccggcgatcaacgacgcggcgtgggaacagcagtttcaggaactggagaagcaggtggagaagacgctgaacatctcggacccggtggagcagcagcaggtgctggaggagctgagcgcggaggcgcgcgaggcggactacgcaggcggcgactacgagaagcgcttccagcagatatggaacgacatacacgatcagacggacgacctggatagccgaacggagctgggcggcggctcgggcgactaccagcgcgtgttctccacgcggccggcgcagaccgcgcagtacgccttcgagaccgacaaccaatacctgcataacacggacgcgtacaagatagggtgcatcctgatggagaacggcgcgaagctgagcgaggccgcgctcgcgttcgaggccgcagtgcagcaagacccgggccacgtggatgcgtggttgcgcctggggctcgtgcagacgcagaacgagaaggagctcagcggcatcaacgcgctcgagcagtgcctcaaggccgacccgcacaacctgatggcgttgatgaccgtcgccatcagctacatcaacgagggctacgacgtcagcgcgttcacaatgctcgggcgctggctggaaactaagtaccccgccttcgtcgaggagcctctcgaccgcgtcgaccgctacaacctcagccgcctgatcatcgagcagtatctgcgcgtggccaacgctctgcccgaggttgaccccgacgtccagctcggcctcggcatcctcttctacgccaacgaggacttcgacaagaccatcgactgcttccgcgccgcgctcgcagtgcgcccggacgacgaatgcatgtggaaccgcctgggcgcgtcgctcgccaactccaaccgctccgaggaggccatccaggcctaccaccgcgccatccagctcaagcccacctttgtccgcgcccgctacaacctcgccgtctcctccatgaacattggctgctaccgcgaggccgcagagcacctgctcaccgcgctctccatgcacgaggtcgagggcgtcgccatggccccgggcagcggcaacgtgccctcctcgaacatcctcgagaccctcaagcgcgccttcatcgctatggatcgccgcgacctcctggagagggtcgtgcccaacatggacctgcagcagttccgcggcgagttcaacttctga

SEQ ID NO: 17 - protein, 828 aa, Eremothecium gossypii
MASGTQRLLQVQKLFIETNRNMFGLDVRRVAARDYLKVLMWHMWTLLQAAKGRRGRRLRALAAAAAAMVVTGSALTVYQFVSGLRRDGRRPGLQRSRSQILLKSGAREIHVPYGDSERTKRVIIKPTHKDRYEHDRFLFKYFDKGSESRIFYSRFLAQLGILWKILIPRLADKNSLWLCLQVFFLVMRTWLSLLIARLDGHIVKDIIAARKKRFMMDIACWFLIAFPASYTNSAIKFLQRKLSLNFRTNLTRYVHDMYLDHRLVFYKLMYDQDASRSVVANVDNSIANDIAKFCDAVTNLFANMAKPVIDLVFFSFYLRDNLGTLGVAGIIMNYFLTGIVLRRYTPPLGKLVSKRSSAEGAYYNYHLNMINNNEEIAFYQGTEVERTKVIKIYENLMEKMLEVDRAKVGYNVIEDYILKYTWSALGYAFASIPIVFAVGKTGQRKEDTNMRDFIVNKRLMLSLADAGSRLMYSIKDISQLTGYTGRVFTLLRVLHRVHSSDFKYGLIEDIPAPVAGQSESTDLSKNVPSDIRGTVQRNFNGIRLENIDVIIPSPKGIQGTKLISKLKFQIPPVVISDMKASSAPVVSSHSVASMLLGPGSSLLILGPNSCGKSSIQRILAEIWPIYNKTGLVSIPAESDLMCIAQRPYFIQGGTFRDQIIYPMSVDRFYEKGHKDRELVRILKEVKLDYLLKRSEGLSYLDFVADWKDILSGGEKQRMNFARIMFHRPKFLVLDEATNAISVDMEDYLFNMLRKCRFNFISISQRPSLIKYHDYLLEITSGTQWQYQTLGSDEAITSIEAEIESLESKLTQLDAWEKEREELKRKLTH

SEQ ID NO: 18 - DNA, 2487 bp, Eremothecium gossypii
atggcgagtggcacgcaacggctgctgcaagtgcagaagctgtttatcgagacaaatcggaatatgttcggattggacgtgcggcgcgtggctgccagggactacctgaaagtgcttatgtggcacatgtggacactgctgcaggctgcgaaagggcggcgtgggcggcgcctgcgcgcgctggcggcagcagcagcagcgatggtagtcacgggctctgccctgacagtgtatcagtttgtatcgggactgcggcgggatgggcgccgaccggggctgcagcggtcgcggtcgcagatcctgctcaagagcggcgcacgagagatccacgtgccgtacggggacagcgagcggacaaagcgcgtgatcatcaagcctacgcacaaggaccgatacgagcacgatcgctttttattcaagtacttcgacaagggcagcgagtcgcggattttttattcacgctttctggcgcagcttggcatcctgtggaagattctgatacccaggctcgcagacaaaaactcgctatggctctgtctacaggtgttctttctggtgatgcggacgtggttgtcgctgctgattgcacggcttgatggccatattgtcaaggatatcatcgcggcgcggaagaagaggttcatgatggacattgcgtgctggtttctgatagcattcccggcttcgtacacgaatagcgcgatcaagttcctgcagcggaagctgagcttgaacttccggaccaatctgacgcgctacgtccatgacatgtacctagaccacaggctggtgttttacaagctgatgtacgaccaagatgccagccgcagtgtggttgcgaacgttgacaactcgattgccaacgatattgccaagttttgcgacgctgtgacaaacctattcgcaaacatggctaagccagttattgatttggttttcttctccttctatctgcgggataacttgggcactttgggcgttgccggcatcatcatgaactatttcctcacaggaattgtgctgaggagatacacccctcctcttggcaagcttgtcagcaagcgctcgagtgccgaaggtgcctactacaactatcatctaaatatgattaacaataacgaggagattgctttctaccaaggaactgaagtcgaacgcacaaaggtgattaagatatatgagaacttaatggaaaaaatgttggaggttgaccgtgccaaggttggatacaatgtcattgaagattacatcttgaagtatacctggtctgcgcttggttatgcgtttgcgtctattccgatcgtatttgcagtgggcaagacgggccaaagaaaggaggataccaacatgcgcgactttattgttaacaagcgtctcatgctgtcattggcagatgcagggagtagattgatgtactcgattaaggatatttcacagctaaccggttatactggtagagttttcactctcctgcgggttcttcaccgtgttcactcttctgactttaagtacggcctgattgaggacattcctgccccagtagcaggacagtcagagtccacggatttgtccaagaacgtgccctcagacatacgtggtacggtacagcgtaacttcaacggtattcgtttggaaaacatcgatgtaatcattccatcccctaagggcatacaaggcaccaagctaattagtaaattaaagttccagatacctcctgtggtgattagtgatatgaaagcgagctctgctcccgtggtttcaagtcacagtgtggcaagcatgcttttgggacctggaagtagtcttttgattttaggacccaacagttgcggcaaatcatctatccagagaattctcgcggaaatttggccaatttacaataagacaggccttgtatctatcccagctgagtcggatctaatgtgtattgctcagcggccttactttatccaaggaggtaccttcagagatcagataatttatcctatgtccgtagaccggttctacgaaaagggacataaggaccgggagctggtgagaatactgaaagaggttaaacttgactacctcttgaagagatcagaagggttgagctatttagactttgtcgcagactggaaagatatcctaagcggtggtgaaaaacagagaatgaactttgcaaggattatgttccataggccgaagttcctcgtcctggatgaagccaccaatgcgatcagtgttgacatggaggattacctcttcaatatgttgcgtaagtgccgcttcaactttatctccatctcacaacgtccatctttgattaagtaccatgattaccttttggaaatcacttctggtacgcagtggcaatatcagacgcttggttctgatgaagctataacgtccattgaggctgaaatcgaatctctagagtcgaaactcacacaacttgatgcctgggagaaggagagagaggaactcaagcggaaacttactcactaa

SEQ ID NO: 19 - protein, 801 aa, Eremothecium gossypii
MLEFYKAHRIRILKASYLVLLVVTVKNLAQGKDAAERKAAGEPQDRKKPRSRANSAINVHYLRSLLGQTNGIESNSDESAAESDDGDYDGPSVDQKRSSFLIKLLLRDPRCLLTFLLQASLLVIRTMLSLRVATLDGILVSKLVKGQFSEFVKVLLGQWMTLGIPASMVNSLLTYTTRLCAVTINRKVSYHLLDKYLSSHHSFYSVNNLPSDKNKALSLAMSESKENSPNTRREANPPIYVSDIPVQFLTRDVGAFSYNASVLLNQLLKPTLDLILCSFKLAQSSSSGMMAEGTLVLGLIVYFSNLCLKLIQPNFVQLTVKRTHLEGYFRSLHSKLRSSNEEIALFKGQSTELWNLDFSFYQLTLFLAGEIKSRALYDFATSFVVKYVWGAAGLILCSIPVFFRSNMAEDVTADFITNRRLLLTASASIGRYVELRRSIQQLKGEALRLTNFNDRLDAGSSSKTDGKKVIIEYDDSKIQFVHVPLVTPANQVLIPELNFELKHGNHLLIIGPNGCGKSSLFRVLGGLWPVLQSFTNPKKPTKLIMPRRNAENGESPIYYLPQRAYMSNLSTFREQIIYPDKINAFEKKYNGDYQRGDKELADILSVLELDDLIAENMALIMAKRSSTEGSGQPTEVSLTEAFGITRNWSEELSVGIQQRLAMARMYYHRPKFAVLDECTSAVSPEMEQKMYTHAQSLNISLISVCHRTTLWHFHNLLLKFDGNGGYTFGPFDPEQRLTDEQRLAELNKIIEQDVPIWKKKLDELVIAKKSNVLRKSQTNLKSLQESRLPLIQGMSPMTSNT

SEQ ID NO: 20 - DNA, 2406 bp, Eremothecium gossypii
atgctggagttttacaaggcacataggatacgcatcctgaaggcatcgtatcttgtgttacttgtcgtgactgtgaagaatcttgctcagggaaaggatgcggcagaaagaaaggcagcaggagagccgcaggatcgcaagaagcccaggtcgcgggcgaacagcgcgataaacgttcactacctgcggtcgctgctcggccagaccaacgggattgagtcgaactcggacgagagcgctgctgagagcgatgacggtgactatgatgggccgtcggttgatcagaagcgttccagtttcctgataaagctgttgctccgggacccgcgatgtttactgacgttcttgctacaggcgtccctgctggtgatcaggacgatgctttcgcttcgtgtagcgacgctggacggtattttggtttcgaagctggtgaaggggcagttcagtgagtttgtaaaggtgctgttggggcagtggatgactcttgggatccctgcgagtatggtgaactctctgttgacatacactactcgcctttgtgccgtgacgattaaccggaaggtcagttatcatttgcttgataagtacctttcctcccatcatagcttctattccgtaaacaacttgcccagtgacaagaacaaagcgctcagcctagccatgtctgaaagtaaggaaaactcgcccaatacccgcagagaggctaacccaccgatctatgtttcggacattcctgtacaatttctaacgagggacgtgggcgcgttctcttacaatgcctctgttcttctcaaccagctgctgaagccaaccttggacttgatcttgtgttcatttaagcttgcacagagttccagcagtggaatgatggccgaagggacgctggtattgggtttgattgtttacttcagtaatttatgtttgaagctcatacagcccaacttcgtacagcttactgtcaagcggacacatttggagggctacttcaggtcattacattccaagttgcgctcaagcaatgaggagatcgctctattcaagggtcaaagtaccgaactatggaatttggatttctctttctaccagctaacattattcttagccggggagattaaatctagagcactgtacgactttgctactagctttgtggtgaagtacgtctggggcgctgctggcttgatattatgctctatccccgtcttctttcgttctaatatggctgaggatgtcaccgctgattttatcaccaacagacgtttactgctaactgcgtccgcatctattggccgctatgtcgaactgcgccgtagtatccagcagctaaagggagaggccctacggttaacaaatttcaatgacaggctagacgctggaagtagttcgaagactgatgggaaaaaggtcattatagaatacgacgattctaagattcaattcgttcatgttccactagtgacacctgctaatcaggtccttatccccgaacttaacttcgagttgaagcatggtaaccatctattgattataggtcctaatggctgcggcaagtcttctcttttcagggtccttggggggttgtggcctgttttgcaatcgtttactaaccctaagaagcctacgaagctaatcatgccgcgtagaaatgctgaaaatggcgaaagtccgatatattatctcccccagagggcatatatgagcaatttatcgactttccgggagcaaatcatttatcctgataagatcaatgcctttgagaagaaatataatggcgactaccagcgtggtgacaaggaacttgctgatatactttctgttctcgaactagacgacctaatcgctgagaatatggctctcataatggcgaagaggagctccacagaaggttctgggcagcctacagaggtttccttaaccgaggcttttgggattacaagaaattggtccgaggagctttcagttggcatccaacaaagattggccatggccagaatgtactatcacagacctaagttcgcggtgttggatgaatgcacatccgcggtgtcacctgaaatggagcaaaagatgtatacccatgcacagagcctcaacatttcattaatctccgtttgccaccgtactacgctgtggcacttccacaacctgctactaaagttcgatggaaatggagggtacacttttggcccatttgacccggagcagcgcctgaccgatgaacagaggttggccgagctcaacaaaattatcgaacaagacgttcccatttggaagaagaaattggatgaattggttattgctaagaagtcgaatgtccttcgcaaatcacagactaatctaaagtctctacaagaatcgcgtctacccttgatacagggcatgtccccaatgactagtaatacttga

SEQ ID NO: 21 - protein, 382 aa, Eremothecium gossypii
MNQDMELPEAYTSASNDFRSDTFTTPTREMIEAALTATIGDAVYQEDIDTLKLEQHVAKLAGMEAGMFCVSGTLSNQIALRTHLTQPPYSILCDYRAHVYTHEAAGLAILSQAMVTPVIPSNGNYLTLEDIKKHYIPDDGDIHGAPTKVISLENTLHGIIHPLEELVRIKAWCMENDLRLHCDGARIWNASAESGVPLKQYGELFDSISICLSKSMGAPMGSILVGSHKFIKKANHFRKQQGGGVRQSGMMCKMAMVAIQGDWKGKMRRSHRMAHELARFCAEHGIPLESPADTNFVFLDLQKSKMNPDVLVKKSLKYGCKLMGGRVSFHYQISEESLEKIKQAILEAFEYSKKNPYDENGPTKIYRSESADAVGEIKTYKY

SEQ ID NO: 22 - DNA, 1149 bp, Eremothecium gossypii
atgaatcaggatatggaactaccagaggcgtacacgtcggcttcgaacgacttccgttcggacacgttcaccactccaacgcgcgaaatgatcgaggctgcgctaacggcgaccatcggtgacgccgtctaccaagaggacatcgacacgttgaagctagaacagcacgtcgccaagctggccggcatggaggccggtatgttctgcgtatctggtactttgtccaaccagattgctttgcggacccacctaactcagccaccatattcgattctttgcgactaccgtgcgcatgtgtacacgcacgaggctgcggggttggcaattttgtcccaggccatggtgacacctgtcattccttccaacggcaactacttgactttggaagacatcaagaagcactacattcctgatgatggcgacatccacggtgctccaacaaaggttatctcgttggaaaacaccttgcacggtatcattcacccactagaggagcttgttcggatcaaggcttggtgtatggagaacgacctcagactacactgcgatggtgcgagaatctggaacgcgtccgcagaatccggtgtgcctctaaaacagtacggagagctattcgactccatttccatctgcttgtccaagtccatgggtgccccaatgggctccattctcgtcgggtcgcacaagttcataaagaaggcgaaccacttcagaaagcagcaaggtggtggtgtcagacagtctggtatgatgtgcaagatggcgatggtggctatccagggtgactggaagggcaagatgaggcgttcgcacagaatggctcacgagctggccagattttgcgcagagcacggcatcccattggagtcgcctgctgacaccaactttgtctttttggacttgcagaagagcaagatgaaccctgacgtgctcgtcaagaagagtttgaagtacggctgcaagctaatgggcgggcgtgtctccttccactaccagatatctgaggagtcccttgagaagatcaagcaggccatcctagaggcgttcgagtactcgaagaagaacccttacgatgaaaacggccccacgaagatctacagaagtgagtccgctgacgctgtgggtgagatcaagacctacaagtattaa

SEQ ID NO: 23 - protein, 469 aa, Eremothecium gossypii
MPYHLSESHKKLISSHLSESDPEVDAIIKDEIDRQKHSIVLIASENLTSTAVFDALGTPMCNKYSEGYPGARYYGGNQHIDRMELLCQRRALEAFHVTPDRWGVNVQSLSGSPANLQVYQALMKPHERLMGLHLPDGGHLSHGYQTETRKISAVSTYFESFPYRVDPETGIIDYDTLEKNAVLYRPKILVAGTSAYCRLIDYKRMREIADKVGAYLMVDMAHISGLVAAGVIPSPFEYADIVTTTTHKSLRGPRGAMIFFRRGVRSVHPKTGEEVMYDLEGPINFSVFPGHQGGPHNHTISALATALKQATTPEFREYQELVLKNAKVLETEFKKLNYRLVSDGTDSHMVLVSLREKGVDGARVEHVCEKINIALNKNSIPGDKSALVPGGVRIGAPAMTTRGMGEEDFARIVGYINRAVEIARSIQQSLPKEANRLKDFKAKVEDGTDEIAQLAQEIYSWTEEYPLPV

SEQ ID NO: 24 - DNA, 1410 bp, Eremothecium gossypii
atgccataccacctatccgaatcgcacaagaagctcatctcctcgcacctgagcgagagcgacccagaggtggacgcgatcatcaaggatgaaattgacaggcaaaagcactcgattgtgctgattgcgtcggagaacttgacgtcgaccgccgtgttcgacgcgctaggaacgccgatgtgcaacaagtactcggagggctaccccggcgcgcgctactacggcgggaaccagcacatcgaccgcatggagctgctgtgccagcgccgcgcgctggaggcgttccacgtgacgccggaccgctggggcgtcaacgtgcagtcgctgtccgggtcgcccgcgaacctgcaggtgtaccaggcgctgatgaagccgcacgagcggctgatgggtctgcacctgcccgacggcgggcacctgtcgcacggctaccagacggagacgcgcaagatctccgcggtatcgacgtacttcgagtcgttcccgtaccgcgtggacccggagaccggcatcatcgactatgacacgctcgagaagaacgcggtgctgtaccggcccaagatccttgtggcgggcacctccgcgtactgccggctgatcgactacaagcggatgcgcgagatcgccgacaaggtcggtgcgtacctgatggtcgacatggcgcacatctcggggctggtcgccgcaggcgtcatcccctcgcccttcgagtacgccgacatcgtcaccaccaccacccacaagtcgctcagaggcccacggggagccatgatcttcttcaggagaggcgtgcgctccgtgcacccaaagaccggcgaggaggtaatgtacgacctcgagggccctatcaacttctccgtcttccccggccaccagggcggcccccacaaccacaccatctccgccctggccaccgcgctcaagcaggcgacgacccccgagttcagggagtaccaggagctcgtgttgaagaacgccaaggtcctggagaccgagttcaaaaagctcaactaccgcctcgtctccgacggcaccgactcccacatggtcctcgtctccttgcgcgagaagggcgtcgacggcgcccgcgtcgagcacgtctgcgagaagatcaacatcgccctcaacaagaactccatcccaggcgacaagtccgcgctcgtgcccggcggcgtccgcatcggcgcccccgccatgaccaccaggggcatgggcgaggaggacttcgcccgcatcgtcggctacatcaaccgtgctgtcgagatcgcccgttccatccagcagtcgctgcccaaggaggccaacaggctcaaggacttcaaggccaaggtcgaggacggcaccgacgagatagcccagctcgcccaggagatctacagctggactgaggagtaccctctgcctgtctaa

SEQ ID NO: 25 - protein, 510 aa, Eremothecium gossypii
MCGILGVVLADQSKVVAPELFDGSLFLQHRGQDAAGIATCGPGGRLYQCKGNGMARDVFTQARMSGLVGSMGIAHLRYPTAGSSANSEAQPFYVNSPYGICMSHNGNLVNTMSLRRYLDEDVHRHINTDSDSELLLNIFAAELEKYNKYRVNNDDIFCALEGVYKRCRGGYACVGMLAGYGLFGFRDPNGIRPLLFGERVNDDGTMDYMLASESVVLKAHRFQNIRDILPGQAVIIPKTCGSSPPEFRQVVPIEAYKPDLFEYVYFARADSVLDGISVYHTRLLMGIKLAENIKKQIDLDEIDVVVSVPDTARTCALECANHLNKPYREGFVKNRYVGRTFIMPNQKERVSSVRRKLNPMNSEFKDKRVLIVDDSIVRGTTSKEIVNMAKESGAAKVYFASAAPAIRFNHIYGIDLADTKQLVAYNRTVEEITAELGCDRVIYQSLDDLIDCCKTDIISEFEVGVFTGNYVTGVEDVYLQELERCRALNNSNKGEAKAEVDIGLYNSADY

SEQ ID NO: 26 - DNA, 1533 bp, Eremothecium gossypii
atgtgtggcatattaggcgttgtgctagccgatcagtcgaaggtggtcgcccctgagttgtttgatggctcactgttcttacagcatcgcggtcaagatgctgccgggattgctacgtgcggccccggtgggcgcttgtaccaatgtaagggcaatggtatggcacgggacgtgttcacgcaagctcggatgtcagggttggttggctctatggggattgcacacctgagatatcccactgcaggctccagtgcgaactcagaagcgcagccattctatgtgaatagtccctacggaatttgcatgagtcataatggtaatctggtgaacacgatgtctctacgtagatatcttgatgaagacgttcaccgtcatattaacacggacagcgattctgagctactgcttaatatatttgccgcggagctggaaaagtacaacaaatatcgtgtgaacaacgatgatatattttgtgctctagagggtgtttacaaacgttgtcgcggtggctatgcttgtgttggcatgttggcgggatatggattgtttggtttccgggaccccaatgggatcaggccgctattgtttggtgagcgcgtcaacgatgacggcaccatggactacatgctagcgtccgaaagtgtcgttcttaaggcccaccgcttccaaaacatacgtgatattcttcccggccaagccgtcattatccctaaaacgtgcggctccagtccaccagagttccggcaggtagtgccaattgaggcctacaaaccggacttgtttgagtacgtgtatttcgctcgtgctgacagcgttctggacggtatttccgtttaccatacacgcctgttgatgggtatcaaacttgccgagaacatcaaaaaacagatcgatctggacgaaattgacgttgttgtatctgttcctgacactgcacgtacctgtgcattggagtgtgccaaccatttaaacaaaccttatcgcgaaggatttgtcaagaacagatatgttggaagaacatttatcatgccaaaccaaaaagagcgagtatcttctgtgcgccgcaagttgaacccaatgaactcagaatttaaagacaagcgcgtgctgattgtcgatgattccattgtgcgaggtaccacttccaaagagattgttaacatggcgaaggaatccggtgctgccaaggtctactttgcctctgcagcgccagcaattcgtttcaatcacatctacgggattgacctagcagatactaagcagcttgtcgcctacaacagaactgttgaagaaatcactgcggagctgggctgtgaccgcgtcatctatcaatctttggatgacctcatcgactgttgcaagacagacatcatctcagaatttgaagttggagttttcactggtaactacgttacaggtgttgaggatgtgtacttgcaggaattagaacgttgccgcgctcttaataactcgaataagggtgaagcgaaggccgaggttgatattggtctctacaattctgccgactattag

SEQ ID NO: 27 - protein, 318 aa, Eremothecium gossypii
MSSNSIKLLAGNSHPDLAEKVSVRLGVPLSKIGVYHYSNKETSVTIGESIRDEDVYIIQTGTGEQEINDFLMELLIMIHACRSASARKITAVIPNFPYARQDKKDKSRAPITAKLVAKMLETAGCNHVITMDLHASQIQGFFHIPVDNLYAEPNILHYIQHNVDFQNSMLVAPDAGSAKRTSTLSDKLNLNFALIHKERQKANEVSRMVLVGDVADKSCIIVDDMADTCGTLVKATDTLIENGAKEVIAIVTHGIFSGGAREKLRNSKLARIVSTNTVPVDLNLDIYHQIDISAILAEAIRRLHNGESVSYLFNNAVM

SEQ ID NO: 28 - DNA, 957 bp, Eremothecium gossypii
atgtcgtccaatagcataaagctgctagcaggtaactcgcacccggacctagctgagaaggtctccgttcgcctaggtgtaccactttcgaagattggagtgtatcactactctaacaaagagacgtcagttactatcggcgaaagtatccgtgatgaagatgtctacatcatccagacaggaacgggggagcaggaaatcaacgacttcctcatggaactgctcatcatgatccatgcctgccggtcagcctctgcgcggaagatcacagcggttataccaaacttcccttacgcaagacaagacaaaaaggacaagtcgcgagcaccgataactgccaagctggtggccaagatgctagagaccgcggggtgcaaccacgttatcacgatggatttgcacgcgtctcaaattcagggtttcttccacattccagtggacaacctatatgcagagccgaacatcctgcactacatccaacataatgtggacttccagaatagtatgttggtcgcgccagacgcggggtcggcgaagcgcacgtcgacgctttcggacaagctgaatctcaacttcgcgttgatccacaaagaacggcagaaggcgaacgaggtctcgcggatggtgttggtgggtgatgtcgccgacaagtcctgtattattgtagacgacatggcggacacgtgcggaacgctagtgaaggccactgacacgctgatcgaaaatggggcgaaagaagtgattgccattgtgacacacggtatattttctggcggcgcccgcgagaagttgcgcaacagcaagctggcacggatcgtaagcacaaatacggtgccagtggacctcaatctagatatctaccaccaaattgacattagtgccattttggccgaggcaattagaaggcttcacaacggggaaagtgtgtcgtacctgttcaataacgctgtcatgtag

SEQ ID NO: 29 - protein, 320 aa, Eremothecium gossypii
MATNAIKLLAPDIHRGLAELVAKRLGLRLTDCKLKRDCNGEATFSIGESVRDQDIYIITQVGSGDVNDRVLELLIMINASKTASARRITAVIPNFPYARQDRKDKSRAPITAKLMADMLTTAGCDHVITMDLHASQIQGFFDVPVDNLYAEPSVVKYIKEHIPHDDAIIISPDAGGAKRASLLSDRLNLNFALIHKERAKANEVSRMVLVGDVTDKVCIIVDDMADTCGTLAKAAEVLLEHNARSVIAIVTHGILSGKAIENINNSKLDRVVCTNTVPFEEKMKLCPKLDVIDISAVLAESIRRLHNGESISYLFKNNPL

SEQ ID NO: 30 - DNA, 963 bp, Eremothecium gossypii
atggctactaatgcaatcaagcttcttgcgccagatatccacaggggtctggcagagctggtcgctaaacgcctaggcttacgtctgacagactgcaagcttaagcgggattgtaacggggaggcgacattttcgatcggagaatctgttcgagaccaggatatctacatcatcacgcaggtggggtccggggacgtgaacgaccgagtgctggagctgctcatcatgatcaacgctagcaagacggcgtctgcgcggcgaattacggctgtgattccaaacttcccatacgcgcggcaggaccggaaggataagtcacgggcgccaattaccgcgaagctcatggcggacatgctgactaccgcgggctgcgatcatgtcatcaccatggacttacacgcttcgcaaatccagggcttctttgatgtaccagttgacaacctttacgcagagcctagcgtggtgaagtatatcaaggagcatattccccacgacgatgccatcatcatctcgccggatgctggtggtgccaaacgtgcgtcgcttctatcagatcgcctaaacttgaactttgcgctgattcataaggaacgtgcaaaggcaaacgaagtgtcccgcatggttctggtcggcgatgttaccgataaagtctgcattatcgttgacgatatggcggatacttgtggtacgctggccaaggcggcagaagtgctgctagagcacaacgcgcggtctgtgatagccattgttacccacggtatcctttcaggaaaggccattgagaacatcaacaattcgaagcttgatagggttgtgtgtaccaacaccgtgccattcgaggagaagatgaagttatgcccgaagttagatgtaattgatatctcggcagttcttgcggaatccattcgccgtctacacaatggtgaaagtatctcctacctctttaaaaacaacccactatga

SEQ ID NO: 31 - protein, 552 aa, Eremothecium gossypii
MVAVSLERVQVLGQIDAEPRFKPSTTTVADIVTKEALEFVVLLHRTFNGRRKDLLARRQELQQRLDSGEATLDFLPETRAIREDPTWQGPPLAPGLVNRSTEITGPPLRNMLINALNADVNTYMTDFEDSLAPTWENITYGQVNLYDLIRSRADFAVGSKQYQLRDRFERLATLLVRPRGWHMVDKHVLVDDEPISASILDFGLYFFHNAAKLVEVGKGPYFYLPKMEHHLEAKLWNDIFNVAQDYIGMRRGTVRATVLIETLPASFQMDEIIWQLRQHSAGLNCGRWDYIFSTIKKLRGQSQHVLPDRDQVTMTSPFMDAYVKSLIRTCHRRGVHAMGGMAAQIPIKDDPAANEAALAKVRADKIRELQNGHDGSWVAHPALVPICNEVFRNMGTPNQIHVVPEVHIGARDLVNTSISGGRVTIAGIRQNLDIGLQYMEAWLRGSGCVPINNLMEDAATAEVSRCQLHQWVRHRVKLADTGENVTPDLVRGLLRERTDALARASRAGSSNKFALAAKYLEPEITAERFTDFLTTLLYDEIVTPTNASKSRL

SEQ ID NO: 32 - DNA, 1659 bp, Eremothecium gossypii
atggtagcagtcagcttagaaagagtgcaggtgctcgggcagatcgacgcggagccacggttcaagccgtcgacgacgacagtggcggacatcgtgacgaaggaggcgctggagtttgtggtgctgctgcaccgcacgtttaacgggcggcggaaggacctgcttgcgcggcggcaggagctgcagcagcggttggacagcggggaggcgacgctggacttcctgccggagacgcgggcgatccgcgaggacccgacgtggcaggggccgccgctggcgccgggcttggtgaaccgttcgacggagatcacgggcccgccgctgcggaacatgctgatcaacgcgctgaacgcggacgtgaacacctacatgacggacttcgaggactcgctggcgccgacgtgggagaacatcacgtacgggcaggtcaacctgtacgacttgatccgcagccgcgcggactttgcggtgggcagcaagcagtaccagctgcgcgaccggtttgagcggctggcgacgctgctggtgcggccgcgcgggtggcacatggtagacaagcacgtgcttgtcgacgacgagcccatcagcgcctcgatcctggacttcggactttacttcttccacaacgcggctaagctagtggaggtgggcaagggcccatacttctacctgcccaagatggagcaccacctggaggccaagctgtggaacgacattttcaacgtggcgcaggactacatcggcatgcgccgcggcaccgtgcgtgcgaccgtgctgatcgagactctgcctgcctcgttccagatggacgagatcatctggcagctgcgccaacactccgcaggcctcaattgcgggcgctgggactacatcttcagcaccatcaagaagctgcgcgggcagtcgcagcacgtgctgcctgaccgtgaccaggtcacgatgacctcaccgttcatggacgcctacgtcaagagcctgatccgcacgtgccaccgccgcggcgtacacgccatgggcggtatggccgcgcagatccccattaaggacgaccccgccgcgaacgaggccgcgctcgccaaggtccgcgcagacaagatccgcgagctgcagaacggccatgacggctcctgggtcgcacaccccgcgctcgtgcccatctgcaacgaggtcttccgcaacatgggcacgccgaaccaaatccacgttgtgcccgaggtccacatcggcgcgcgcgacctcgtcaacacctccatctccggcggccgcgtcacgatcgcgggtatccgccagaacctggacatcggcctgcagtacatggaggcctggctgcgcggaagcggctgtgtccccatcaacaatctgatggaggacgccgccaccgcagaggtctcgcgctgccagcttcaccagtgggtccgccaccgcgtcaagctcgcggacactggcgagaacgtcaccccggacctcgtccgcggcctcttgcgcgagcgcaccgacgccctggctcgtgccagtcgcgccggctcatcgaacaagtttgcgctggccgctaagtacctcgagccggagattaccgccgagcgcttcacggacttcctcaccaccttgttgtacgacgagatcgtgacccccacaaacgcctcaaagtcgcgtctctga

SEQ ID NO: 33 - protein, 743 aa, Eremothecium gossypii
MAASVPKSNAAEDIKSKKMKCRRQKINPLDVTESLGYQTHRRGIRKPWSKEDDDVLRNAVHQSLLELGYPEGIESIRTIRESQEVCKQIPWEKVVLYFDTKVRKPKDVRKRWTSSLDPNLKKGRWTPEEDRLLLESYQRHGPQWLKVSQELAGRTEDQCAKRYIEVLDPSTKDRLREWTMEEDLALISKVKMYGTKWRQISSEMESRPSLTCRNRWRKIITMVIRGKASETIIQAVESGSEMLSKKGALQDTLRTHSEEQALDDEDGESGTTNSLEHEGPQPGTGAGAHRATGEHPEQNGIPGGRPDGVVETGVPTLLAVNGRPKEPHSAMDMEGSPGYTSESTLFSHQTVRIQSAMSPQGCGLGPNVDNRAKRDAPTPAIQVGGSVNYMEGETNMGFGLPSAHTPVQGPAATPHMQNERMMRSEAINSSLATPEEPPVVNRTVPHLGQCSQQAHPGISGLPDRPAHVLQHAQPRIPDSTFTEWKYSLKGPDGVALGGDILEMSMVEKLVNYSKQNGISISIHQHVHHHYVNTVMPQTHLPDDKRFDAQFGSGFGLTSRQPDAFELDVDLGARTSHYDGLMLDSLPQPPLGNLYMQNYNMSPQPPFSRPPTTSSAGSGKADLSELSPQRKAHFTALPPHVRLQLGSSDASRDSSQRPRKQRRKRLRDPAHSSASSATNTPHSAIASPSSRDHAPSAAPEEEDDFWESLRKLASNPPRTSARAAPRRDSWTAGEPYRGLPYNPS

SEQ ID NO: 34 - DNA, 2232 bp, Eremothecium gossypii
atggccgcatctgttccgaagagcaatgctgcggaggacatcaagagcaagaaaatgaaatgccggcgtcagaaaatcaatcccctggacgttacggaatcgctcggttatcagacacaccggcgtgggatccgcaaaccatggtcgaaggaggacgacgacgtgctgcgtaacgctgtgcaccagagcctgctggaactggggtaccctgaaggcatagagtctatccggaccatccgagaatcacaggaggtctgcaagcaaatcccatgggaaaaggtggtgctgtatttcgacacaaaggtgcgcaagccgaaggatgtgcgcaagcggtggacaagtagtctggaccccaacttgaagaaaggccggtggactcctgaagaggaccggctcttgttggagtcgtaccagcgacatggcccgcagtggctgaaggtgtcgcaggagctagctggtcggactgaggaccagtgcgcgaagcgctatatagaggtgctggacccgagtacgaaagatcggctgcgggagtggacgatggaggaggaccttgcactcatcagcaaggtgaaaatgtacggaacgaaatggcgccagatttcctcagagatggagtcgcggccgagtcttacctgccgtaacaggtggcggaagatcatcacgatggtcatccgcggaaaggcctcagagactattatccaggccgttgaaagcgggagtgaaatgctatcgaagaagggtgctctacaggataccctgcgaacacactcggaagagcaggccttggacgatgaggatggggaaagtggtactacaaattcattggagcatgaaggcccacagccgggcacgggtgccggtgcccacagggcgacaggcgagcatccggaacaaaacggcattcccggcggccgcccggacggagtcgttgagacgggcgtgcccacgctcctggctgtcaatgggcggccgaaagagccacattcagccatggatatggaaggctctcctggctacacgtcggaatcgacgctcttttcgcaccagacagtacgaatccagtccgcgatgtctccccagggctgcggtcttgggccaaacgtggacaatagggcaaaacgcgatgcgccgacccccgctatccaggtaggtgggagtgtcaactatatggagggtgagacaaatatgggcttcggcttgccttccgctcacacgcccgtccaggggcctgctgcaacgccgcacatgcaaaacgaacgcatgatgcggagcgaagcaatcaattcgtcccttgcgacaccagaggaaccccctgtcgtcaatcgcacagttccgcatctggggcagtgctctcagcaggctcaccccgggatttccgggctgcccgaccggcccgcacacgtgttacagcatgcccagccccgcatcccagactcaaccttcacagaatggaagtacagtctcaagggcccggacggggttgcgctcggtggcgacatactggagatgagcatggtcgagaaactggtgaactattccaaacaaaacggcatctcgatctcgattcaccaacatgtgcatcaccattatgtcaacacagtgatgccgcaaacacatctcccagacgacaaacgcttcgacgcgcagtttggcagcggcttcggccttaccagccggcagccggacgcctttgagctggacgtggacctaggcgcgcgcacgtcccactacgacggactcatgttggactcgctgccccagccccccctgggaaatctctatatgcagaactacaacatgtcgccgcaacccccgttctcacgccctccaactaccagctctgccggttctggcaaggcagatctctccgagcttagcccgcaacgcaaggcccattttacagcgctcccgccgcacgtccgcctgcagctcggctccagcgacgccagcagggactcgtcccagcgcccccgaaagcagcgcagaaagcgcctgcgcgaccccgcccactcttctgcctcctctgccaccaacacgccccactccgccattgcctcgccctcctcacgtgaccacgcgccctctgccgcgcctgaggaagaggacgacttctgggagagcctgcgcaagctcgcctcaaacccgccccgtacctccgcccgcgccgccccgcgccgagattcttggactgcgggcgaaccgtaccgtggacttccctataaccccagctag

SEQ ID NO: 35 - protein, 301 aa, Eremothecium gossypii
MTEYTVPEVTCVARARIPTVQGTDVFLHLYHNSIDSKEHLAIVFGENIRSRSLFRYRKDDTQQARMVRGAYVGQLYPGRTEADADRRQGLELRFDETGQLVVERATTWTREPTLVRLHSECYTGETAWSARCDCGEQFDQAGKLMAAATEGEVVGGAGHGVIVYLRQEGRGIGLGEKLKAYNLQDLGADTVQANELLNHPADARDFSLGRAILLDLGIEDIRLLTNNPDKVQQVHCPPALRCIERVPMVPLSWTQPTQGVRSRELDGYLRAKVERMGHMLQRPLVLHTSAAAELPRANTHI

SEQ ID NO: 36 - DNA, 906 bp, Eremothecium gossypii
atgactgaatacacagtgccagaagtgacctgtgtcgcacgcgcgcgcataccgacggtacagggcaccgatgtcttcctccatctataccacaactcgatcgacagcaaggaacacctagcgattgtcttcggcgagaacatacgctcgcggagtctgttccggtaccggaaagacgacacgcagcaggcgcggatggtgcggggcgcctacgtgggccagctgtaccccgggcggaccgaggcagacgcggatcggcgtcagggcctggagctgcggtttgatgagacagggcagctggtggtggagcgggcgacgacgtggaccagggagccgacactggtgcggctgcactcggagtgttacacgggcgagacggcgtggagcgcgcggtgcgactgcggggagcagttcgaccaggcgggtaagctgatggctgcggcgacagagggcgaggtggttggcggtgcggggcacggcgtgatcgtgtacctgcggcaggagggccgcggcatcgggctaggcgagaagctgaaggcgtacaacctgcaggacctgggcgcggacacggtgcaggcgaacgagctgctcaaccaccctgcggacgcgcgcgacttctcgttggggcgcgcaatcctactggacctcggtatcgaggacatccggttgctcacgaataaccccgacaaggtgcagcaggtgcactgtccgccggcgctacgctgcatcgagcgggtgcccatggtgccgctttcatggactcagcccacacagggcgtgcgctcgcgcgagctggacggctacctgcgcgccaaggtcgagcgcatggggcacatgctgcagcggccgctggtgctgcacacgtctgcggcggccgagctcccccgcgccaacacacacatataa

SEQ ID NO: 37 - protein, 584 aa, Eremothecium gossypii
MENTSQDESRKRQVASNLSSDADEGSPAVTRPVKITKRLRKKNLGTGELRDKAGFKLKVQDVSKNRHRQVDPEYEVVVDGPMRKIKPYFFTYKTFCKERWRDRKLLDVFVDEFRDRDRPYYEKVIGSGGVLLNGKSSTLDSVLRNGLISHELHRHEPPVSSRPIRTVYEDDDILVIDKPSGIPAHPTGRYRFNSITKILEKQLGYTVHPCNRLDRLTSGLMFLAKTPKGADEMGDQMKAREVKKEYVARVVGEFPIGEIVVDMPLKTIEPKLALNMVCDPEDEAGKGAKTQLKRISYDGQTSIVKCQPYTGRTHQIRVHLQYLGFPIANDPIYSNPHIWGPSLGKECKADYKEVIQKLNEIGKTKSAESWYHSDSQGEVLKGEQCDECGTELYTDPGPNDLDLWLHAYRYESTELDENGAKKWSYSTAFPEWALEQHGDFMRLAIEQAKKCPPAKTSFSVGAVLVNGTEILATGYSRELEGNTHAEQCALQKYFEQHKTDKVPIGTVIYTTMEPCSLRLSGNKPCVERIICQQGNITAVFVGVLEPDNFVKNNTSRALLEQHGIDYILVPGFQEECTEAALKGH

SEQ ID NO: 38 - DNA, 1758 bp, Eremothecium gossypii
atggaaaacacatcgcaggatgagagtcgcaaaagacaggtcgcttcgaacttgagcagcgatgccgatgagggctcgccggcagttacgaggccggttaaaatcaccaaacgcctcaggaagaagaacctcgggacaggcgagctacgggacaaagcaggattcaagttgaaggtgcaagacgtgagcaaaaaccgtcacagacaggtcgatccggaatacgaagtcgtggtagatggcccgatgcgcaagatcaaaccgtatttcttcacatacaagactttctgcaaggagcgctggagagatcggaagttgcttgatgtgtttgtggatgaatttcgggaccgcgataggccttactacgagaaagtcatcggttcgggtggtgtgctcctgaacggtaagtcatcgacgttagatagcgtattgcgtaatggagacctcatttcgcacgagctgcaccgtcatgagccaccggtctcctctaggccgattaggacggtgtacgaagatgatgacatcctggtgattgacaagcccagcgggattccagcccatcccaccgggcgttaccgcttcaactccattacgaaaatacttgaaaaacagcttggatacactgttcatccatgtaaccgactggaccgcctaaccagtggcctaatgttcttggcaaaaactccaaagggagccgatgagatgggtgatcagatgaaggcgcgcgaagtgaagaaagaatatgttgcccgggttgttggggaatttcctataggtgagatagttgtggatatgccactgaagactatagagccgaagcttgccctaaacatggtttgcgacccggaagacgaagcgggcaagggcgctaagacgcagctcaaaagaatcagctacgatggacaaacgagcatagtcaagtgccaaccgtacacgggccggacgcatcagatccgtgttcacttgcaatacctgggcttcccaattgccaacgatccgatttattccaatccgcacatatggggcccaagtctgggcaaggaatgcaaagcagactacaaggaggtcatccaaaaactaaacgaaattggtaagactaaatctgcggaaagttggtaccattctgattcccaaggtgaagttttgaaaggggaacaatgcgatgaatgtggcaccgaactgtacactgacccgggcccgaatgatcttgacttatggttgcatgcatatcggtatgaatccactgaactggatgagaacggtgctaaaaagtggagttactctactgcgtttcctgagtgggctcttgagcagcacggcgacttcatgcggcttgccatcgaacaggctaagaaatgcccacccgcgaagacatcatttagcgttggtgccgtgttagttaatgggaccgagattttggccactggttactcacgggagctggaaggcaacacgcacgctgaacaatgtgcacttcaaaaatattttgaacaacataaaaccgacaaggttcctattggtacagtaatatacacgactatggagccttgttctctccgtctcagtggtaataaaccgtgtgttgagcgtataatctgccagcagggtaatattactgctgtttttgttggcgtacttgagccagacaacttcgtgaagaacaatacaagtcgtgcgctattggaacaacatggtatagactatattcttgtccctgggtttcaagaagaatgtactgaagccgcattgaagggtcattga

SEQ ID NO: 39 - protein, 212 aa, Eremothecium gossypii
MTSPCTDIGTAIEQFKQNKMIIVMDHISRENEADLICAAAHMTAEQMAFMIRYSSGYVCAPMTNAIADKLDLPLMNTLKCKAFSDDRHSTAYTITCDYAHGTTTGISARDRALTCNQLANPESKATDFTKPGHIVPLRARDGGVLERDGHTEAALDLCRLAGVPEVAAICELVSERDVGLMMTLDECIEFSKKHGLALITVDDLKAAVAAKQ

SEQ ID NO: 40 - DNA, 639 bp, Eremothecium gossypii
atgacaagcccatgcactgatatcggtaccgctatagagcagttcaagcaaaataagatgatcatcgtcatggaccacatctcgagagaaaacgaggccgatctaatatgtgcagcagcgcacatgactgccgagcaaatggcatttatgattcggtattcctcgggctacgtttgcgctccaatgaccaatgcgattgccgataagctagacctaccgctcatgaacacattgaaatgcaaggctttctccgatgacagacacagcactgcgtatacaatcacctgtgactatgcgcacgggacgacgacaggtatctccgcacgtgaccgggcgttgacctgtaatcagttggcgaacccggagtccaaggctaccgacttcacgaagccaggccacattgtgccattgcgtgcccgtgacggcggcgtgctcgagcgtgacgggcacaccgaagcggcgctcgacttgtgcagactagcgggtgtgccagaggtcgctgctatttgtgaattagtaagcgaaagggacgtcgggctgatgatgactttggatgagtgtatagaattcagcaagaagcacggtcttgccctcatcaccgtcgatgacctgaaggctgcagttgccgccaagcagtag

SEQ ID NO: 41 - protein, 172 aa, Eremothecium gossypii
MIKGLGEVDQTYDASSVKVGIVHARWNKTVIDALVQGAIEKLLAMGVKEKNITVSTVPGAFELPFGTQRFAELTKASGKHLDVVIPIGVLIKGDSMHFEYISDSVTHALMNLQKKIRLPVIFGLLTCLTEEQALTRAGLGESEGKHNHGEDWGAAAVEMAVKFGPRAEQMKK

SEQ ID NO: 42 - DNA, 519 bp, Eremothecium gossypii
atgattaagggattaggcgaagttgatcaaacctacgatgcgagctctgtcaaggttggcattgtccacgcgagatggaacaagactgtcattgacgctctcgtccaaggtgcaattgagaaactgcttgctatgggagtgaaggagaagaatatcactgtaagcaccgttccaggtgcgtttgaactaccatttggcactcagcggtttgccgagctgaccaaggcaagtggcaagcatttggacgtggtcatcccaattggagtcctgatcaaaggcgactcaatgcactttgaatatatatcagactctgtgactcatgccttaatgaacctacagaagaagattcgtcttcctgtcatttttggtttgctaacgtgtctaacagaggaacaagcgttgacacgtgcaggcctcggtgaatctgaaggcaagcacaaccacggtgaagactggggtgctgctgccgtggagatggctgtaaagtttggcccacgcgccgaacaaatgaagaagtga

SEQ ID NO: 43 - protein, 235 aa, Eremothecium gossypii
MFTGIVEHIGTVAEYLENDASEAGGNGVSVLIKDAAPILADCHIGDSIACNGICLTVTEFTADSFKVGIAPETVYRTEVSSWKAGSKINLERAISDDRRYGGHYVQGHVDSVASIVSREHDGNSINFKFKLRDQEYEKYVVEKGFVAIDGVSLTVSKMDPDGCFYISMIAHTQTAVALPLKPDGALVNIETDVNGKLVEKQVAQYLNAQLEGESSPLQRVLERIIESKLASISNK

SEQ ID NO: 44 - DNA, 708 bp, Eremothecium gossypii
atgtttaccggtatagtggaacacattggcactgttgctgagtacttggagaacgatgccagcgaggcaggcggcaacggtgtgtcagtccttatcaaggatgcggctccgatactggcggattgccacatcggtgactcgattgcatgcaatggtatctgcctgacggtgacggagttcacggccgatagcttcaaggtcgggatcgcaccagaaacagtttatcggacggaagtcagcagctggaaagctggctccaagatcaacctagaaagggccatctcggacgacaggcgctacggcgggcactacgtgcagggccacgtcgactcggtggcctctattgtatccagagagcacgacgggaactctatcaactttaagtttaaactgcgcgatcaagagtacgagaagtacgtagtagaaaagggttttgtggcgatcgacggtgtgtcgctgactgtaagcaagatggatccagatggctgtttctacatctcgatgattgcacacacgcagaccgctgtagcccttccactgaagccggacggtgccctcgtgaacatagaaacggatgttaacggcaagctagtagagaagcaggttgcacagtacctgaatgcgcagctggaaggtgagagctcgccattgcagcgcgtgctcgaaaggattattgaatccaagcttgctagcatctcaaataagtga

SEQ ID NO: 45 - protein, 246 aa, Eremothecium gossypii
MALIPLSQDLADILAPYLPTPPDSSARLPFVTLTYAQSLDARIAKQKGERTVISHEETKTMTHYLRYHHSGILIGSGTALADDPGLNCRWTPAADGADCTEQSSPRPIILDVRGRWRYRGSKIEYLHNLGKGKAPIVVTGGEPEVRELGVSYLQLGVDEGGRLNWGELFERLYSEHHLESVMVEGGAEVLNQLLLRPDIVDSLVITIGSKFLGSLGVAVSPAEEVNLEHVNWWHGTSDSVLCGRLA

SEQ ID NO: 46 - DNA, 741 bp, Eremothecium gossypii
atggcgctaataccactttctcaagatctggctgatatactagcaccgtacttaccgacaccaccggactcatccgcacgcctgccgtttgtcacgctgacgtatgcgcagtccctagatgctcgtatcgcgaagcaaaagggtgaaaggacggttatttcgcatgaggagaccaagacaatgacgcattatctacgctaccatcatagcggcatcctgattggctcgggcacagcccttgcggacgacccgggtctcaattgccggtggacacctgcagcggacggggcggattgcaccgaacagtcttcaccacgacccattatcttggatgttcggggcagatggagataccgcgggtccaaaatagagtatctgcataaccttggcaaggggaaggcgcccatagtggtcacggggggtgagccggaggtccgcgaactaggcgtcagttacctgcagctgggtgtcgacgagggtggccgcttgaattggggcgagttgtttgagcgactctattctgagcaccacctggaaagtgtcatggtcgaaggcggcgcggaggtgctcaaccagctgctgctgcgcccagatattgtggacagtctggtgatcacgataggatccaagttcctgggctcactaggtgttgcggtctcaccagctgaggaggtgaacctagagcatgtgaactggtggcacggaacaagtgacagtgttttgtgcggccggctcgcatag

SEQ ID NO: 47 - protein, 510 aa, Eremothecium gossypii
MCGILGVVLADQSKVVAPELFDGSLFLQHRGQDAAGIATCGPGGRLYQCKGNGMARDVFTQARMSGLVGSMGIAHLRYPTAGSSANSEAQPFYVNSPYGICMSHNGNLVNTMSLRRYLDEDVHRHINTDSDSELLLNIFAAELEKYNKYRVNNDDIFCALEGVYKRCRGGYACVGMLAGYGLFGFRDPNGIRPLLFGERVNDDGTMDYMLASESVVLKAHRFQNIRDILPGQAVIIPKTCGSSPPEFRQVVPIEAYKPDLFEYVYFARADSVLDGISVYHTRLLMGIKLAENIKKQIDLDEIDVVVSVPVTARTCALECANHLNKPYREGFVANRYVGRTFIMPNQKERVSSVRRKLNPMNSEFKDKRVLIVDDSIVRGTTSKEIVNMAKESGAAKVYFASAAPAIRFNHIYGIDLWDTKQLVAYNRTVEEITAELGCDRVIYQSLDDLIDCCKTDIISEFEVGVFTGNYVTGVEDVYLQELERCRALNNSNKGEAKAEVDIGLYNSADY

SEQ ID NO: 48 - DNA, 1533 bp, Eremothecium gossypii
atgtgtggcatattaggcgttgtgctagccgatcagtcgaaggtggtcgcccctgagttgtttgatggctcactgttcttacagcatcgcggtcaagatgctgccgggattgctacgtgcggccccggtgggcgcttgtaccaatgtaagggcaatggtatggcacgggacgtgttcacgcaagctcggatgtcagggttggttggctctatggggattgcacacctgagatatcccactgcaggctccagtgcgaactcagaagcgcagccattctatgtgaatagtccctacggaatttgcatgagtcataatggtaatctggtgaacacgatgtctctacgtagatatcttgatgaagacgttcaccgtcatattaacacggacagcgattctgagctactgcttaatatatttgccgcggagctggaaaagtacaacaaatatcgtgtgaacaacgatgatatattttgtgctctagagggtgtttacaaacgttgtcgcggtggctatgcttgtgttggcatgttggcgggatatggattgtttggtttccgggaccccaatgggatcaggccgctattgtttggtgagcgcgtcaacgatgacggcaccatggactacatgctagcgtccgaaagtgtcgttcttaaggcccaccgcttccaaaacatacgtgatattcttcccggccaagccgtcattatccctaaaacgtgcggctccagtccaccagagttccggcaggtagtgccaattgaggcctacaaaccggacttgtttgagtacgtgtatttcgctcgtgctgacagcgttctggacggtatttccgtttaccatacacgcctgttgatgggtatcaaacttgccgagaacatcaaaaaacagatcgatctggacgaaattgacgttgttgtatctgttcctgtcactgcacgtacctgtgcattggagtgtgccaaccatttaaacaaaccttatcgcgaaggatttgtcgcgaacagatatgttggaagaacatttatcatgccaaaccaaaaagagcgagtatcttctgtgcgccgcaagttgaacccaatgaactcagaatttaaagacaagcgcgtgctgattgtcgatgattccattgtgcgaggtaccacttccaaagagattgttaacatggcgaaggaatccggtgctgccaaggtctactttgcctctgcagcgccagcaattcgtttcaatcacatctacgggattgacctatgggatactaagcagcttgtcgcctacaacagaactgttgaagaaatcactgcggagctgggctgtgaccgcgtcatctatcaatctttggatgacctcatcgactgttgcaagacagacatcatctcagaatttgaagttggagttttcactggtaactacgttacaggtgttgaggatgtgtacttgcaggaattagaacgttgccgcgctcttaataactcgaataagggtgaagcgaaggccgaggttgatattggtctctacaattctgccgactattag

SEQ ID NO: 49 - DNA, 5416 bp, Artificial Sequence
  Plasmid
gaggagtggcatgcaggtgacaagaagtcacgcggccaggctgattacgagcgctacgcgaacagcgactatgcgaagtacgcgcaaagtgaccgttctacactaggtaataagtattagtgtctatgcgctagttgctagctgcttgcattgtgtaatccttcgagaatgatgactatcggaattttgatgcgaacgttttttcgggaacggctgagtaaatagccgttgtaaaaatggcctcgactcattgatctcttgacagaataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtaactgtcgcccgtacatttagcccatacatccccatgtataatcatttgcatccatacattttgatggccgcacggcgcgaagcaaaaattacgggtcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttcccacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggatctgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcccgagaacccttaatataacttcgtataatgtatgctatacgaagttatgtctgggtgcacgacacctgacctccgccccgcgggcttcctgttttcgccgggcgcggcacatggtgcggcttcctccgacaggaagccgggccgccggacgcgcacgtcagaggcgtcaccagggcaaatgggtggaagcgaagggaactacgacgaacggtcagcacccctggggcccccacgctcgcaccacagccgctgcgcgtgggcgtgaaaaattttacctgcgggctctccttacgatctcctattttatttcctggggggcagtcgaaatctatataagagggccccggggcgcacaacgggaggactctggtggagagaccaggaggttgaattaattcagtccacacatacacaccgcacaatggcacagcaactactgaagcaagtgttgcggacccttgcgcttccggtgataatgccgcttttagcgctgaacaggaggtttcggatattggacgatattcggacaatcacgtacttcgttcaggcgttggtagcatacggatggtgcacactgacacaacggttcccaacatggtatgttttcgaggcccaggttgcgaaacatggggattcgccgtgtatccgatactgccgcccgcaggcgcggaagggcgatagtgcatgctcctgcacatcgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgat

SEQ ID NO: 50 - DNA, 5409 bp, Artificial Sequence
  Plasmid
gaggagtggcatgcagtcgccggggtcagctgctgggaacatttccccagcagacccacatcatgccgaaggcaacaacgtgtgttgatcaaagctcgcaccgcgcagccaacatgagggcaggaacgccgcggtatgcgcgggaactacgacagcagccgagagcgatttgccgcgcgggcgtgggatatatatatgtatgtaggttggtagcgaacaccaggacttgagctctctcaccgtgacttagtcgattgtagtcagctataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtaactgtcgcccgtacatttagcccatacatccccatgtataatcatttgcatccatacattttgatggccgcacggcgcgaagcaaaaattacgggtcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttcccacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggatctgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcccgagaacccttaatataacttcgtataatgtatgctatacgaagttatgtctgggtgcacgacacctgacctccgccccgcgggcttcctgttttcgccgggcgcggcacatggtgcggcttcctccgacaggaagccgggccgccggacgcgcacgtcagaggcgtcaccagggcaaatgggtggaagcgaagggaactacgacgaacggtcagcacccctggggcccccacgctcgcaccacagccgctgcgcgtgggcgtgaaaaattttacctgcgggctctccttacgatctcctattttatttcctggggggcagtcgaaatctatataagagggccccggggcgcacaacgggaggactctggtggagagaccaggaggttgaattaattcagtccacacatacacaccgcacaatgacaaaggcatcagtggtggaccagtccgcgccggcgtacgcgcccaagcggctgctggcagaggcgcgcgcggcgtcgaaggtgaacatcgagcaggtcttcgcgtttctggaaggctcgccggagaaggcggcgctgacgaacgagctactggcggagtttgcagccgaccctgcgatcacgcagggcccggagtactacgacctcacaaaggccgagcagcgggagcagacggtgaagaagatcgtagtgcatgctcctgcacatcgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgat

SEQ ID NO: 51 - DNA, 9655 bp, Artificial Sequence
  Plasmid
aaggccggccgcggccgctcgatttaaatgcatggccagtagtcattccgcatctgaagataatcttcgcataatacatgtgcctaaactggttctcttacctatgacctgcaggttggcagtggacttaaccatgcggttttcgtgtggattgtgcatccacgtcatctccgcccgtccacgacaacgctgctgacataaaaaaagtcggccttgtcacatgcctggacaactcataggagcatggatatacgtcctgcggcagaaaggtcaaaaaccaagctagcttatcacctcgaataacttcgtataatgtatgctatacgaagttattaggtagatcgatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttatgctctagagctgtaaaagtctgtcacgtgcccgttgccacacatccgactagggctactcggcattcgcatccgctgccattcatcgcccaacggaactacgcggataaccaccgaagctcgggcctattagtcctgtacatcacggagacatattgtattatataacctgtttccggcaacaaggtgagatgttgggtattggggtagtaacgtccatgattgcacgcagtaattgcgagtccctcggccactgccgatatccgatacaaaaagtatataagtgctatagctgtgctcttaatagccggagcaatctagaacagggtggtgaggctagcgcaaaaagtttcgagcttacacactcagctacaggctacatccagcaggtaaaccaacgcagtatatatgtcgctaactttcaacgaccgtgtggtaatcattacgggtgccggaggcggtctgggccgtgagtacgcgctggactacgccaagcgcggggccaaggtggtggtgaacgacctaggggggacgcttggcgggtccgggcatgacacaagggctgcagacaaggttgtggaggaaatccgcaaggccggcggcactgcggtggccaactacgacacggtgacggacggtgataagatcgtgaagactgcgatcgacgcgttcgggcgtgtggacgtgattgtcaacaacgcgggcatcttgcgcgacgggtcctttgccaagatgaccgagaagaacttcagcgcggtcgtggacgtgcacctaaacgggtcatacaagctctgcaaagcggcatggccttatatgaggcagcagaagtacgggcgcattgtcaacacggcgtcgcccgccggcttgtacggtaactttggccagacaaactactccgcggccaagctgggtctagttgggctatctgagacgctcgcgaaggaggggcacaagtacaacatcaaggtcaacgtcattgcgcctattgccaggtcgagaatgactgagggtttgcttcctgatcacgtgatcagggttatgggccctgagaaggtggttcccatggttgtgtacttgactcacgagaacaccgaggtcaccaacagcatatttgagccaggcgctggatattacacgcaggtgaggtgggagcgtagctccggcggacttttcaaccctgatgagaagacgtttactcctgaggccattcttcacaagttccctgaggtcctggatttcaaggacaagcccttcaaagctgttgaacacccttaccaactagcagactacaacgatttgatttccaaggcgcggcagttgccacctaacgagcaaggcagcgtgcaggtgaagtccttgaaggacaaggttgtaattattaccggtgctggtgccgggttgggcaggtctcatgctctttggtttgcgaagtacggcgcccgcgtggttgtgaacgacctaaagggtgctgacggcgtggttgctgagatcaacagccagtacggtgaaggccgtgcggtcgctgacagccacaacatcgtgaccgacgccgcggccgtcgtggagactgcaatgaaggctttcgagcgtgttgatgtattggttaacaatgccggtattttgcgtgaccgctcgtttgtgaagatgactgacgatgagtggaatagcgtcctgcaggtgcatttgttgtctgtgtttgcactaagcaaggctgtatggcctatcttcatgcaacagcgctctggtgttattgttaataccacttctacctctggtatctacggtaactttggccaggccaactactctgccgccaaggctgctgttttggggttcagtaagtctttagccattgagggtgccaagcgtggtatcagagtttacgtgattgctcctcacgccttcactaacatgaccaagaccatcttcggcgagaccgagatcaagagctcttttgaacctagtcaggtttctccatttgtcgtcttgcttgcctcgaacgaatttgcaagaaagtacagacggagtgtcggttcgctgtttgaagtcggtggtggctggatcggccacaccagatggcagagagccaagggtgctgtcagtttggagttggctactgccgagttcattagagacaactgggccactatcaccgacttctctaaaccttcatacccagccagtattgatgcggccggtaatgatatgatgaaggcgatcatgactgctaccgctcttcagagcagcactggtgctctaaagtacacttctcgcgacagtatcatttacaaccttggtcttggcgctaacaccacggagttgaagtatgtctatgagaaccacccagccttccaagttctctcaacttacccaattgttctagctatgaacgcgggcttcgttgacttcccttcatttgcggacaacttcgactacaatatgttgcttcacggtgaacagtatatgaagctgaaccagtatccagttccaactgagggtagcgtgaaggtcgagacagcacccgttgcgtctacgaacaagggcaagaaggctgctttgatcgttatcggttataaggttattgacgccaaaacgaacaagcaacttgcctacactgagggctcttatttcgttagaggcgcacaagtccctgagagcaagaaggttttgactgaacgtccaacgttctctacgacttctttctcctcccctgacagggagccagacttcgaagctgagattgacaccagtgttcaccaggccgctttgtacagattggccggtgactacaaccctctacacatcgatccaaaggtttccagtattgcccgcttcccaaaacctatcttgcatgggttgtgttccctgggatgcactgccaaggccctatttgagaaattcggccagtatgatgagttgaagaccagattctccagcttcgtcttccctggtgataagctaaaggttagagcctggaaggaagatggtggcatcgttatctttgagactatcgatctcgacagagatatgcctgtgttgaccaacagtgctatcaagcttgtgggcagccagtccaagctatgaggcttctatggtcagttttccagccggtaggattatatgcagttagattaatacgtactagctacgcagtaaatgttttcagtttacattttgtcgatggcgattcctgccattgttaggttctgtccgaggaagccgctctaaataccggtacttgcggccctggcgcacgcgcccgccgcgcatccgctcaaagtagaagtccactacgtgcgcgcccggcattcgctcctggtcaaaagcagacaccggagccgcagcggctccgcgtccagccccgcctacaggtgcgcctgcgccacacaggtacggcaggctgtgcggcgcgcgcgcccaccgctccgcgcctgcccaccgcgcgcccctgcttgcgccgcccgcttcccagcccctttctgcgccgcttcccgcagaccacggccgcatcatatagttgtgcgctagcactaattttacagaagacatctgccgtgctctcgttgctcgtgtcttcattgcgcactcgcgggggaggggcccaggcacaaatctactgctcccgccttttctctgccgccagcttttgagtttgaaatgcaattatccttttgggtcgttaccgtcgctcgatcaggtctgcttcgtgtccggcattttgcggggaacacaaaaatccgtccggagccgcacgtgtccggttgcagtcacgtggggcgaccttccggacagggggataccgggtattatgcctgctcctgggtgtcggacataaggttggggtcccccgcagatgacatggactcgcggtgattgtaacggtaatgaaacaatctccacggcacgagggcatatttgtaaggtatataaggcaaggcgcgggtgggagtacagcggtgcgcgacagttagcaggagataccaagccgaaactatgtcgagcagattgaacaacatcaaggaccacgtcacaggccagtcgcaggccaccgtcaagggcacaagccctgacgacgtggtgatcgtggcagcataccgtactgccatcgccaaggcattcaagggggggttccacgagatgcccagcgaccagctgctctacgagttcttggtcaagttcttcgagaaggtggatgtggacaagaagctgatccaggaggtcacatgcggtaacgtgttgaacctgggcgcgggcgctaacgagcaccgcgctgcctgcctggccgcgggcgtgcccttcaacgtgccattcatggcgattaatagacagtgttcctcggggttgactgcggtaaacgacattgccaacaagatcaaggtcgggcagatcaatgtcgggcttgcgcttggcgtggagtccatgtcggtcaactacccacgcatgaacttcgaccacacctcgccagacctacaggagaacaaggaggcgcgcaagtgctacattcctatgggaatcacgaacgagaacgttgcgaaggccttcaagatcccccgcgctgtccaggacgagtttgctgcggattcttacaagaaggctgaggcggcggtcaagggcggtctgttccaggaggagattttgccaatcaccaatccagatgggaaggtgatcaacaccgacgagggcccaagaaagggcgtgaccgccgagagcctcggcaagttgcgtcctgccttcatcccagagaagggtgtcaccactgctggtaacgcatcccaggtttcggacggtgccgcgggtgttctgctagccagaagatctgttgccgagaaattgggtctgcctatcctaggcaaatatgtcgcattccaggctgtcggtgtgcctccagagatcatgggtgttggtcctgcctacgcaattcctgccgtgttggagcagaccggcttgcaggtcggcgacgtcgacatcttcgagatcaatgaggcttttgcaggccaggccttgtactgtgttgagaagttgggtatcgacaagacgaagctaaacccacgcggtggtgccattgcccttggccacccacttggttgcactggtgcgcgccagattgctactattatgcgggaactacagcctggtcagattggtctaaccagtatgtgtatcggtagtggtatgggtgccgctgccatttttgttaaggaatgaactgtgagcagcgcgcctcgcggcgtccaccgttgcccttggactcgtaaactgttcggcttattttacatagttgcttgctggctcgagttagatttagcatcaatattgaattgcatgggcatgtcatactacatggaaggagaatgagttagaaagtagcacgttggttgcgtaattttgtgacagaaagccgacggcctcgccatttttgcaggcacacggaaggcaggcggcacccagcgtaacaatagccaatggaacatatactatggtggcagcaggcggatccacaatgcagctgcagtacaagcccggtaactttgatggagtagctctcttttacgactatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttta

SEQ ID NO: 52 - DNA, 19 bp, Artificial Sequence
  Primer
cccctctacgatgctctcg

SEQ ID NO: 53 - DNA, 23 bp, Artificial Sequence
  Primer
atagattgtcgcacctgattgcc

SEQ ID NO: 54 - DNA, 24 bp, Artificial Sequence
  Primer
ctgcctcggtgagttttctccttc

SEQ ID NO: 55 - DNA, 22 bp, Artificial Sequence
  Primer
cacgtttgattaggagagaggg

SEQ ID NO: 56 - DNA, 27 bp, Artificial Sequence
  Primer
cacgattctgatactttacttggtctt

SEQ ID NO: 57 - DNA, 18 bp, Artificial Sequence
  Primer
caatgacgcgcgcaaccg

SEQ ID NO: 58 - DNA, 20 bp, Artificial Sequence
  Primer
gctaaatgtacgggcgacag

SEQ ID NO: 59 - DNA, 20 bp, Artificial Sequence
  Primer
gtcgctcatagtcaccatcg

SEQ ID NO: 60 - DNA, 22 bp, Artificial Sequence
  Primer
tcgctatactgctgtcgattcg

SEQ ID NO: 61 - DNA, 17 bp, Artificial Sequence
  Primer
ccagtagtcattccgca

SEQ ID NO: 62 - DNA, 17 bp, Artificial Sequence
  Primer
aacgcatcccaggtttc

SEQ ID NO: 63 - DNA, 19 bp, Artificial Sequence
  Primer
tactccatcaaagttaccg

SEQ ID NO: 64 - DNA, 17 bp, Artificial Sequence
  Primer
ctaccccaatacccaac

SEQ ID NO: 65 - DNA, 20 bp, Artificial Sequence
  Primer
ctgaccgttcgtcgtagttc

SEQ ID NO: 66 - DNA, 23 bp, Artificial Sequence
  Primer
ggtaactggtccggatccgcgcc

SEQ ID NO: 67 - DNA, 22 bp, Artificial Sequence
  Primer
cccgctgctcggcctttgtgag

SEQ ID NO: 68 - DNA, 373 bp, Eremothecium gossypii
gtctgggtgcacgacacctgacctccgccccgcgggcttcctgttttcgccgggcgcggcacatggtgcggcttcctccgacaggaagccgggccgccggacgcgcacgtcagaggcgtcaccagggcaaatgggtggaagcgaagggaactacgacgaacggtcagcacccctggggcccccacgctcgcaccacagccgctgcgcgtgggcgtgaaaaattttacctgcgggctctccttacgatctcctattttatttcctggggggcagtcgaaatctatataagagggccccgggacgcacaacgggaggactctggtggagcgaccaggagtttgaattaattcagtccacacatacacaccgcaca

SEQ ID NO: 69 - protein, 525 aa, Eremothecium gossypii
MAAVEQVSSVFDTILVLDFGSQYSHLITRRLREFNVYAEMLPCTQKISELGWKPKGVILSGGPYSVYAADAPHVDRAVFELGVPILGICYGLQELAWIAGAEVGRGEKREYGRATLHVEDSACPLFNNVDSSTVWMSHGDKLHALPADFHVTATTENSPFCGIAHDSKPIFGIQFHPEVTHSSQGKTLLKNFAVEICQAAQTWTMENFIDTEIQRIRTLVGPTAEVIGAVSGGVDSTVAAKLMTEAIGDRFHAILVDNGVLRLNEAANVKKILGEGLGINLTVVDASEEFLTKLKGVTDPEKKRKIIGNTFIHVFEREAARIQPKNGEEIEFLLQGTLYPDVIESISFKGPSQTIKTHHNVGGLLDNMKLKLIEPLRELFKDEVRHLGELLGISHELVWRHPFPGPGIAIRVLGEVTKEQVEIARKADHIYIEEIRKAGLYNKISQAFACLLPVKSVGVMGDQRTYDQVIALRAIETTDFMTADWYPFEHEFLKHVASRIVNEVEGVARVTYDITSKPPATVEWE

SEQ ID NO: 70 - DNA, 1578 bp, Eremothecium gossypii
atggctgctgttgaacaagtttctagcgtgtttgacaccattttggtgctggacttcgggtcccagtactcgcatctgatcacgcggcggctgcgtgagtttaatgtgtacgcggagatgcttccgtgtacgcagaagatcagcgagctgggctggaagccaaagggtgtgattttgtcaggcgggccgtactccgtgtacgcggcagatgctccgcacgtggaccgggcggtgttcgagttgggcgttccaattctgggcatctgctacgggctacaggagcttgcgtggatagccggcgcagaggtggggcgcggcgagaagcgcgagtacgggcgcgcgacgctgcacgtggaggacagcgcgtgcccgctgttcaacaacgtggacagcagcacggtgtggatgtcgcacggtgacaagctgcacgcactacctgcggatttccacgtcactgcgacgacggagaactctcctttctgcgggattgcacacgactcgaagccaatcttcgggatccagttccaccctgaggtgacgcactcctcgcaggggaagacgttgctgaagaactttgcggtggagatctgccaggccgcgcagacctggacgatggaaaacttcattgacaccgagatccagcggatccggacccttgtgggccccaccgcggaagtcatcggtgctgtgtccggcggtgtcgactcgaccgtcgctgcgaagctgatgaccgaggccatcggcgaccggttccacgcgatcctggtcgacaacggtgttctgcgcctcaacgaagcggccaatgtgaagaaaatcctcggcgagggcttgggcatcaacttgactgttgttgacgcctccgaagagttcttgacgaagctcaagggcgtcacggaccctgagaagaagagaaagatcatcggtaacaccttcattcatgtttttgagcgcgaggcagccaggatccagcctaagaacggcgaggagattgagttcctgttgcagggtaccctataccctgacgttatcgagtccatttcctttaagggcccatctcagacgatcaagacccaccataacgtcggtggtcttttggacaacatgaaactgaagctcattgagcctttgcgcgagcttttcaaggacgaggtgagacacctgggagaactattggggatctcccacgagttggtctggagacatccgttcccaggcccaggtatcgccatccgtgtgctaggcgaggtcaccaaggagcaggtggagattgccagaaaggcagaccacatctacatcgaggagatcaggaaagcaggtctatacaacaagatttctcaagcttttgcttgcttgctgcctgttaagtctgtgggtgtcatgggtgaccagagaacctacgaccaggtcattgctctaagagcaattgagaccacggacttcatgactgccgactggtatccatttgagcacgaattcttgaagcatgtcgcatcccgtattgttaacgaggttgaaggtgttgccagagtcacctacgacataacttctaagcctccagctaccgttgaatgggaataa

SEQ ID NO: 71 - protein, 433 aa, Eremothecium gossypii
MVNVVLGSQWGDEGKGKLVDLLVSKYDIVARSAGGNNAGHTIVVGGIKYDFHMLPSGLVNPNCQNLIGNGVVIHVPSFFGELEQLEAKGLRDARGRLFISSRAHLVFDFHQRTDKLRELELSGKSKDGKNIGTTGKGIGPTYSTKASRSGLRVHHLVSEQPEAWAEFETKYRRLLETRQQRYGPFEHDAEEELARYRRYREELRPFVVDSVVFMHNAIQQKKKILVEGANALMLDIDFGTYPYVTSSSTGIGGVLTGLGIPPRCIDEIYGVVKAYTTRVGEGPFPTEQLNEAGDKLQTIGAEYGVTTGRKRRCGWLDLVVLKYSTLINGFTSLNITKLDVLDTFKEIKVGISYSLNGKKLDLFPEDLLVLSKVDVEYVTLPGWDEDITKISRYEDLPENAKSYLKFIEDFVGVPVEWVGTGPGRESMLHKEVS

SEQ ID NO: 72 - DNA, 1302 bp, Eremothecium gossypii
atggtcaacgtcgttctagggtcacagtggggtgacgaaggtaagggcaagctcgtggatttgctggtaagcaagtatgacattgtagcgcggtccgccgggggcaataatgctggacacacaattgttgtggggggaatcaagtacgacttccacatgttgccttcggggctggtcaaccctaattgccagaacttgatcggcaatggtgtggttattcacgtgccgtcgtttttcggcgagttggaacagctagaggccaagggtctgcgggacgcgcgcgggcggcttttcatttcatcgcgggcgcatttggtgtttgacttccaccagcgtacggataagctccgggagctcgagttgtctgggaagtccaaggacggaaagaacatcggcactacaggaaaaggtattggtccaacctactccaccaaagcttcacgttctggacttcgtgtgcaccatctcgtcagcgagcagccagaggcttgggcggaatttgagacgaaataccgccgactactggagactagacaacaacgctatgggccctttgaacacgacgcagaagaggagcttgctcgttacagacgctaccgggaagagcttagaccgtttgttgtggactctgtagtcttcatgcacaatgcgattcagcagaaaaagaagattctggttgagggcgccaatgctctgatgctggatattgactttggcacttatccatacgtcacatcttcatcgactggcatcggtggtgtcctgacaggtttaggcatcccacctcgctgtatcgatgagatatatggtgtagtgaaagcatacacgacacgtgtgggcgagggtccattcccaacggagcaattgaacgaggcaggggacaaattgcagaccattggcgccgagtatggtgttactacaggtcgcaagcgccggtgtggctggctcgatctggttgtgctaaaatattctaccttgatcaatgggttcacaagtttgaatataactaagcttgatgttttagatacgttcaaagagatcaaggtggggatttcatactccctcaatggtaagaagcttgatctgttccctgaggatctgctcgtcctcagtaaggtggatgttgagtatgttactttaccaggatgggacgaggatatcaccaagatctcccgctacgaagatcttccagagaatgccaagagttacttgaaattcattgaggacttcgttggcgtacctgttgaatgggtaggtaccggtcctgggagggaaagcatgttgcacaaggaagttagttag

SEQ ID NO: 73 - protein, 522 aa, Eremothecium gossypii
MTYRDAATALEHLATYAEKDGLSVEQLMDSKTRGGLTYNDFLVLPGKIDFPSSEVVLSSRLTKKITLNAPFVSSPMDTVTEADMAIHMALLGGIGIIHHNCTAEEQAEMVRRVKKYENGFINAPVVVGPDATVADVRRMKNEFGFAGFPVTDDGKPTGKLQGIITSRDIQFVEDETLLVSEIMTKDVITGKQGINLEEANQILKNTKKGKLPIVDEAGCLVSMLSRTDLMKNQSYPLASKSADTKQLLCGAAIGTIDADRQRLAMLVEAGLDVVVLDSSQGNSVFQINMIKWIKETFPDLQVIAGNVVTREQAASLIHAGADGLRIGMGSGSICITQEVMACGRPQGTAVYNVTQFANQFGVPCIADGGVQNIGHITKAIALGASTVMMGGMLAGTTESPGEYFFRDGKRLKTYRGMGSIDAMQKTDVKGNAATSRYFSESDKVLVAQGVTGSVIDKGSIKKYIPYLYNGLQHSCQDIGVRSLVEFREKVDSGSVRFEFRTPSAQLEGGVHNLHSYEKRLFD

SEQ ID NO: 74 - DNA, 1730 bp, Eremothecium gossypii
atgacttacagagacgcagccacggcactggagcacctggcgacgtacgccgagaaggacgggctgtccgtggagcagttgatggactccaagacgcggggcgggttgacgtacaacgacttcctggtcttgccgggcaagatcgacttcccatcgtcggaggtggtgctgtcgtcgcgcctgaccaagaagatcaccttgaacgcgccgtttgtgtcgtcgccgatggacacggtgacggaggccgacatggcgatccacatggcgctcctgggcggcatcgggatcatccaccacaactgcactgcggaggagcaggcggagatggtgcgccgggtcaagaagtacgaaaacgggttcatcaacgcccccgtggtcgtggggccggacgcgacggtggcggacgtgcgccggatgaagaacgagtttgggtttgcaggatttcctgtgacaggtatgttagagtggcacgcggggctgcacgctgggatgatgatcataaatcaataactttcgttctactgactgcgatcaaacgatcgtgtagacaccttttactctgaccgcagacgtgcagcgcctttttggcaggaacatgtactaacacatcagcagatgatggcaagccgaccgggaagctgcaggggatcatcacgtcccgtgacatccagtttgtcgaggacgagaccctgcttgtgtctgagatcatgaccaaggacgtcatcactgggaagcagggcatcaacctcgaggaggcgaaccagatcctgaagaacaccaagaagggcaagctgccaattgtggacgaggcgggctgcctggtgtccatgctttcgagaactgacttgatgaagaaccagtcctacccattggcctccaagtctgccgacaccaagcagctgctctgtggtgctgcgatcggcaccatcgacgcggacaggcagagactggcgatgctggtcgaggccggtctggacgttgttgtgctagactcctcgcagggtaactcggtcttccagatcaacatgatcaagtggatcaaggagaccttcccagacctgcaggtcattgctggcaacgtggtcaccagagagcaggctgccagcttgatccacgccggcgcagacgggttgcgtatcggtatgggctctggctccatctgtatcactcaggaggtgatggcctgtggtagaccacagggtaccgctgtctacaacgtcacgcagttcgccaaccagtttggtgtgccatgtattgctgacggtggtgtccagaacatcgggcacattaccaaagctatcgctcttggcgcgtccaccgtcatgatgggcggtatgctggcaggcactacagagtctccaggcgagtacttcttcagggacgggaagagactgaagacctacagaggtatgggctccatcgacgccatgcaaaagactgatgtcaagggtaacgccgctacctcccgttacttctctgagtctgacaaggttctggtcgctcagggtgttactggttctgtgatcgacaagggctccatcaagaagtacattccatatctgtacaatggtctacagcactcgtgccaggatatcggtgtgcgctctctagtggagttcagagagaaggtggactctggctcggtcagatttgagttcagaactccatctgcccagttggagggtggtgtgcacaacttgcactcctacgagaagcgcctatttgactga

SEQ ID NO: 75 - DNA, 9536 bp, Artificial Sequence
  plasmid pFaa1,4
ggaagctccaccccggttgataatcagaaaagccccaaaaacaggaagattgtataagcaaatatttatttaaatcctgtgaagataaccaaccaacttaggattgattccacaatccagatttgttactatatgttatatgatcacttttgccaagtcccagttcaatttcatcattctgcgaaaatgttacccgataggccagtcattcgtttgcctaattgtatttttaacctgttctaatcattcagggattattttatattattaccaccatttcgtcacaactcagccaacagttcacactattcttcttccagtggcactcacaacttgcaataattctggtattactgcggctttggactgctggaggaagtaagctagcttatcacctcgaataacttcgtataatgtatgctatacgaagttattaggtagatcgatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttatgctctagagctgtaaaagtcaaatccaacaaaatccggctatcgggagcggtgcgcgcccaacaaacctggacgccgggcccgtgcgacgcagcgcgacgcagcgcgaccgcgcagtacgtagacgcagctactgccacttccaccccacctcccaatatctggtccccgactcccccctcgcaccgcggcgacacagacttcgggctttctcaaaaaaggtatataacagcacccggctttacagctggcaggcgaggtcccggttgtcactacagccgaggcgctacacatacgcacagcgcaggttcctgtcacggcgaggcgcacatttatataaacacacaccagcgcgctcgagcacggacaaaaagagagcacagcaatgaagtcggccagtgttatagtaggagagcccgcagggcctcacgagacggcgccacggcgcaactccaagtgcccggatgcggtcgtggagcggccgctggggttcagctgcaacacggtatatgagtttgcgttggaggcgatggagcgcggcgggcggcagcgcgcgatggggtggcgggagacggtggagatccacgaggaccgcaagatggtgacgaaggtggtggacggcaaggagacggaggtggagaagacgtggttgtactacgagatgggcccgtaccagtacgtgacgtacgaccagctgcacgtggagatgcacgactacgggcgggggatggtgaagatggggctccagccgggcggcgaggaccgcttgcacattttcggcgcgacgtcgcaccggtggatgcggacgttcttggcagcgcagtcgcaggccatcacggtggtgacggcatacgacacgttgggcgagagcggcttgatctactcgctccagcagacggggtcgaaggcgatcttcgtggacaacaacctcttggagaagttggtgaagccggtgcaggagatcccggacttgaagtacgtgatccacgcggacccgctcgacccggaggacaagcgctacggcggccggatgtactctgacgcgcagaaggcgatcgaccgcatgaaggaggttcggccggacatcgaggtttacagcatggacgaggtcgtggagctcggatcgctctgccgggactcgatttttgtgcaccggccacgcaagaaggaccttgcgtgcatcatgtacacctcgggctcgacaggtgacccgaagggtgtgtcgttgacccacgctaacatcgtggcgggcattggcggtgtttccgttgtgatcaaccgcgcgattgtgaagcctgacgatcgtgtcatcgcgttcttgccgcttgcgcatatttttgagcttgtgttcgagttgacctgtctctactggggcgccttaattggctacggctccgtcaagacgttgagcgaggcttcggtccgcaactgtaagggcgacatgaaggagttccggccgtccgtcatggtcggtgtcgcagctgtctgggagggtgtcaggaaggctattgttgcgcaggtcactaagttgcctccgttcaagcaaaagatattctgggcggcctaccacaccaagctacgcatgaagaagtgccacattccaggcggcgatctaataggaagcatgatctttaagaaggtgcgtgagaccactggtggcaaccttcgctacatcttgaatggtggctctccattgtcgcgggatacgcaagtttttatttccaacttgatttgccccgtgttgattggttacggcttaacggagactgtggcgaatggctgtatagtgcctccacaccacttcaagtacggggttgtgggagacattcttggttctctaacggtcaaattggtcgatgtcgaggagctcggctatcttgccaagaacaaccagggtgagctctgggtcaagggccccgccgtgtttaaagactacttgcagaacgaggccgagaccgctgccgctttggaagacgggtggttcaagactggtgacattgccgaatggacgaagaagggtcaattgcgtttgatcgaccgtaagaagaaccttgtcaagacgttgaatggtgaatacatcgctttggagaagttggagtgcatctacagatccaacaagtatgtggccaacatctgtgtctatgctgaccagaccaaggtcaagccaattgcgattgtggttccaaacgtcaatgctgtcaccgatttggccatctcattggggttgatcaaggacggttgcgaggtacgtgatgtttatgatagcaaaaagttgaagaaggtgatcttggacgacatgcataaaactgccaagggccagggattgggtggtattgagttgattcttgggttcgtgatcttcgatgatgagtggaccccacagaatggctatgtcacctctgcgcagaagctacagagaagaaagatcttgtctgcagtgcagtcagaagttgacgcactatatgccgcgaactcttaaatcaatccatcctgagtgaagggatcaccatatgattaatgccgttgtacttttagttttgaacgaatagtttattagctaaaatccaaaaaaaaaaaaccgagacctccgaagatattgtcactgccagtaacgctcttcatgctcatggttaatgatggtatgtgggcacagctggtcttatgaaattgcatatgtgatactttaccgtatggtcatggagccattcctaccataagcctcacgtgttggcgtgagtaacctctctgctattcaagggaattgctggtggcggcatgtcacccttatggtctaccattgtaatcatgtgttctacggcggaataaaagggtttatacagccgctattgcatagctgcagtagcaattggcggtattgcttttaatatcggtctctaatttgagggaactggttttctcgttattgtgtgtggacacctgcttcgaatcttactgctgcttgttacaatatgaaatctcttacactttgttataaacactattgtcctcgggggaagaagtattggaatacatattattacgtatatgcacttccgtcaatattagcttcacaattctaaccgaataaagattcgtaactattgtagcttccagtaattgattccagtatttaaatggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatg

SEQ ID NO: 76 - DNA, 22 bp, Artificial Sequence
  primer p18
ctgtgatcgaggatcatctttc

SEQ ID NO: 77 - DNA, 22 bp, Artificial Sequence
  primer p19
gctatgtcacctctgcgcagaa

SEQ ID NO: 78 - DNA, 22 bp, Artificial Sequence
  primer p20
gtgacttcacttgccactaatc

SEQ ID NO: 79 - DNA, 20 bp, Artificial Sequence
  primer p21
gtggcagtagctgcgtctac

(19) United States
(12) Patent Application Publication
(10) Pub. No.: US 2016/0298160 A1
(43) Pub. Date: Oct. 13, 2016

(54) OVER-EXPRESSION OF A FATTY ACID TRANSPORTER GENE AND OF GENES ENCODING ENZYMES OF THE BETA-OXIDATION PATHWAY FOR HIGHER PRODUCTION OF RIBOFLAVIN VIA FERMENTATION OF EREMOTHECIUM

(71) Applicant: BASF SE, Ludwigshafen (DE)

(72) Inventors: Birgit HOFF, Pfungstadt (DE); Andrea MOLT, Grünstadt (DE); Stefan HAEFNER, Speyer (DE); Oskar ZELDER, Speyer (DE)

(21) Appl. No.: 15/100,376

(22) PCT Filed: Dec. 4, 2014
(86) PCT No.: PCT/EP2014/076580

§ 371 Date: May 31, 2016

(30) Foreign Application Priority Data
Dec. 9, 2013 (EP) 13196262.3

(51) Int. Cl. C12P 25/00; C12N 15/80

(52) U.S. Cl. CPC C12P 25/00; C12N 15/80

(57) ABSTRACT

The present invention relates to a method of producing riboflavin in a genetically modified organism of the genus Eremothecium, wherein said genetic modification is linked to the fatty acid uptake and/or beta-oxidation pathway of said organism, comprising growing said organisms in a culture medium and isolating riboflavin from the culture medium. The invention further relates to a method of providing a riboflavin accumulating organism belonging to the genus Eremothecium by genetically modifying said organism, to organisms obtained by such a method, as well as the use of such genetically modified organisms for increasing the accumulation of riboflavin.

### FIELD OF THE INVENTION

The present invention relates to a method of producing riboflavin in a genetically modified organism of the genus Ashbya or as also named Eremothecium, wherein said genetic modification is linked to the fatty acid uptake and/or beta-oxidation pathway of said organism, comprising growing said organisms in a culture medium and isolating riboflavin from the culture medium. The invention further relates to a method of providing a riboflavin accumulating organism belonging to the genus Eremothecium by genetically modifying said organism, to organisms obtained by such a method, as well as the use of such genetically modified organisms for increasing the accumulation of riboflavin.

### BACKGROUND

Riboflavin is produced by all plants and a number of microorganisms such as fungi, yeasts or bacteria. Riboflavin is an essential component of the cellular metabolism since it serves as a precursor of the flavin coenzymes flavinmononucleotide (FMN) and flavin adenine dinucleotide (FAD), which are important electron carriers in redox reactions and participate in light sensing, DNA protection etc. Higher eukaryotes including humans cannot synthesize riboflavin, so that riboflavin has obtained the status of vitamin (vitamin B2). Vitamin B2 deficiency in humans results in inflammations of the oral and pharyngeal mucous membranes, itching and inflammation in cutaneous folds and skin damage, conjunctivitis, reduced visual acuity and corneal opacification. In babies and children, inhibition of growth and weight loss may occur. Therefore, riboflavin has to be supplemented to human or animal diets. It is thus added to feed and food stuff and may also be used as food coloring, e.g. in mayonnaise or ice cream.

Riboflavin may be produced chemically or microbially. Chemical approaches to synthesize riboflavin are based on a multi-step process using as starting for example material D-ribose. Microbial approaches to produce riboflavin are based on several microorganisms' potential to naturally synthesize riboflavin, in particular in the presence of suitable raw material such as vegetable oils. Microorganisms which are known as riboflavin producers include Candida famata, Bacillus subtilis and Eremothecium species (Stahmann, 2010, Industrial Applications, The Mycota X, 2^{nd} ed., Springer, Berlin, Heidelberg, page 235-247).

In particular, filamentous hemiascomycete fungi of the genus Eremothecium (previously Ashbya; belonging to the family of Saccharomycetaceae) were identified as potent riboflavin producers. In the last years, the riboflavin producing species Eremothecium gossypii has intensively been researched and analyzed and its genome has been sequenced.

In E. gossypii (Ashbya gossypii), the riboflavin production phase was found to be linked to a strong increase in transcription of several riboflavin biosynthesis genes (e.g. RIB genes RIB 1, 2, 3, 4, 5 and 7). Accordingly, riboflavin producing strains have been developed which involve the over-expression of these genes, e.g. by integration of additional copies, as outlined in WO 95/26406 or WO 99/61623.

Furthermore, the riboflavin biosynthesis pathway of Eremothecium could be clarified (Fischer and Bacher, 2005, Nat Prod Rep, 22, pages 324-350). The production of riboflavin in Eremothecium could, based on a better understanding of the biosynthesis pathways, be increased by the over-expression of GLY1 encoding a thereonine aldolase and the disruption of the gene SHM encoding the cytosolic serine hydroxymethyltransferase which both interfere with the GTP metabolism (see also FIGS. 1 and 2) which is essential for the production of riboflavin (Stahmann, 2010, Industrial Applications, The Mycota X, 2^{nd} ed., Springer, Berlin, Heidelberg, 235-247). A further important regulatory gene, which was found to influence the production of riboflavin via interfering with the phospho-ribosylamine synthesis is ADE4 encoding a phosphoribosyl-pyrophosphate am idotransferase, which can be provided as feed-back resistant version (Jimenez et al., 2005, Appl Environ Microbol, 71, 5743-5751). The modified steps of the riboflavin pathway identified so far are essentially confined to the terminal steps of the riboflavin biosynthesis.

However, despite these developments the synthesis efficiency and the amount of produced riboflavin, in particular in the genetic background of Eremothecium fungi, are still non-optimal, while the demand for food- and feed-grade riboflavin is ever increasing.

There is hence a need for means and methods allowing to further improve the production and accumulation or riboflavin in suitable organisms such as fungi of the genus Eremothecium.

### OBJECTS AND SUMMARY OF THE INVENTION

The present invention addresses this need and presents a method of producing riboflavin in a genetically modified organism of the genus Eremothecium wherein said modifications are linked to the fatty acid uptake and the beta-oxidation and which allow an increase of the riboflavin production compared to an organism not having the genetic modification which is cultured under the same conditions as the genetically modified organism.

Accordingly, the present invention provides in a first aspect a method of producing riboflavin in a genetically modified organism of the genus Eremothecium, wherein said genetic modification is linked to the fatty acid uptake and/or beta-oxidation pathway of said organism, comprising growing said organisms in a culture medium and isolating riboflavin from the culture medium. The present invention provides in particular a method wherein said genetic modification results at least in the increase of the AGOS_ACL174Wp (Fat1) activity and/or the increase of the AGOS_AER358Cp (Pox1) activity and/or the increase of the AGOS_AGL060Wp (Fox2) and/or the AGOS_AFR302Wp (Pot1/Fox3) and/or the AGOS_ABL018C (Faa 1,4) activity of said organism.

The inventors surprisingly found that by increasing the activity of a component of the long-chain fatty acid transport apparatus of Eremothecium an increase of the production or accumulation of riboflavin could be achieved. Especially, they found that by increasing the activity of AGOS_ACL174Wp (Fat1), which is a component of the long-chain fatty acid transport apparatus of Eremothecium and which is also believed to be involved in the vary long-chain fatty acid activation a significant increase of the production or accumulation of riboflavin could be achieved. The inventors further found that by increasing the activity of an enzymatic activity involved in the beta-oxidation pathway of Eremothecium a significant increase of the production or accumulation of riboflavin could be achieved. Particularly, the inventors found that by increasing the activity of AGOS_AER358Cp (Pox1), i.e. a peroxisomal oxidase involved in the beta-oxidation pathway of Eremothecium a significant increase of the production or accumulation of riboflavin could be achieved. Furthermore, they surprisingly found that by increasing the activity of AGOS_AGL060Wp (Fox2) and the AGOS_AFR302Wp (Pot1/Fox3), i.e. of a hydratase/dehydrogenase and a 3-ketoacyl-CoA thiolase, respectively, of the beta-oxidation pathway of Eremothecium a significant increase of the production or accumulation of riboflavin becomes possible. The inventors also found that by increasing the activity of AGOS_ABL0180 (Faa 1,4), i.e. a long-chain acyl-CoA synthetase which mediates esterification of fatty acids and thereby regulates fatty acid transport, a significant increase of the production or accumulation of riboflavin could be achieved.

These results are unexpected in so far as the enzymatic activities which were hitherto believed to have an influence on the production efficiency or amount of riboflavin produced are typically associated with the terminal steps of riboflavin biosynthesis or with anaplerotic reactions leading to glycine or GTP being used as intermediates for the riboflavin synthesis (see also FIG. 1), while early biosynthetic reactions such as beta-oxidation steps or fatty acid transport activities have not yet been described as relevant steps for the production of riboflavin, in particular in the context of Eremothecium fungi.

The use of Eremothecium additionally provides several advantages over the use of other microorganisms. The representative species Eremothecium gossypii has intensively been researched and analyzed, its genome has been sequenced and there are several molecular tools available allowing for genetic manipulation and engineering. Furthermore, it could be demonstrated that Eremothecium is able to grow in different oil sources and oil-containing wastes (Park et al., 2004, J Amer Oil Chem Soc, 81: 57-62), and glycerol (Ribeiro et al., 2012, J Basic Microbiol, 52: 582-589) thus allowing for a high efficiency use of these cheap energy sources as starting material for the production of riboflavin.

In a related aspect the present invention relates to a method of producing riboflavin in an organism of the genus Eremothecium which is genetically modified to increase the activity of at least one protein linked to the fatty acid uptake and/or the beta oxidation pathway compared to an organism not having said genetic modification which is cultured under the same conditions as the genetically modified organism, said method comprising growing said organism in a suitable culture medium and isolating riboflavin from the culture medium.

In a further aspect the present invention relates to a method of providing a riboflavin accumulating organism belonging to the genus Eremothecium by genetically modifying said organism, wherein said genetic modification is linked to the fatty acid uptake and/or beta-oxidation pathway of said organism.

In yet another aspect the present invention relates to a riboflavin accumulating organism belonging to the genus Eremothecium, which is genetically modified, wherein said genetic modification is linked to the fatty acid uptake and/or beta-oxidation pathway of said organism.

In a related aspect the present invention relates to a riboflavin accumulating organism belonging to the genus Eremothecium, which is genetically modified to increase the activity of at least one protein linked to the fatty acid uptake and/or the beta oxidation pathway in said organism compared to an organism not having the genetic modification which is cultured under the same conditions as the genetically modified organism.

In a particularly preferred embodiment of the method or organism as defined above, said genetic modification results at least in the increase of the AGOS_ACL174Wp (Fat1) activity and/or the increase of the AGOS_AER358Cp (Pox1) activity and/or the increase of the AGOS_AGL060Wp (Fox2) and/or the AGOS_AFR302Wp (Pot1/Fox3) and/or the AGOS_ABL0180 (Faa 1,4) activity of said organism.

In a further preferred embodiment of the method or organism as defined above, the genetically modified organism is capable of accumulating at least 5 to 10% more riboflavin than a comparable organism without the genetic modification.

In a further preferred embodiment of the present invention said increase of the AGOS_ACL174Wp (Fat1) activity is due to the over-expression of the AGOS_ACL174W gene (fat1); and/or said increase of the AGOS_AER358Cp (Pox1) activity is due to the over-expression of the AGOS_AER358C gene (pox1); and/or said increase of the AGOS_AGL060Wp (Fox2) activity and/or the AGOS_AFR302Wp (Pot1/Fox3) activity is due to the over-expression of the AGOS_AGL060W gene (fox2) and the AGOS_AFR302W gene (pot1/fox3) and/or said increase of the AGOS_ABL018C (Faa 1,4) activity is due to the over-expression of the AGOS_ABL0180 gene (faa 1,4).

In another preferred embodiment of the present invention said over-expression of the AGOS_ACL174W gene (fat1), the AGOS_AER358C gene (pox1), the AGOS_AGL060W gene (fox2), the AGOS_ABL018C gene (faa 1,4) and/or the AGOS_AFR302W gene (pot1/fox3) is conveyed by a strong promoter, preferably the GPD promoter, or by the provision of at least a second copy of the AGOS_ACL174W gene (fat1), the AGOS_AER358C gene (pox1), the AGOS_AGL060W gene (fox2), the ABL018C gene (faa 1,4) and/or the AGOS_AFR302W gene (pot1/fox3) in the genome of the organism. In specifically preferred embodiments, said strong promoter is a constitutive promoter. Optionally, the promoter may also be a strong regulable promoter.

In another preferred embodiment of the present invention said fat1 gene comprises a nucleic acid sequence selected from the group consisting of:

(a) the nucleic acid sequence according to SEQ ID No. 2 or a functional part thereof;
(b) a nucleic acid sequence encoding the polypeptide according to SEQ ID No. 1 or a functional part or variant thereof;
(c) a nucleic acid sequence which as a result of the degeneracy of the genetic code can be derived from the nucleic acid sequence according to SEQ ID No. 2; and
(d) a nucleic acid sequence having at least 70% sequence identity to the nucleic acid sequence according to SEQ ID No. 2.

In another preferred embodiment of the present invention said pox1 gene comprises a nucleic acid sequence selected from the group consisting of:

(a) the nucleic acid sequence according to SEQ ID No. 6 or a functional part thereof;
(b) a nucleic acid sequence encoding the polypeptide according to SEQ ID No. 5 or a functional part or variant thereof;
(c) a nucleic acid sequence which as a result of the degeneracy of the genetic code can be derived from the nucleic acid sequence according to SEQ ID No. 6; and
(d) a nucleic acid sequence having at least 70% sequence identity to the nucleic acid sequence according to SEQ ID No. 6.

In another preferred embodiment of the present invention said fox2 gene comprises a nucleic acid sequence selected from the group consisting of:

(a) the nucleic acid sequence according to SEQ ID No. 8 or a functional part thereof;
(b) a nucleic acid sequence encoding the polypeptide according to SEQ ID No. 7 or a functional part or variant thereof;
(c) a nucleic acid sequence which as a result of the degeneracy of the genetic code can be derived from the nucleic acid sequence according to SEQ ID No. 8; and
(d) a nucleic acid sequence having at least 70% sequence identity to the nucleic acid sequence according to SEQ ID No. 8.

In another preferred embodiment of the present invention said faa1/faa4 gene comprises a nucleic acid sequence selected from the group consisting of:

(a) the nucleic acid sequence according to SEQ ID No. 4 or a functional part thereof;
(b) a nucleic acid sequence encoding the polypeptide according to SEQ ID No. 3 or a functional part or variant thereof;
(c) a nucleic acid sequence which as a result of the degeneracy of the genetic code can be derived from the nucleic acid sequence according to SEQ ID No. 4; and
(d) a nucleic acid sequence having at least 70% sequence identity to the nucleic acid sequence according to SEQ ID No. 4.

In another preferred embodiment of the present invention said pot1/fox3 gene comprises a nucleic acid sequence selected from the group consisting of:

(a) the nucleic acid sequence according to SEQ ID No. 10 or a functional part thereof;
(b) a nucleic acid sequence encoding the polypeptide according to SEQ ID No. 9 or a functional part or variant thereof;
(c) a nucleic acid sequence which as a result of the degeneracy of the genetic code can be derived from the nucleic acid sequence according to SEQ ID No. 10; and
(d) a nucleic acid sequence having at least 70% sequence identity to the nucleic acid sequence according to SEQ ID No. 10.

In another preferred embodiment of the present invention said genetically modified organism as defined herein above comprises at least one additional genetic modification. In a particularly preferred embodiment said additional genetic modification results in the alteration of at least one activity selected from the group comprising:

### (i) GLY1;

### (ii) SHM2;

(iii) ADE4;

### (iv) PRS 2, 4

### (v) PRS 3;

### (vi) MLS1;

(vii) BAS1;
(viii) RIB 1;

### (ix) RIB 2;

### (x) RIB 3;

### (xi) RIB 4;

(xii) RIB 5;
(xiii) RIB 7
(xiv) ADE12;

### (xv) GUA1; and

(xvi) IMPDH.

In a further preferred embodiment said additional genetic modification results in at least one of the following alterations:

(i) the GLY1 activity is increased; and/or
(ii) the SHM2 activity is decreased or eliminated; and/or
(iii) the ADE4 activity is increased and/or provided as feedback-inhibition resistant version; and/or
(iv) the PRS 2, 4 activity is increased; and/or
(v) the PRS 3 activity is increased; and/or
(vi) the MLS1 activity is increased; and/or
(vii) the BAS1 activity is decreased or eliminated; and/or
(viii) the RIB 1 activity is increased; and/or
(ix) the RIB 2 activity is increased; and/or
(x) the RIB 3 activity is increased; and/or
(xi) the RIB 4 activity is increased; and/or
(xii) the RIB 5 activity is increased; and/or
(xiii) the RIB 7 activity is increased; and/or
(xiv) the ADE12 activity is decreased; and/or
(xv) the GUA1 activity is increased; and/or
(xvi) the IMPDH activity is increased.

In a further aspect the present invention relates to a use of the AGOS_ACL174W gene (fat1), the AGOS_AER358C gene (pox1), the AGOS_AGL060W gene (fox2), the AGOS_ABL018Cp gene (faa 1,4) and/or the AGOS_AFR302W gene (pot1/fox3) for increasing the accumulation of riboflavin in an organism of the genus Eremothecium.

In a preferred embodiment of said use, the AGOS_ACL174W gene (fat1), the AGOS_AER358C gene (pox1), the AGOS_AGL060W gene (fox2), the AGOS_ABL018Cp gene (faa 1,4) and/or the AGOS_AFR302W gene (pot1/fox3) is over-expressed via a strong promoter, preferably the GPD promoter, or by the provision of at least a second copy of the AGOS_ACL174W gene (fat1), the AGOS_AER358C gene (pox1), the AGOS_AGL060W gene (fox2), the AGOS_ABL018Cp gene (faa 1,4) and/or the AGOS_AFR302W gene (pot1/fox3) in the genome of the organism. In specifically preferred embodiments, said strong promoter is a constitutive promoter. Optionally, the promoter may also be a strong regulable promoter.

In a further preferred embodiment of said use, said fat1 gene comprises a nucleic acid sequence selected from the group consisting of:

(a) the nucleic acid sequence according to SEQ ID No. 2 or a functional part thereof;
(b) a nucleic acid sequence encoding the polypeptide according to SEQ ID No. 1 or a functional part or variant thereof;
(c) a nucleic acid sequence which as a result of the degeneracy of the genetic code can be derived from the nucleic acid sequence according to SEQ ID No. 2; and
(d) a nucleic acid sequence having at least 70% sequence identity to the nucleic acid sequence according to SEQ ID No. 2.

In another preferred embodiment of said use, said pox1 gene comprises a nucleic acid sequence selected from the group consisting of:

(a) the nucleic acid sequence according to SEQ ID No. 6 or a functional part thereof;
(b) a nucleic acid sequence encoding the polypeptide according to SEQ ID No. 5 or a functional part or variant thereof;
(c) a nucleic acid sequence which as a result of the degeneracy of the genetic code can be derived from the nucleic acid sequence according to SEQ ID No. 6; and
(d) a nucleic acid sequence having at least 70% sequence identity to the nucleic acid sequence according to SEQ ID No. 6.

In another preferred embodiment of said use, said fox2 gene comprises a nucleic acid sequence selected from the group consisting of:

(a) the nucleic acid sequence according to SEQ ID No. 8 or a functional part thereof;
(b) a nucleic acid sequence encoding the polypeptide according to SEQ ID No. 7 or a functional part or variant thereof; and
(c) a nucleic acid sequence which as a result of the degeneracy of the genetic code can be derived from the nucleic acid sequence according to SEQ ID No. 8;
(d) a nucleic acid sequence having at least 70% sequence identity to the nucleic acid sequence according to SEQ ID No. 8.

In another preferred embodiment of said use, said faa1/faa4 gene comprises a nucleic acid sequence selected from the group consisting of:

(a) the nucleic acid sequence according to SEQ ID No. 4 or a functional part thereof;
(b) a nucleic acid sequence encoding the polypeptide according to SEQ ID No. 3 or a functional part or variant thereof;
(c) a nucleic acid sequence which as a result of the degeneracy of the genetic code can be derived from the nucleic acid sequence according to SEQ ID No. 4; and
(d) a nucleic acid sequence having at least 70% sequence identity to the nucleic acid sequence according to SEQ ID No. 4.

In another preferred embodiment of said use, said pot1/fox3 gene comprises a nucleic acid sequence selected from the group consisting of:

(a) the nucleic acid sequence according to SEQ ID No. 10 or a functional part thereof;
(b) a nucleic acid sequence encoding the polypeptide according to SEQ ID No. 9 or a functional part or variant thereof;
(c) a nucleic acid sequence which as a result of the degeneracy of the genetic code can be derived from the nucleic acid sequence according to SEQ ID No. 10; and
(d) a nucleic acid sequence having at least 70% sequence identity to the nucleic acid sequence according to SEQ ID No. 10.

In another aspect the present invention relates to the use of an organism as defined herein above for the production of riboflavin.

In a particularly preferred embodiment of the method, use or organism as defined herein above, said organism belonging to the genus Eremothecium is of the species Eremothecium ashbyi, Eremothecium coryli, Eremothecium cymbalariae, Eremothecium gossypii, Eremothecium sinecaudum or Eremothecium sp. CID1339.

In a final aspect, the present invention relates to a riboflavin product from at least one organism as defined as define herein above.

### FIGURE LEGENDS

FIG. 1 depicts the metabolic flux towards riboflavin (vitamin B2). Riboflavin is produced from fatty acids through the glyoxylate cycle, gluconeogenesis, the pentose phosphate pathway and the purine and riboflavin synthetic pathways.

FIG. 2 shows the terminal steps of the riboflavin (vitamin B2) biosynthesis. Riboflavin is synthesized from the GTP and ribulose-5-phosphate as precursors involving six enzymatic activities. The corresponding enzymes are encoded by the RIB genes (RIB1, 2, 3, 4, 5 and 7).

FIG. 3 depicts a simplified diagram of the fatty acid biosynthesis and degradation in E. gossypii including parts of the beta oxidation pathway. Dashed arrows indicate a multi-step pathway.

FIG. 4 depicts a map of the plasmids pGPDp-FAT1 generated for over-expression of the fatty acid transporter gene FAT1. Abbreviations: G418: KanMX resistance marker, HomA and HomB: genome integration sites, loxP: recombination site for CRE recombinase, ORI-EC: origin of replication for Escherichia coli, ampR: ampicillin resistance gene, BseRI/BsgI: restriction sites.

FIG. 5 shows a map of plasmid pGPDp-POX1 generated for over-expression of the gene POX1 encoding an enzyme of the beta-oxidation pathway. Abbreviations: G418: KanMX resistance marker, HomA and HomB: genome integration sites, loxP: recombination site for CRE recombinase, ORI-EC: origin of replication for Escherichia coli, kanR: kanamycin resistance gene, SwaI: restriction site.

FIG. 6 shows a map of plasmid pPOT1-FOX2 generated for over-expression of the genes POT1 and FOX2 encoding further enzymes of the beta-oxidation pathway. Abbreviations: G418: KanMX resistance marker, HomA and HomB: genome integration sites.

FIG. 7 shows the riboflavin yield in the E. gossypii engineered strains compared to the reference strain PS3. FIG. 7 A depicts the quantification of riboflavin production in two independent generated strains over-expressing the FAT1 gene under control of the E. gossypii GPD promoter. FIG. 7 B shows the quantification of riboflavin production in two independent generated strains over-expressing the POX1 gene under control of the E. gossypii GPD promoter. FIG. 7 C shows the quantification of riboflavin production in two independent generated strains containing a second copy of the POT1 and FOX2 genes. All experiments were performed as triplicate.

FIG. 8 shows a map of plasmid pFAA1,4 generated for over-expression of the gene FAA1,4 encoding an enzyme of the beta-oxidation pathway. Abbreviations: G418: KanMX resistance marker, HomA and HomB: genome integration sites, loxP: recombination site for CRE recombinase, ORI-EC: origin of replication for Escherichia coli, ampR: ampicillin resistance gene, URA3: gene encoding the orotidine 5′-phosphate decarboxylase for selection in S. cerevisiae, 2 μm ori: origin of replication for S. cerevisiae, SwaI: restriction site.

FIG. 9 shows the riboflavin yield in the E. gossypii engineered strains over-expressing either FAT1, POX1, FAA1/FAA4 or POT1 or FOX2 compared to the wild type strain ATCC10895. All experiments were performed in triplicate.

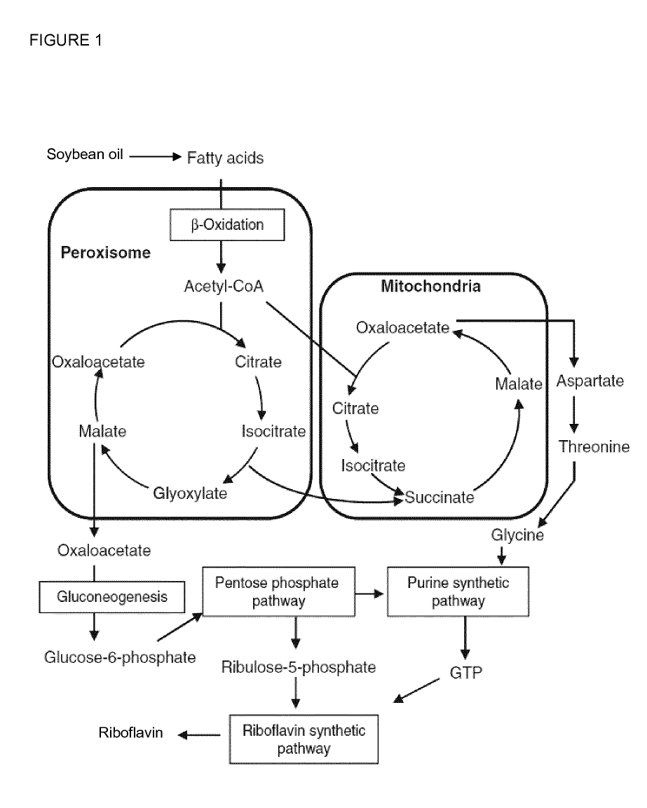

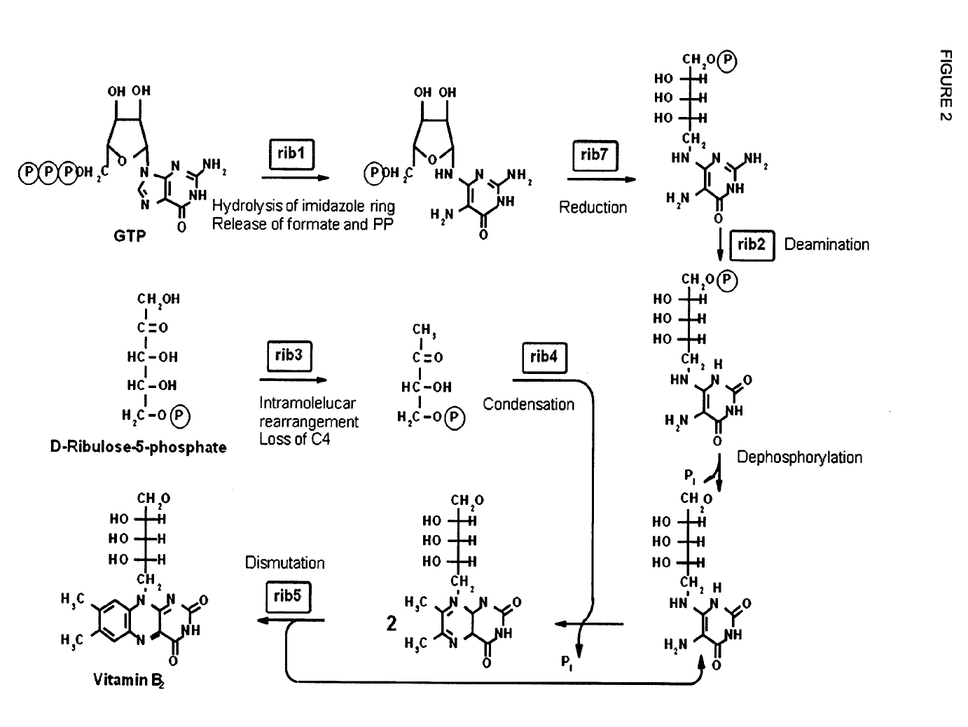

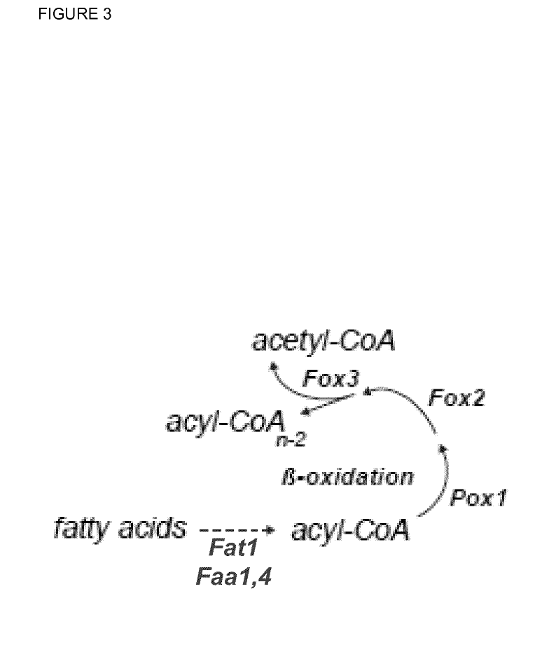

### DETAILED DESCRIPTION OF THE INVENTION

The present invention relates to improved means and methods allowing to produce riboflavin by using an organism belonging to the genus Eremothecium (previously Ashbya) which is genetically modified and wherein said modifications are linked to the fatty acid uptake and the beta-oxidation pathway.

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect a method of producing riboflavin in a genetically modified organism of the genus Eremothecium, wherein said genetic modification is linked to the fatty acid uptake and/or beta-oxidation pathway of said organism, comprising (i) growing said organisms in a culture medium, preferably in the presence of fatty acid oils; and optionally in the presence of non-lipid carbon sources; and (ii) isolating riboflavin from the culture medium.

The term “organism belonging to the genus Eremothecium” or “Eremothecium organism” as used herein means any organism belonging to the genus Eremothecium, which was previously known and/or is synonymous to the genus Ashbya. This group comprises at least the species Eremothecium ashbyi, Eremothecium coryli, Eremothecium cymbalariae, Eremothecium gossypii (previously Ashbya gossypii), Eremothecium sinecaudum and Eremothecium sp. CID1339. Further included are variants of these species, clones or modified organisms based on these species. The term “modified organism” as used herein refers to a modification of a wildtype species of Eremothecium by mutagenesis and selection and/or genetic engineering, or to a modification of an already genetically modified organism, e.g. an Eremothecium strain which was previously engineered to increase the production of riboflavin, or being modified or engineered for any other purpose. The term specifically includes Eremothecium species which were obtained by general mutagenesis approaches such as chemical or UV mutagenesis or disparity mutagenesis. In a preferred embodiment the organism of the genus Eremothecium is Eremothecium gossypii and in a more preferred embodiment it is Eremothecium gossypii of the strain ATCC 10895.

The term “an organism not having the genetic modification” as used herein refers to an organism which is not genetically modified to increase the activity of a protein linked to the fatty acid uptake and/or beta-oxidation pathway, in particular the AGOS_ACL174Wp (Fat1) activity and/or the AGOS_AER358Cp (Pox1) activity and/or the AGOS_AGL060Wp (Fox2) activity and/or the AGOS_AFR302Wp (Pot1/Fox3) activity and/or the AGOS_ABL0180 (Faa 1,4) activity, and which, apart from that, has the same genetic constitution as the genetically modified organism of the present invention, i.e. the only genetic difference to the genetically modified organism of the present invention is the genetic modification of the present invention. Hence, the organism not having the genetic modification is the parental strain into which the genetic modification is introduced within the invention and preferably it is Eremothecium gossypii of the strain ATCC 10895. The parental strain may not comprise any genetic modification or it may comprise genetic modifications other than those of the present invention.

The term “growing said organism in a culture medium” as used herein refers to the use of any suitable means and methods known to the person skilled in the art, which allows the growth of the organism as defined herein and which is suitable for the synthesis and/or accumulation of riboflavin. The growing may be carried out as batch process or in a continuous fermentation process. Preferably, the organism is grown in the presence of fatty acid oils and optionally in the presence of non-lipid carbon sources.

Methods for carrying out batch or continuous fermentation processes are well known to the person skilled in the art and are described in the literature. The culturing may be carried out under specific temperature conditions, e.g. between 15° C. and 45° C., preferably between 20° C. and 40° C. or 15° C. and 30° C., more preferably between 20° C. and 30° C. and most preferably at 28° C. In another embodiments the culturing may be carried out at a broad pH range, e.g., between pH 6 and pH 9, preferably between pH 6.5 and 8.5, more preferably between 6.7 and 7.5 and most preferably between 6.8 and 7.

The term “fatty acid oil” as used herein refers to waste oils, non-edible oils, or cheap seed oils. A preferred example of such an oil is soya bean oil or rapeseed oil. The fatty acid oil may be present in the culture medium in any suitable amount or concentration, e.g. in a concentration of 5% (v/v) to 40% (v/v), for instance in a concentration of 5%, 7.5%, 10%, 12.5%, 15%, 17.5% 20%, 22.5%, 25%, 27.5%, 30%, 32.5%, 35%, 37.5%, or 40%. Preferably a concentration of about 10% may be used.

The term “in the presence of non-lipid carbon sources” as used herein means that the culturing is carried out in the presence of nutrients which do not belong to the group of lipids. Preferably, the culturing may be performed in the presence of sugar nutrients, e.g. in the presence of glucose, sucrose, fructose etc.

In further specific embodiments, the culture medium may comprise additional substances. An example of such an additional substance is soybean flour. The soybean flour may preferably be provided in a concentration of 1% (w/v) to 5% (w/v), e.g. 1%, 2%, 3%, 4%, 5% (w/v). Soybean flour is a complex medium, which typically comprises proteins, carbohydrates and salts.

A further example of an additional substance is glycine. Glycine may preferably be provided in a concentration of 1% (w/v) to 5% (w/v), e.g. 1%, 2%, 3%, 4%, 5% (w/v).

In a very specific embodiment, the culture medium may comprise the following ingredient: yeast extract, soybean flour, glycine, sodium glutamate, KH₂PO₄, MgSO₄, DL-methionine, inositol, sodium formate, urea and rapeseed or soybean oil. In a particularly preferred embodiment, the culture medium may comprise ingredients in concentrations and amounts as described in the Examples below.

The wording “isolating riboflavin from the cells and culture medium” as used herein refers to any suitable method of extracting the riboflavin from the cells and separating the riboflavin from cell debris and ingredients of the culture medium. In a preferred embodiment, the isolation may be carried out as described in Stahmann, Industrial Applications, 2^{nd} edition, The Mycota X, M. Hofrichter (Ed.), Springer Verlag Berlin Heidelberg, 2010, pages 235 to 247.

The term “producing riboflavin” as used herein means that an Eremothecium organism is able to synthesize and accumulate riboflavin. The term “accumulate riboflavin” means that the synthesized riboflavin is stored intracellularly and/or is excreted into the surrounding medium, in both cases leading to an overall increase of the riboflavin concentration in the cell culture. The accumulation may, in specific embodiments, become discernible after a suitable isolation process in which all riboflavin produced by the cell, i.e. including intracellulary stored riboflavin and excreted riboflavin, is obtained. Such a process has been described herein above.

The production of riboflavin as meant in the context of the present invention typically differs from the synthesis of riboflavin in wildtype organisms, i.e. it refers to an overproduction of riboflavin in comparison to a wildtype strain of Eremothecium. A wildtype strain of Eremothecium typically produces about 50 to 100 mg riboflavin per liter cell culture, in particular under cell culture conditions as defined herein above, or in the Examples. The term “overproduction” as used herein refers to a production of riboflavin of more than about 50 to 100 mg/I of the cell culture. The term “riboflavin overproducing organism” or “riboflavin overproducing strain” accordingly refers to an Eremothecium organism or strain which produces more than about 50 to 100 mg riboflavin per liter of the cell culture.

The term “riboflavin” as used herein refers to the compound 7,8-dimethyl-10-(D-1′-ribityl-) isoalloxazine, as well as derivatives thereof. The term “derivative” refers to any chemically modified form of 7,8-dimethyl-10-(D-1′-ribityl-)isoalloxazine. Such derivatives may, for example, be esters, ethers, acids, lipids, glycosylated forms or salt forms. These derivatives may be provided by the Eremothecium organisms themselves, e.g. in additional biochemical reactions, or may be performed in the culture medium, e.g. by reactants present in said medium. In specific embodiments, the riboflavin may be provided in a crystalline form. Such riboflavin crystals may typically be accumulated in cells.

The determination of the riboflavin content of the Eremothecium cells (or of any other microbiological cells, e.g. control cells of other origin) as well as the determination of the riboflavin content in the culture medium may be carried out by any suitable method known to the person skilled in the art.

The determination of the riboflavin content in a cell culture (and thus also the indication of the amount or accumulation of riboflavin in mg per liter culture (including the amount of riboflavin the cells) as mentioned herein above or below) are in a preferred determination approach based on a cultivation procedure and a subsequent testing procedure, which include the following steps: Typically a 10 ml of pre-culture medium (55 g Yeast extract 50, 0.5 g MgSO₄, pH7.0 with NaOH and filled with 950 ml H₂O; 9.5 ml of this medium+0.5 ml rapeseed oil) is filled in 100 mL Erlenmeyer flasks without baffles. The flasks are typically inoculated with E. gossypii mycelium (1 cm²) grown for 3-4 days on SP medium plates. The flasks are subsequently incubated for about 40 h at about 30° C. and 200 rpm. Subsequently, 1 ml of the pre-culture is used to inoculate about 25 ml of a main culture medium (30 g Yeast extract 50, 20 g Soybean flour, 10 g Glycine, 7 g Sodium glutamate, 2 g KH₂PO₄, 0.5 g MgSO₄, 1.1 g DL-methionine, 0.2 g Inositol, 2.1 g sodium formate, pH7.0 with NaOH and filled with 965 ml with H₂O; 21.2 ml main culture medium+2.8 ml rapeseed oil+0.83 ml Urea solution) filled in 250 mL Erlenmeyer flasks with flat baffles. All flasks are typically weighed to determine the mass before incubation. The cultures are typically incubated for about 6 days at about 30° C. and 200 rpm. After the incubation the flasks are typically weighed again to determine the mass after incubation and therefore to be able to include the evaporation effect during incubation. The approach may be carried out in multiple parallel sequences, preferably with 5 or more, more preferably with 10 or more parallel cultures or clones. Measurements and further cultivation may preferably be performed in duplicates or at least in triplicates to account for statistical differences in the cultures.

Subsequently, the riboflavin content of the entire production culture, i.e. including the riboflavin content of the cells (also including any crystalline form of riboflavin) and the riboflavin excreted from the cells and present in the culture medium may be determined by suitable photometric assays. In a preferred determination approach a photometric assay may be employed which is based on a reaction of the culture medium as obtained according to the above described procedure (or according to any other culturing procedure) with a nicotinamide solution. Preferably, 250 μL of the culture are mixed with about 4.75 mL of a 40% solution of nicotinamide. Subsequently, the mixture may be incubated, e.g. for about 30 to 60 min, preferably for 40 min, at an elevated temperature, e.g. at around 60 to 80° C., preferably at about 70° C. The incubation should preferably be carried out in darkness. Subsequently, samples may be cooled, e.g. for about 5 min, and mixed with water, e.g. with 3 ml of water. The photometric determination of the extinction may be performed at a wavelength of 440 or 450 nm. Particularly preferred is a methodology as described in the Examples, e.g. in Example 5 herein below.

In a further embodiment, the riboflavin determination may be performed via HPLC, e.g. as described in Schmidt et al., Microbiology, 1996, 142, 419-426.

The present invention also envisages alternatives and variants of this approach, as well as riboflavin determination methods which differ from the above disclosed methodology. Such further alternatives would be known to the skilled person and can be derived from suitable textbooks or literature sources.

The term “genetically modifying the Eremothecium organism” or “genetically modified organism of the genus Eromethecium” as used herein means that an Eremothecium organism is altered by any suitable genetic means and methods known to the skilled person in order to produce riboflavin, in particular in order to increase the production of riboflavin. Similarly the term “Eremothecium organism which is genetically modified” as used herein means that an Eremothecium organism has been modified or altered by any suitable genetic means and methods known to the skilled person such that it synthesizes and accumulates riboflavin, in particular such that it increases the synthesis and accumulation of riboflavin. In the present invention the Eremothecium organism is genetically modified to increase the activity of one or more proteins linked to the fatty acid uptake and/or beta-oxidation pathway of said organism.

Methods for genetically modifying organisms belonging to the genus Eremothecium are known to the person skilled in the art and are described in the literature. They comprise commonly used methods for introducing genetic elements or material into Eremothecium so as to be contained in the Eremothecium cells, integrated into the chromosome or extrachromosomally (see, e.g., Jimenez et al., 2005, Applied and Environmental Microbiology 71, 5743-5751), or the removal or destruction, or modification, of genetic elements or sequences present in the genome of Eremothecium (see, e.g. Wendland et al., 2000, Gene 242, 381-391; and Mateos et al., 2006, Applied and Environmental Microbiology 72, 5052-5060).

The term “genetic element” as used herein means any molecular unit which is able to transport genetic information. It accordingly relates to a gene, preferably to a native gene, a chimeric gene, a foreign gene, a transgene or a codon-optimized gene. The term “gene” refers to a nucleic acid molecule or fragment that expresses a specific protein, preferably it refers to nucleic acid molecules including regulatory sequences preceding (5′ non-coding sequences) and following (3′ non-coding sequences) the coding sequence. The term “native gene” refers to a gene as found in nature, e.g. in a wildtype strain of Eremothecium, with its own regulatory sequences. The term “chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. According to the present invention a “foreign gene” refers to a gene not normally found in the Eremothecium host organism, but that is introduced into the Eremothecium host organism by gene transfer. Foreign genes can comprise native genes inserted into a non-native organism, or chimeric genes. The term “transgene” refers to a gene that has been introduced into the genome by a transformation procedure.

A “codon-optimized gene” is a gene having its frequency of codon usage designed to mimic the frequency of preferred codon usage of the host cell, preferably the codon usage has been adapted to the codon usage of an organism belonging to the genus Eremothecium, more preferably to the codon usage of Eremothecium gossypii. In specific embodiments of the present invention the codon usage may also be modified in order to establish a deviation of the primary (nucleotide) coding sequence of a certain gene from the wildtype sequence present in Eremothecium, while keeping the secondary (amino acid) sequence identical or almost identical. The modification of the codon usage in these embodiments may be carried out in order to increase the expression of the gene. In addition, or alternatively, the modification of the codon usage may further be used to maximize the difference on the nucleotide sequence level, i.e. in order to provide the least similar sequence on the nucleotide level, while keeping the amino acid sequence identical or almost identical. The term “almost identical” means that amino acid exchanges may be present which have no or only marginal effect with respect to the enzymatic or biological function of the encoded protein. Such effects can be tested with suitable methods known to the skilled person.

The term “coding sequence” refers to a DNA sequence which codes for a specific amino acid sequence. The term “regulatory sequence” refer to a nucleotide sequence located upstream (5′ non-coding sequences), within, or downstream (3′ non-coding sequences) of a coding sequence, and which influences the transcription, RNA processing or stability, or translation of the associated coding sequence. Regulatory sequences may include promoters, enhancers, translation leader sequences, introns, polyadenylation recognition sequences, RNA processing sites, effector binding sites and stem-loop structures.

The term “promoter” refers to a DNA sequence capable of controlling the expression of a coding sequence or functional RNA. Typically, a coding sequence is located 3′ to a promoter sequence. Promoters may be derived in their entirety from a native gene, or be composed of different elements derived from different promoters found in nature, or even comprise synthetic DNA segments. It is understood by a person skilled in the art that different promoters may direct the expression of a gene at different stages of development, or in response to different environmental or physiological conditions. Promoters that cause a gene to be expressed in most cell types at most times are commonly referred to as constitutive promoters. Typically, since the exact boundaries of regulatory sequences have not been completely defined, DNA fragments of different lengths may have identical promoter activity. On the other hand, promoters that cause a gene to be expressed in specific contexts only, e.g. based on the presence of specific factors, growth stages, temperatures, pH or the presence of specific metabolites etc. are understood as regulable promoters.

The term “3′ non-coding sequences” refers to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, i.e. the presence of RNA transcripts, the RNA•processing or stability, or translation of the associated coding sequence. The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. The term “mRNA” refers to messenger RNA, i.e. RNA that is without introns and•that can be translated into protein by the cell.

The term “operably linked” refers to the association of nucleic acid sequences on a single nucleic acid fragment so that the function of one is affected by the other. In the context of a promoter the term means that a coding sequence is rendered capable of affecting the expression of that coding sequence, i.e., the coding sequence is under the transcriptional control of the promoter. Regulatory elements for driving expression of genes in organisms of the genus Eremothecium are known to the person skilled in the art and are widely described in the literature (see, for example, Jimenez et al., 2005, Appl Environ Microbol, 71, 5743-5751 or Maeting et al., 1999, FEBS letters, 444: 15-21). In a preferred embodiment, coding sequence is operably linked to a GPD promoter.

Within a central embodiment of the present invention, the genetic modification of the Eremothecium organism is linked to the fatty acid uptake of Eremothecium.

The “fatty acid uptake” as used herein refers to a transport process, which allows to bring fatty acids, in particular long-chain fatty acids, across the cell membrane into the Eremothecium cell. This process is typically a multifaceted process which involves several activities. Generally the fatty acid transport process is considered to be subdivided into several steps, including the fatty acid delivery to the membrane, fatty acid translocation across the membrane, fatty acid abstraction and removal of the fatty acids from the membrane. In yeasts fatty acid transport typically requires at least the activities Fat1p, Faa1p and Faa4p. The process of fatty acid transport is apparently driven by the esterifaction of fatty acids as a result of either Faa1p or Faa4p. It is assumed that inter alia Fat1p and Faa1p show functional association and thereby mediate the regulated transport of exogenous long-chain fatty acids.

The fatty acid uptake of Eremothecium organisms appears to be highly similar to the fatty acid uptake of Saccharomyces cerevisiae. In E. gossypii, the AGOS_ACL174W gene (protein form AGOS_ACL174Wp) was identified which is the syntenic homolog of the S. cerevisiae Fat1 gene. Fat1p is a bifunctional protein, which plays central roles in fatty acid trafficking at the level of long-chain fatty acid transport and very long-chain fatty acid activation. Yeast strains containing a deletion in the structural gene for Fat1p are distinct from the wild type cells on the basis of a number of growth and biochemical phenotypes. These strains 1) are compromised in their ability to grow on media containing the fatty acid synthesis inhibitor cerulenin and long-chain fatty acids; 2) show reduced uptake of radioactively labeled long chain fatty acids (see also Zou et al., 2002, Journal Biological Chemistry, 277, 31062-31071). Further, in E. gossypii the AGOS_ABL018C (protein form AGOS_ABL018Cp) was identified which is a syntenic homolog of the S. cerevisiae Faa1 and Faa4 genes.

The term “linking the genetic modification to the fatty acid uptake” as used herein thus relates to a genetic modification which influences the function and/or amount of genes or gene products involved in the fatty acid uptake in Eremothecium as defined herein above. Preferably, the term means that the function of genes or gene products involved in the fatty acid uptake in Eremothecium as defined herein above may be improved and/or that the amount of gene expression or of expressed gene product or activity (expressed protein) may be increased, e.g. by over-expression of the a gene involved in the fatty acid uptake in Eremothecium as defined herein above. Preferably, at least the AGOS_ACL174Wp activity, and optionally also the AGOS_ABL018Cp activity may be increased, e.g. its amount be raised. In further embodiments, the AGOS_ACL174Wp activity and the AGOS_ABL018Cp activity are increased, e.g. their amount is raised.

In another central embodiment of the present invention, the genetic modification of the Eremothecium organism is linked to the beta-oxidation pathway of Eremothecium.

The “beta-oxidation pathway” as used herein refers to a biochemical process by which fatty acid molecules are broken down to generate acetyl-coA, which enters the citric acid cycle. Beta-oxidation pathways differ from organism class to organism class. Mammalian beta-oxidation, for example, relies on peroxisomal and mitochondrial activities, whereas several fungal systems only show peroxisomal beta-oxidation.

The beta-oxidation pathway of Eremothecium organisms appears to be highly similar to the beta-oxidation of Saccharomyces cerevisiae (see also Vorapreeda et al., 2012, Microbiology, 158, 217-228), which is confined to peroxisomes (see also Hiltunen et al., 2003, FEMS Microbiology Reviews, 27, 35-64). Typically, beta-oxidation in peroxisomes comprises core reactions which can be considered as a variation of the tricarboxylic acid (TCA) cycle steps involved in converting succinate to oxaloacetate via a sequence of dehydrogenase, hydratase, and dehydrogenase. The beta-oxidation process in fungi of the Saccharomyces group begins with oxidation of the acyl-CoA substrate to trans-2-enoyl-CoA by FAD enzymes representing acyl-CoA oxidase in peroxisomes. These peroxisomal oxidases, Pox1p/Fox1p in S. cerevisiae, pass electrons directly to oxygen to generate H₂O₂. Acyl-CoA oxidase from S. cerevisiae also accepts short-chain substrates, thereby allowing beta-oxidation to be completed. In fungal systems of the Saccharomyces group, the subsequent hydratase 2 and (3R)-hydroxy-specific dehydrogenase reactions are catalyzed by the activity of Mfe2p/Fox2p, which is a homodimeric multifunctional enzyme. The enzyme has been shown to also hydrate short-chain substrates. At the next reaction of the beta-oxidation cycle the ketoacyl-CoA intermediate undergoes thiolytic cleavage by Pot1p/Fox3p, which represents 3-ketoacyl-CoA thiolase. The products of this last step are acetyl-CoA and a 02-shortened acyl-CoA, the latter acting as substrate for Pox1p/Fox1p. The process may continue until all carbons in the fatty acid are turned into acetyl CoA.

For Eremothecium organisms biochemical activities have been described, which are similar to the S. cerevisiae activities. A peroxisomal oxidase activity is provided by Pox1 analog AGOS_AER358C (protein form AGOS_AER358Cp). A hydratase and dehydrogenase activity similar to the homodimeric multifunctional enzyme Mfe2p/Fox2p is provided by AGOS_AGL060W (protein form AGOS_AGL060Wp). A 3-ketoacyl-CoA thiolase activity similar to Pot1p/Fox3p is provided by AGOS_AFR302W (protein form AGOS_AFR302Wp). Additional activities, which are involved in the beta-oxidation of Eremothecium organisms, in particular of E. gossypii include acyl-CoA-dehydrogenase AGOS_AFL213W (protein form AGOS_AFL213Wp) and acetyl-CoA acteyltransferase AGOS_ADR1650 (protein form AGOS_ADR165Cp).

Additional activities may be required for an efficient performance of beta-oxidation in peroxisomes. These activities include AGOS_AFR453W (protein form AGOS_AFR453Wp), which corresponds to the Pex5 activity of S. cerevisiae, i.e. a receptor for specific types of peroxisomal targeting signals (PTS). Further included is AGOS_ACR128C (protein form AGOS_ACR128Cp), which is a homolog of S. cerevisiae Pxa1, i.e. a peroxisomal fatty acid transport protein, and AGOS_AER091W (protein form AGOS_AER091 Wp), which is a homolog of S. cerevisiae Pxa2, i.e. a further peroxisomal fatty acid transport protein.

The term “linking the genetic modification to the beta-oxidation pathway” as used herein thus relates to a genetic modification which influences the function and/or amount of genes or gene products involved in the beta-oxidation pathway in Eremothecium as defined herein above. Preferably, the term means that the function of genes or gene products involved in the beta-oxidation pathway in Eremothecium as defined herein above may be improved and/or that the amount of gene expression or of expressed gene product or activity (expressed protein) may be increased, e.g. by over-expression of a gene involved in the beta-oxidation pathway in Eremothecium as defined herein above. Preferably, at least one of the activities of AGOS_AER358Cp (Pox1), AGOS_AGL060Wp (Fox2), and AGOS_AFR302Wp (Pot1/Fox3) may be increased, e.g. its amount be raised.

In preferred embodiments, the activity of AGOS_ACL174Wp (Fat1) is provided by a polypeptide comprising, essentially consisting of or consisting of the amino acid sequence of SEQ ID NO: 1 or functional parts or fragments thereof, or is encoded by a nucleic acid comprising, essentially consisting of or consisting of the nucleotide sequence of SEQ ID NO: 2 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 1 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 2 or functional parts or fragments thereof.

In preferred embodiments, the activity of AGOS_ABL018Cp (Faa1/Faa4) is provided by a polypeptide comprising, essentially consisting of or consisting of the amino acid sequence of SEQ ID NO: 3 or functional parts or fragments thereof, or is encoded by a nucleic acid comprising, essentially consisting of or consisting of the nucleotide sequence of SEQ ID NO: 4 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 3 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 4 or functional parts or fragments thereof.

In preferred embodiments, the activity of AGOS_AER358Cp (Pox1) is provided by a polypeptide comprising, essentially consisting of or consisting of the amino acid sequence of SEQ ID NO: 5 or functional parts or fragments thereof, or is encoded by a nucleic acid comprising, essentially consisting of or consisting of the nucleotide sequence of SEQ ID NO: 6 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 5 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 6 or functional parts or fragments thereof.

In further preferred embodiments, the activity of AGOS_AGL060Wp (Fox2) is provided by a polypeptide comprising, essentially consisting of or consisting of the amino acid sequence of SEQ ID NO: 7 or functional parts or fragments thereof, or is encoded by a nucleic acid comprising, essentially consisting of or consisting of the nucleotide sequence of SEQ ID NO: 8 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 7 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 8 or functional parts or fragments thereof.

In further preferred embodiments, the activity of AGOS_AFR302Wp (Pot1/Fox3) is provided by a polypeptide comprising, essentially consisting of or consisting of the amino acid sequence of SEQ ID NO: 9 or functional parts or fragments thereof, or is encoded by a nucleic acid comprising, essentially consisting of or consisting of the nucleotide sequence of SEQ ID NO: 10 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 9 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 10 or functional parts or fragments thereof.

All sequences disclosed herein have been obtained from Eremothecium gossypii strain ATCC 10895.

The functional fragment or functional part of the amino acid sequence of SEQ ID No. 1 has a length of at least 300 or 350 amino acids, preferably of at least 400 or 450 amino acids, more preferably of at least 500 or 550 amino acids and most preferably of at least 600 or 620 amino acids.

The functional fragment or functional part of the amino acid sequence of SEQ ID No. 3 has a length of at least 300 or 350 amino acids, preferably of at least 400 or 450 amino acids, more preferably of at least 500 or 550 amino acids and most preferably of at least 580 or 600 amino acids.

The functional fragment or functional part of the amino acid sequence of SEQ ID No. 5 has a length of at least 400 or 450 amino acids, preferably of at least 500 or 550 amino acids, more preferably of at least 600 or 650 amino acids and most preferably of at least 700 or 720 amino acids.

The functional fragment or functional part of the amino acid sequence of SEQ ID No. 7 has a length of at least 450 or 500 amino acids, preferably of at least 550 or 600 amino acids, more preferably of at least 650 or 700 amino acids and most preferably of at least 750, 800 or 850 amino acids.

The functional fragment or functional part of the amino acid sequence of SEQ ID No. 9 has a length of at least 150 or 200 amino acids, preferably of at least 250 or 300 amino acids, more preferably of at least 320 or 340 amino acids and most preferably of at least 360 or 380 amino acids.

A “functional fragment” or “functional part” has essentially the same activity as the full-length protein, i.e. it has an activity which is at least 50%, 55%, 60% or 65%, preferably at least 70%, 75% or 80%, more preferably at least 85%, 90% or 95% and most preferably 100% of the activity of the full-length protein.

Within the meaning of the present invention, “sequence identity” denotes the degree of conformity with regard to the 5′-3′ sequence within a nucleic acid molecule in comparison to another nucleic acid molecule. The sequence identity may be determined using a series of programs, which are based on various algorithms, such as BLASTN, ScanProsite, the laser gene software, etc. As an alternative, the BLAST program package of the National Center for Biotechnology Information (http://www.ncbi.nlm.nih.gov/) may be used with the default parameters. In addition, the program Sequencher (Gene Codes Corp., Ann Arbor, Mich., USA) using the “dirtydata”-algorithm for sequence comparisons may be employed.

The sequence identity refers to the degree of sequence identity over a length of 150, 200 or 250 amino acids, preferably 300, 350, 400, 450 or 500 amino acids, more preferably 550 or 600, amino acids and most preferably the whole length of the amino acid sequence according to SEQ ID No. 1

The sequence identity refers to the degree of sequence identity over a length of 500, 600 or 700 nucleotides, preferably 800, 900, 1000, 1100 or 1200 nucleotides, more preferably 1300, 1400, 1500, 1600, 1700 or 1800 nucleotides and most preferably the whole length of the nucleic acid sequence according to SEQ ID No. 2.

The sequence identity refers to the degree of sequence identity over a length of 150, 200 or 250 amino acids, preferably 300, 350, 400, 450 or 500 amino acids, more preferably 550 or 600 amino acids and most preferably the whole length of the amino acid sequence according to SEQ ID No. 3.

The sequence identity refers to the degree of sequence identity over a length of 500, 600 or 700 nucleotides, preferably 800, 900, 1000, 1100 or 1200 nucleotides, more preferably 1300, 1400, 1500, 1600, 1700, 1800 or 1900 nucleotides and most preferably the whole length of the nucleic acid sequence according to SEQ ID No. 4.

The sequence identity refers to the degree of sequence identity over a length of 250, 300 or 350 amino acids, preferably 400, 450, 500, 550 or 600 amino acids, more preferably 650 or 700 amino acids and most preferably the whole length of the amino acid sequence according to SEQ ID No. 5.

The sequence identity refers to the degree of sequence identity over a length of 600, 700 or 800 nucleotides, preferably 900, 1000, 1100, 1200 or 1300 nucleotides, more preferably 1400, 1500, 1600, 1700, 1800, 1900, 2000 or 2100 nucleotides and most preferably the whole length of the nucleic acid sequence according to SEQ ID No. 6.

The sequence identity refers to the degree of sequence identity over a length of 350, 400 or 450 amino acids, preferably 500, 550, 600, 650 or 700 amino acids, more preferably 750, 800 or 850 amino acids and most preferably the whole length of the amino acid sequence according to SEQ ID No. 7.

The sequence identity refers to the degree of sequence identity over a length of 800, 900 or 1000 nucleotides, preferably 1100, 1200, 1300, 1400, 1500, 1600 or 1700 nucleotides, more preferably 1800, 1900, 2000, 2100, 2200, 2300, 2400 or 2500 nucleotides and most preferably the whole length of the nucleic acid sequence according to SEQ ID No. 8.

The sequence identity refers to the degree of sequence identity over a length of 150, 180 or 200 amino acids, preferably 220, 240, 260, 280 or 300 amino acids, more preferably 320, 340, 360 or 380 amino acids and most preferably the whole length of the amino acid sequence according to SEQ ID No. 9.

The sequence identity refers to the degree of sequence identity over a length of 400, 500 or 550 nucleotides, preferably 600, 650, 700 or 750 nucleotides, more preferably 800, 850, 900, 950, 1000, 1050, 1100 or 1150 nucleotides and most preferably the whole length of the nucleic acid sequence according to SEQ ID No. 10.

The polypeptide having an amino acid sequence with at least 70% sequence identity to the sequences according to any of SEQ ID Nos. 1, 3, 5, 7, and 9 has essentially the same activity as the protein according to any of SEQ ID Nos. 1, 3, 5, 7, and 9, i.e. it has an activity which is at least 50%, 55%, 60% or 65%, preferably at least 70%, 75% or 80%, more preferably at least 85%, 90% or 95% and most preferably 100% of the activity of the protein according to any of SEQ ID Nos. 1, 3, 5, 7, and 9.

The nucleic acid sequence with at least 70% sequence identity to the nucleic acid sequence according to any of SEQ ID Nos. 2, 4, 6, 8, and 10 encodes a protein having essentially the same activity as the protein encoded by a nucleic acid sequence according to any of SEQ ID Nos. 2, 4, 6, 8, and 10, i.e. it has an activity which is at least 50%, 55%, 60% or 65%, preferably at least 70%, 75% or 80%, more preferably at least 85%, 90% or 95% and most preferably 100% of the activity of the protein encoded by a nucleic acid sequence according to any of SEQ ID Nos. 2, 4, 6, 8, and 10.

Additionally or alternatively, at least one of further activities of the beta-oxidation pathway in Eremothecium such as AGOS_AFL213Wp, AGOS_ADR165Cp, AGOS_AFR453Wp (Pex5), AGOS_ACR128Cp (Pxa1) or AGOS_AER091Wp (Pxa2) may be modified, e.g. increased, in the context of the present invention.

In preferred embodiments, the activity of AGOS_AFL213Wp is provided by a polypeptide comprising, essentially consisting of or consisting of the amino acid sequence of SEQ ID NO: 11 or functional parts or fragments thereof, or is encoded by a nucleic acid comprising, essentially consisting of or consisting of the nucleotide sequence of SEQ ID NO: 12 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 11 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 12 or functional parts or fragments thereof.

In further preferred embodiments, the activity of AGOS_ADR165Cp is provided by a polypeptide comprising, essentially consisting of or consisting of the amino acid sequence of SEQ ID NO: 13 or functional parts or fragments thereof, or is encoded by a nucleic acid comprising, essentially consisting of or consisting of the nucleotide sequence of SEQ ID NO: 14 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 13 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 14 or functional parts or fragments thereof.

In further preferred embodiments, the activity of AGOS_AFR453Wp (Pex5) is provided by a polypeptide comprising, essentially consisting of or consisting of the amino acid sequence of SEQ ID NO: 15 or functional parts or fragments thereof, or is encoded by a nucleic acid comprising, essentially consisting of or consisting of the nucleotide sequence of SEQ ID NO: 16 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 15 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 16 or functional parts or fragments thereof.

In further preferred embodiments, the activity of AGOS_ACR128Cp (Pxa1) is provided by a polypeptide comprising, essentially consisting of or consisting of the amino acid sequence of SEQ ID NO: 17 or functional parts or fragments thereof, or is encoded by a nucleic acid comprising, essentially consisting of or consisting of the nucleotide sequence of SEQ ID NO: 18 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 17 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 18 or functional parts or fragments thereof.

In further preferred embodiments, the activity of AGOS_AER091Wp (Pxa2) is provided by a polypeptide comprising, essentially consisting of or consisting of the amino acid sequence of SEQ ID NO: 19 or functional parts or fragments thereof, or is encoded by a nucleic acid comprising, essentially consisting of or consisting of the nucleotide sequence of SEQ ID NO: 20 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 19 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 20 or functional parts or fragments thereof.

The term “linking the genetic modification to the beta-oxidation pathway” as used herein thus relates to a genetic modification which influences the function and/or amount of genes or gene products involved in the beta-oxidation pathway in Eremothecium as defined herein above. Preferably, the term means that the function of genes or gene products involved in the beta oxidation pathway in Eremothecium as defined herein above may be improved and/or that the amount of gene expression or of expressed gene product or activity (expressed protein) may be increased, e.g. by over-expression of the a gene involved in the beta oxidation pathway in Eremothecium as defined herein above. Preferably, at least the AGOS_AER358Cp (Pox1) activity, or the AGOS_AGL060Wp (Fox2), or the AGOS_AFR302Wp (Pot1/Fox3) may be increased, or its amount be raised. In further preferred embodiments, the AGOS_AGL060Wp (Fox2) and the AGOS_AFR302Wp (Pot1/Fox3) may be increased or their amount be raised. In yet a further preferred embodiment the AGOS_AER358Cp (Pox1) activity and the AGOS_AGL060Wp (Fox2) activity and the AGOS_AFR302Wp (Pot1/Fox3) activity may be increased or their amount be raised.

In further embodiments, a genetic modification to the beta-oxidation pathway as described above, may relate to a genetic modification which influences the function and/or amount of further genes or gene products involved in the beta-oxidation pathway. Such additional genes or gene products may be AGOS_AFL213Wp, AGOS_ADR165Cp, AGOS_AFR453Wp (Pex5), AGOS_ACR128Cp (Pxa1) and/or AGOS_AER091Wp (Pxa2). Particularly preferred are genetic modifications, in which the AGOS_AER358Cp (Pox1) activity and/or the AGOS_AGL060Wp (Fox2) activity and/or the AGOS_AFR302Wp (Pot1/Fox3) activity may be increased or their amount be raised and wherein additionally one or more of the AGOS_AFL213Wp, AGOS_ADR165Cp, AGOS_AFR453Wp (Pex5), AGOS_ACR128Cp (Pxa1) or AGOS_AER091Wp (Pxa2) activity are increased, or their amount be raised.

In further preferred embodiments, a genetic modification may be linked to the fatty acid uptake and a further genetic modification may be linked to the beta-oxidation pathway as described above. A correspondingly modified organism may thus comprise a genetic modification may be linked to the fatty acid uptake and at the same time a genetic modification linked to the beta-oxidation pathway. Preferably, the function of genes or gene products involved in the beta oxidation pathway in Eremothecium as defined herein above and the function of genes involved in fatty acid uptake may be improved and/or that the amount of gene expression or of expressed gene product or activity (expressed protein) may be increased, e.g. by over-expression of the a gene involved in the beta oxidation pathway in Eremothecium as defined herein above and a gene involved in fatty acid uptake in Eremothecium as defined herein above. For example, the AGOS_ACL174Wp (Fat1) activity and (i) the AGOS_AER358Cp (Pox1) activity, or (ii) the AGOS_AGL060Wp (Fox2), or (iii) the AGOS_AFR302Wp (Pot1/Fox3) may be increased.

Alternatively the AGOS_ACL174Wp (Fat1) activity and (i) the AGOS_AER358Cp (Pox1) activity, and (ii) the AGOS_AGL060Wp (Fox2) may be increased. In a further alternative, the AGOS_ACL174Wp (Fat1) activity and (i) the AGOS_AER358Cp (Pox1) activity and (iii) the AGOS_AFR302Wp (Pot1/Fox3) may be increased. In yet another alternative, the AGOS_ACL174Wp (Fat1) activity and (ii) the AGOS_AGL060Wp (Fox2), and (iii) the AGOS_AFR302Wp (Pot1/Fox3) may be increased. In further embodiments, the AGOS_ACL174Wp (Fat1) activity and (ii) the AGOS_AGL060Wp (Fox2) and (iii) the AGOS_AFR302Wp (Pot1/Fox3) may be increased.

In yet another type of embodiments, the AGOS_ABL018Cp (Faa1/Faa4) activity and (i) the AGOS_AER358Cp (Pox1) activity, or (ii) the AGOS_AGL060Wp (Fox2), or (iii) the AGOS_AFR302Wp (Pot1/Fox3) may be increased.

Alternatively the AGOS_ABL018Cp (Faa1/Faa4) activity and (i) the AGOS_AER358Cp (Pox1) activity, and (ii) the AGOS_AGL060Wp (Fox2) may be increased. In a further alternative, the AGOS_ABL018Cp (Faa1/Faa4) activity and (i) the AGOS_AER358Cp (Pox1) activity and (ii) the AGOS_AFR302Wp (Pot1/Fox3) may be increased. In yet another alternative, the AGOS_ABL018Cp (Faa1/Faa4) activity and (i) the AGOS_AGL060Wp (Fox2), and (ii) the AGOS_AFR302Wp (Pot1/Fox3) may be increased. In further embodiments, the AGOS_ABL018Cp (Faa1/Faa4) activity and (i) the AGOS_AGL060Wp (Fox2) and (ii) the AGOS_AFR302Wp (Pot1/Fox3) may be increased.

In yet another type of embodiments, the AGOS_ACL174Wp (Fat1) activity and the AGOS_ABL018Cp (Faa1/Faa4) activity and (i) the AGOS_AER358Cp (Pox1) activity, or (ii) the AGOS_AGL060Wp (Fox2), or (iii) the AGOS_AFR302Wp (Pot1/Fox3) may be increased.

Alternatively the AGOS_ACL174Wp (Fat1) activity and the AGOS_ABL018Cp (Faa1/Faa4) activity and (i) the AGOS_AER358Cp (Pox1) activity, and (ii) the AGOS_AGL060Wp (Fox2) may be increased. In a further alternative, the AGOS_ACL174Wp (Fat1) activity and the AGOS_ABL018Cp (Faa1/Faa4) activity and (i) the AGOS_AER358Cp (Pox1) activity and (ii) the AGOS_AFR302Wp (Pot1/Fox3) may be increased. In yet another alternative, the AGOS_ACL174Wp (Fat1) activity and the AGOS_ABL018Cp (Faa1/Faa4) activity and (i) the AGOS_AGL060Wp (Fox2), and (ii) the AGOS_AFR302Wp (Pot1/Fox3) may be increased. In further embodiments, the AGOS_ACL174Wp (Fat1) activity and (i) the AGOS_ABL018Cp (Faa1/Faa4) activity and (ii) the AGOS_AGL060Wp (Fox2) and (iii) the AGOS_AFR302Wp (Pot1/Fox3) may be increased.

The increase of the activity of AGOS_ACL174Wp (Fat1) may be due to an over-expression of the AGOS_ACL174W (fat1) gene. An increase of the activity of AGOS_ABL018Cp (Faa1/Faa4) may be due to an over-expression of the AGOS_ABL0180 (faa1/faa4) gene. An increase of the activity of AGOS_AER358Cp (Pox1) may be due to an over-expression of the AGOS_AER358C (pox1) gene. An increase of the activity of AGOS_AGL060Wp (Fox2) may be due to an over-expression of the AGOS_AGL060W (fox2) gene. An increase of the activity of AGOS_AFR302Wp (Pot1/Fox3) may be due to an over-expression of the AGOS_AFR302W (pot1/fox3) gene. Further specifically envisaged is the co-over-expression of the AGOS_AGL060W (fox2) gene and the AGOS_AFR302W (pot1/fox3) gene in order to increase the activity of AGOS_AGL060Wp (Fox2) and AGOS_AFR302Wp (Pot1/Fox3). In preferred embodiments, the Fat1, Faa1/Faa4, Pox1, Fox2 or Pot1/Fox3 activities are provided by the specific polypeptides and/or encoded by the specific nucleic acids as defined herein above. In further preferred embodiments the fat 1 gene, the faa1/faa4 gene, the pox1 gene, fox 2 gene or fox3 gene correspond to, comprise, essentially consist of or consist of the sequences of SEQ ID NO: 2, 4, 6, 8, or 10, respectively, or homologous sequences thereof as defined herein above.

Further envisaged are multiple over-expression events of any of the above genes. For example, AGOS_AER358C (pox1) and AGOS_AGL060W (fox2) may be over-expressed, or AGOS_AER358C (pox1) and AGOS_AFR302W (pot1/fox3) may be over-expressed, or AGOS_AGL060W (fox2) and AGOS_AFR302W (pot1/fox3) may be over-expressed, or AGOS_AER358C (pox1) and any of AGOS_AFL213W, AGOS_ADR1650, AGOS_AFR453W (pex5), AGOS_ACR128C (pxa1) or AGOS_AER091W (pxa2) may be over-expressed, or AGOS_AGL060W (fox2) and any of AGOS_AFL213W, AGOS_ADR1650, AGOS_AFR453W (pex5), AGOS_ACR128C (pxa1) may be over-expressed, or AGOS_AER091W (pxa2), or AGOS_AFR302W (pot1/fox3) and any of AGOS_AFL213W, AGOS_ADR1650, AGOS_AFR453W (pex5), AGOS_ACR128C (pxa1) may be over-expressed.

In further examples, AGOS_ACL174W (fat1) and AGOS_AER358C (pox1) and AGOS_AGL060W (fox2) may be over-expressed, or AGOS_ACL174W (fat1) and AGOS_AER358C (pox1) and AGOS_AFR302W (pot1/fox3) may be over-expressed, or AGOS_ACL174W (fat1) and AGOS_AGL060W (fox2) and AGOS_AFR302W (pot1/fox3) may be over-expressed, or AGOS_ACL174W (fat1) and AGOS_AER358C (pox1) and any of AGOS_AFL213W, AGOS_ADR1650, AGOS_AFR453W (pex5), AGOS_ACR128C (pxa1) or AGOS_AER091W (pxa2) may be over-expressed, or AGOS_ACL174W (fat1) and AGOS_AGL060W (fox2) and any of AGOS_AFL213W, AGOS_ADR1650, AGOS_AFR453W (pex5), AGOS_ACR128C (pxa1) may be over-expressed, or AGOS_ACL174W (fat1) and AGOS_AER091W (pxa2), or AGOS_ACL174W (fat1) and AGOS_AFR302W (pot1/fox3) and any of AGOS_AFL213W, AGOS_ADR1650, AGOS_AFR453W (pex5), AGOS_ACR128C (pxa1) may be over-expressed.

In further examples, AGOS_ABL018C (faa1/faa4) and AGOS_AER358C (pox1) and AGOS_AGL060W (fox2) may be over-expressed, or AGOS_ABL018C (faa1/faa4) and AGOS_AER358C (pox1) and AGOS_AFR302W (pot1/fox3) may be over-expressed, or AGOS_ABL018C (faa1/faa4) and AGOS_AGL060W (fox2) and AGOS_AFR302W (pot1/fox3) may be over-expressed, or AGOS_ABL018C (faa1/faa4) and AGOS_AER358C (pox1) and any of AGOS_AFL213W, AGOS_ADR1650, AGOS_AFR453W (pex5), AGOS_ACR128C (pxa1) or AGOS_AER091W (pxa2) may be over-expressed, or AGOS_ABL018C (faa1/faa4) and AGOS_AGL060W (fox2) and any of AGOS_AFL213W, AGOS_ADR1650, AGOS_AFR453W (pex5), AGOS_ACR128C (pxa1) may be over-expressed, or AGOS_ABL018C (faa1/faa4) and AGOS_AER091W (pxa2), or AGOS_ABL0180 (faa1/faa4) and AGOS_AFR302W (pot1/fox3) and any of AGOS_AFL213W, AGOS_ADR1650, AGOS_AFR453W (pex5), AGOS_ACR128C (pxa1) may be over-expressed.

In further examples, AGOS_ACL174W (fat1) and AGOS_ABL018C (faa1/faa4) and AGOS_AER358C (pox1) and AGOS_AGL060W (fox2) may be over-expressed, or AGOS_ACL174W (fat1) and AGOS_ABL018C (faa1/faa4) and AGOS_AER358C (pox1) and AGOS_AFR302W (pot1/fox3) may be over-expressed, or AGOS_ACL174W (fat1) and AGOS_ABL018C (faa1/faa4) and AGOS_AGL060W (fox2) and AGOS_AFR302W (pot1/fox3) may be over-expressed, or AGOS_ACL174W (fat1) and AGOS_ABL018C (faa1/faa4) and AGOS_AER358C (pox1) and any of AGOS_AFL213W, AGOS_ADR1650, AGOS_AFR453W (pex5), AGOS_ACR128C (pxa1) or AGOS_AER091W (pxa2) may be over-expressed, or AGOS_ACL174W (fat1) and AGOS_ABL018C (faa1/faa4) and AGOS_AGL060W (fox2) and any of AGOS_AFL213W, AGOS_ADR1650, AGOS_AFR453W (pex5), AGOS_ACR128C (pxa1) may be over-expressed, or AGOS_ACL174W (fat1) and AGOS_ABL018C (faa1/faa4) and AGOS_AER091W (pxa2), or AGOS_ACL174W (fat1) and AGOS_ABL018C (faa1/faa4) and AGOS_AFR302W (pot1/fox3) and any of AGOS_AFL213W, AGOS_ADR1650, AGOS_AFR453W (pex5), AGOS_ACR128C (pxa1) may be over-expressed.

In preferred embodiments, the Fat1, Faa1/Faa4, Pox1, Fox2 or Pot1/Fox3 or the AGOS_AFL213Wp, AGOS_ADR165Cp, AGOS_AFR453Wp (Pex5), AGOS_ACR128Cp (Pxa1) or AGOS_AER091Wp (Pxa2) activities are provided by the specific polypeptides and/or encoded by the specific nucleic acids as defined herein above.

Further envisaged is the over-expression of AGOS_AFL213W and AGOS_ADR1650, or the over-expression of AGOS_AFL213W and AGOS_AFR453W (pex5), or the over-expression of AGOS_AFL213W and AGOS_ACR128C (pxa1), or the over-expression of AGOS_AFL213W and AGOS_AER091W (pxa2); or the over-expression of AGOS_ADR1650 and AGOS_AFR453W (pex5), or the over-expression of AGOS_ADR1650 and AGOS_ACR128C (pxa1), or the over-expression of AGOS_ADR1650 and AGOS_AER091W (pxa2); or the over-expression of AGOS_AFR453W (pex5) and AGOS_ACR128C (pxa1), or the over-expression of AGOS_AFR453W (pex5) and AGOS_AER091W (pxa2); or the over-expression of AGOS_ACR128C (pxa1) and AGOS_AER091W (pxa2). Further envisaged is the over-expression of AGOS_ACL174W (fat1) and AGOS_AFL213W and AGOS_ADR1650, or the over-expression of AGOS_ACL174W (fat1) and AGOS_AFL213W and AGOS_AFR453W (pex5), or the over-expression of AGOS_ACL174W (fat1) and AGOS_AFL213W and AGOS_ACR128C (pxa1), or the over-expression of AGOS_ACL174W (fat1) and AGOS_AFL213W and AGOS_AER091W (pxa2); or the over-expression of AGOS_ACL174W (fat1) and AGOS_ADR1650 and AGOS_AFR453W (pex5), or the over-expression of AGOS_ACL174W (fat1) and AGOS_ADR165C and AGOS_ACR128C (pxa1), or the over-expression of AGOS_ACL174W (fat1) and AGOS_ADR1650 and AGOS_AER091W (pxa2); or the over-expression of AGOS_ACL174W (fat1) and AGOS_AFR453W (pex5) and AGOS_ACR128C (pxa1), or the over-expression of AGOS_ACL174W (Fat1) and AGOS_AFR453W (pex5) and AGOS_AER091W (pxa2); or the over-expression of AGOS_ACL174W (fat1) and AGOS_ACR128C (pxa1) and AGOS_AER091W (pxa2). Also envisaged is the over-expression of AGOS_ACL174W (Fat1) and AGOS_ABL018C (Faa1/Faa4) and AGOS_AFL213W and AGOS_ADR1650, or the over-expression of AGOS_ACL174W (fat1) and AGOS_ABL018C (faa1/faa4) and AGOS_AFL213W and AGOS_AFR453W (pex5), or the over-expression of AGOS_ACL174W (fat1) and AGOS_ABL018C (faa1/faa4) and AGOS_AFL213W and AGOS_ACR128C (pxa1), or the over-expression of AGOS_ACL174W (fat1) and AGOS_ABL0180 (faa1/faa4) and AGOS_AFL213W and AGOS_AER091W (pxa2); or the over-expression of AGOS_ACL174W (fat1) and AGOS_ABL0180 (faa1/faa4) and AGOS_ADR1650 and AGOS_AFR453W (pex5), or the over-expression of AGOS_ACL174W (fat1) and AGOS_ABL018C (faa1/faa4) and AGOS_ADR165C and AGOS_ACR128C (pxa1), or the over-expression of AGOS_ACL174W (fat1) and AGOS_ABL018C (faa1/faa4) and AGOS_ADR1650 and AGOS_AER091W (pxa2); or the over-expression of AGOS_ACL174W (fat1) and AGOS_ABL0180 (faa1/faa4) and AGOS_AFR453W (pex5) and AGOS_ACR128C (pxa1), or the over-expression of AGOS_ACL174W (fat1) and AGOS_ABL018C (faa1/faa4) and AGOS_AFR453W (pex5) and AGOS_AER091W (pxa2); or the over-expression of AGOS_ACL174W (fat1) and AGOS_ABL018C (faa1/faa4) and AGOS_ACR128C (pxa1) and AGOS_AER091W (pxa2). In preferred embodiments, the AGOS_ACL174W (fat1), AGOS_ABL0180 (faa1/faa4), AGOS_AFL213W, AGOS_ADR1650, AGOS_AFR453W (pex5), AGOS_ACR128C (pxa1) or AGOS_AER091W (pxa2) activities are provided by the specific polypeptides and/or encoded by the specific nucleic acids as defined herein above.

Further envisaged are specific over-expression situations, such as the over-expression of AGOS_AER358C (pox1), AGOS_AGL060W (fox2) and AGOS_AFR302W (pot1/fox3), or the over-expression of AGOS_AER358C (pox1) and two activities of AGOS_AFL213W, AGOS_ADR1650, AGOS_AFR453W (pex5), AGOS_ACR128C (pxa1) or AGOS_AER091W (pxa2); or the over-expression of AGOS_AGL060W (fox2) and two of AGOS_AFL213W, AGOS_ADR1650, AGOS_AFR453W (pex5), AGOS_ACR128C (pxa1) or AGOS_AER091W (pxa2); or the over-expression of AGOS_AFR302W (pot1/fox3) and two activities of AGOS_AFL213W, AGOS_ADR1650, AGOS_AFR453W (pex5), AGOS_ACR128C (pxa1) or AGOS_AER091 W (pxa2); or the over-expression of AGOS_ACL174W (fat1) and two of AGOS_AFL213W, AGOS_ADR1650, AGOS_AFR453W (pex5), AGOS_ACR128C (pxa1) or AGOS_AER091W (pxa2); or the over-expression AGOS_ABL018C (faa1/faa4), and two of AGOS_AFL213W, AGOS_ADR1650, AGOS_AFR453W (pex5), AGOS_ACR128C (pxa1) or AGOS_AER091W (pxa2); or the over-expression of AGOS_ACL174W (fat1), AGOS_ABL018Cp (faa1/faa4), and two of AGOS_AFL213W, AGOS_ADR1650, AGOS_AFR453W (pex5), AGOS_ACR128C (pxa1) or AGOS_AER091W (pxa2). Further envisaged are over-expression situations in which AGOS_AER358C (pox1) and AGOS_AGL060W (fox2) and one of AGOS_AFL213W, AGOS_ADR1650, AGOS_AFR453W (pex5), AGOS_ACR128C (pxa1) or AGOS_AER091W (pxa2) are over-expressed; or in which AGOS_AER358C (pox1) and AGOS_AFR302W (pot1/fox3) and one of AGOS_AFL213W, AGOS_ADR1650, AGOS_AFR453W (pex5), AGOS_ACR128C (pxa1) or AGOS_AER091W (pxa2) are over-expressed; or in which AGOS_AGL060W (fox2) and AGOS_AFR302W (pot1/fox3) and one of AGOS_AFL213W, AGOS_ADR1650, AGOS_AFR453W (pex5), AGOS_ACR128C (pxa1) or AGOS_AER091W (pxa2), or in which AGOS_AER358C (pox1) and AGOS_AGL060W (fox2) and AGOS_ACL174W (fat1) and/or AGOS_ABL018C (faa1/faa4) and one of AGOS_AFL213W, AGOS_ADR1650, AGOS_AFR453W (pex5), AGOS_ACR128C (pxa1) or AGOS_AER091W (pxa2) are over-expressed; or in which AGOS_AER358C (pox1) and AGOS_AFR302W (pot1/fox3) and AGOS_ACL174W (fat1) and/or AGOS_ABL018C (faa1/faa4) and one of AGOS_AFL213W, AGOS_ADR1650, AGOS_AFR453W (pex5), AGOS_ACR128C (pxa1) or AGOS_AER091W (pxa2) are over-expressed; or in which AGOS_AGL060W (fox2) and AGOS_AFR302W (pot1/fox3) and AGOS_ACL174W (fat1) and/or AGOS_ABL0180 (faa1/faa4) and one of AGOS_AFL213W, AGOS_ADR1650, AGOS_AFR453W (pex5), AGOS_ACR128C (pxa1) or AGOS_AER091W (pxa2) are over-expressed. Further envisaged are 4, 5, 6, 7 or 8 over-expression situations, in which 4, 5, 6, 7 or 8 genes involved in beta oxidation in Eremothecium as defined herein above and/or 1 or 2 of the genes involved in fatty acid uptake are over-expressed. In preferred embodiments, the Fat1, Faa1/Faa4, Pox1, Fox2 or Pot1/Fox3 or the AGOS_AFL213Wp, AGOS_ADR165Cp, AGOS_AFR453Wp (Pex5), AGOS_ACR128Cp (Pxa1) or AGOS_AER091Wp (Pxa2) activities are provided by the specific polypeptides and/or encoded by the specific nucleic acids as defined herein above.

The term “increase of activity” or “increase of amount” as used herein refers to any modification of the genetic element encoding an enzymatic activity, e.g. on a molecular basis, the transcript expressed by the genetic element or the protein or enzymatic activity encoded by said genetic element, which leads to an increase of said enzymatic activity, an increase of the concentration of said enzymatic activity in the cell and/or an improvement of the functioning of said activity. The activity can be measured with suitable tests or assays, which would be known to the skilled person or can be derived from suitable literature sources such as Small et al., Biochem. J, 1985, 227, 205-210, which discloses an assay for peroxisomal acyl-CoA oxidase activity; Watkins et al., The Journal of Biological Chemistry, 1998, 273(29), 18210-18219, which discloses methods for the measurement of acyl-CoA synthetase activity; Hiltunen et al., The Journal of Biological Chemistry, 1992, 267(10), 6646-6653, which discloses an assay for Fox2 activity; or Lee et al., BMB reports, 2009, 42(5), 281-285, which discloses an assay for Pot1 activity.

A modification of the genetic element encoding an enzymatic activity may, for example, lead to an increase of activity of about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original activity (without modification) in the context of the same organism. In preferred embodiments, such increase of activity may be provided for at least one, or more than one, e.g. 2, 3, 4, 5, 6, or more, or all of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and AGOS_AER091Wp (Pxa2). In preferred embodiments, the activities which are increase are represented by, comprise, essentially consist of, or consist of one, e.g. 2, 3, 4, 5, 6, or more, or all of the amino acid sequences of SEQ ID NO: 1, 3, 5, 7, 9, 11, 13, 15, 17 and/or 19, or homologous sequences thereof as defined herein above.

In specific embodiments, the increase of activity is due to the expression and, in particular the over-expression of the genetic element whose expression yields the activity as mentioned above. The term “expression”, as used herein refers to the transcription and accumulation of sense strand (mRNA) derived from nucleic acid molecules or genes as mentioned herein. More preferably, the term also refers to the translation of mRNA into a polypeptide or protein and the corresponding provision of such polypeptides or proteins within the cell. In typical embodiments, the expression may be an over-expression. The term “over-expression” relates to the accumulation of more transcripts and in particular of more polypeptides or proteins than upon the expression an endogenous copy of the genetic element which gives rise to said polypeptide or protein in the context of the same organism. In further, alternative embodiments, the term may also refer to the accumulation of more transcripts and in particular of more polypeptides or proteins than upon the expression of typical, moderately expressed housekeeping genes such as beta-actin or beta-tubulin.

In a particularly preferred embodiment the increase of the AGOS_ACL174Wp (Fat1) activity is due to the over-expression of the AGOS_ACL174W gene (fat1); and/or the increase of the AGOS_AER358Cp (Pox1) activity is due to the over-expression of the AGOS_AER358C gene (pox1); and/or the increase of the AGOS_ABL018Cp (FAA1/FAA4) activity is due to the over-expression of the AGOS_ABL018C gene (faa1/faa4) and/or the increase of the AGOS_AGL060Wp (Fox2) activity and the AGOS_AFR302Wp (Pot1/Fox3) activity is due to the over-expression of the AGOS_AGL060W gene (fox2) and the AGOS_AFR302W gene (pot1/fox3).

In preferred embodiments, the over-expression as mentioned above may lead to an increase in the transcription rate of a gene of about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original transcription (without modification or over-expression) in the context of the same organism. In preferred embodiments, such increase of in the transcription rate of a gene may be provided for at least one, or more than one, e.g. 2, 3, 4, 5, 6, or more, or all of AGOS_ACL174W (fat1), AGOS_ABL0180 (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) and AGOS_AER091W (pxa2). In preferred embodiments, the transcription rates which are increased refer to one, e.g. 2, 3, 4, 5, 6, or more, or all of the transcripts of the nucleotide sequences of SEQ ID NO: 2, 4, 6, 8, 10, 12, 14, 16, 18 and/or 20, or homologous sequences thereof as defined herein above.

In further preferred embodiments, the over-expression may lead to an increase in the amount of mRNA of a gene of about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original transcription (without modification or over-expression) in the context of the same organism. In preferred embodiments, such increase in the amount of mRNA of a gene may be provided for at least one, or more than one, e.g. 2, 3, 4, 5, 6, or more, or all of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) and AGOS_AER091W (pxa2). In preferred embodiments, the amount of mRNA which is increased is refers to mRNA comprising, essentially consisting of, or consisting of one, e.g. 2, 3, 4, 5, 6, or more, or all of the nucleotide sequences of SEQ ID NO: 2, 4, 6, 8, 10, 12, 14, 16, 18 and/or 20, or homologous sequences thereof as defined herein above.

In yet another preferred embodiment, the over-expression may lead to an increase in the amount of polypeptide or protein encoded by the over-expressed gene of about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% or any value in between these values in comparison to the corresponding wildtype or original amount of polypeptide or protein (without modification or over-expression) in the context of the same organism. In preferred embodiments, such increase in the amount polypeptide or protein encoded by the over-expressed gene may be provided for at least one, or more than one, e.g. 2, 3, 4, 5, 6, or more, or all of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and AGOS_AER091Wp (Pxa2). In preferred embodiments, the polypeptides whose amount is increased are represented by, comprise, essentially consist of, or consist of one, e.g. 2, 3, 4, 5, 6, or more, or all of the amino acid sequences of SEQ ID NO: 1, 3, 5, 7, 9, 11, 13, 15, 17 and/or 19, or homologous sequences thereof as defined herein above.

An over-expression as defined herein above may, in one embodiment, be conveyed by the usage of promoters as defined herein above. Promoters envisaged by the present invention, which may be used for the over-expression of genes as described herein, may either be constitutive promoters, or regulable promoters. It is preferred that the promoters are endogenous, i.e. Eremothecium promoters. In specific embodiments, the promoters may also be heterologous promoters or synthetic promoters, e.g. a strong heterologous promoter, or a regulable heterologous promoter. A promoter may be operably linked with a coding sequence. In a preferred embodiment, the term “promoter” refers to DNA sequence capable of controlling the expression of a coding sequence, which is active in Eremothecium, more preferably in Eremothecium gossypii.

Suitable promoters which may be used in the context of the present invention include the constitutive TEF1 promoter, the constitutive CTS2 promoter, the constitutive RIB3 promoter and the constitutive GPD promoter. Further envisaged examples of suitable promoters include strong constitutive promoters of the glycolysis pathway such as the FBA1, PGK1, or ENO1 promoter, or the strong constitutive RIB4 promoter. Also preferred is the use of the regulable Met3 promoter and the glucose repressible ICL1p promoter. Particularly preferred is the GPD promoter. More preferably, the GPD promoter comprises the sequence according to SEQ ID NO. 68 or a functional fragment thereof which has essentially the same promoter activity as the promoter according to SEQ ID NO. 68.

All of the preferred promoters as mentioned above are endogenous E. gossypii promoters. These promoters may, in specific embodiments, also be used in the context of other organisms of the genus Eremothecium. Further details would be known to the skilled person or can be derived from suitable literature sources such as, for example, Jimenez et al., 2005, Appl Environ Microbol, 71, 5743-5751.

The promoters may be operably linked to genes or sequences to be expressed, as defined herein above.

In a particularly preferred embodiment, an over-expression of the AGOS_ACL174W gene (fat1), the AGOS_AER358C gene (pox1), the AGOS_AGL060W gene (fox2) and/or the AGOS_AFR302W gene (pot1/fox3) is conveyed by a strong promoter. Within the meaning of the present invention, the term “strong promoter” is intended to refer to a promoter the activity of which is higher than the activity of the promoter which is operably linked to the nucleic acid molecule to be overexpressed in a wild-type organism, e.g. a promoter with a higher activity than the promoter of the endogenous fat1, faa1/faa4, pox1, fox 2 or pot1/fox3 gene. Preferably, the activity of the strong promoter is about 2%, 5%, 8%, 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000% or more than 1000% higher than the activity of the promoter which is operably linked to the nucleic acid molecule to be overexpressed in a wild-type organism, e.g. a promoter with a higher activity than the promoter of the endogenous fat1, faa1/faa4, pox1, fox 2 or pot1/fox3 gene. The skilled person knows how to determine the promoter activity and to compare the activities of different promoters. For this purpose, the promoters are typically operably linked to a nucleic acid sequence encoding a reporter protein such as luciferase, green fluorescence protein or beta-glucuronidase and the activity of the reporter protein is determined.

Suitable examples of such strong promoters are the TEF1 promoter, the CTS2 promoter, the RIB3 promoter, the GPD promoter, the FBA1 promoter, the PGK1 promoter, the Met3 promoter, the ICL1 promoter and the RIB4 promoter. Particularly preferred is the GPD promoter. More preferably, the GPD promoter comprises the sequence according to SEQ ID NO. 68 or a functional fragment thereof which has essentially the same promoter activity as the promoter according to SEQ ID NO. 68.

In a further particularly preferred embodiment, an over-expression of the AGOS_ACL174W gene (fat1), the AGOS_AER358C gene (pox1), the AGOS_AGL060W gene (fox2), the AGOS_ABL018C gene (faa1/faa4) and/or the AGOS_AFR302W gene (pot1/fox3) is conveyed by a strong constitutive promoter. Suitable examples of such strong constitutive promoters are the CTS2 promoter, TEF1 promoter, the RIB3 promoter, the GPD promoter, and the RIB4 promoter. Particularly preferred is the GPD promoter. More preferably, the GPD promoter comprises the sequence according to SEQ ID NO. 68 or a functional fragment thereof which has essentially the same promoter activity as the promoter according to SEQ ID NO. 68

In specific embodiments, the promoters may also be heterologous promoters or synthetic promoters, e.g. a strong heterologous promoter, or a regulable heterologous promoter.

An over-expression as defined herein above may, in a further embodiment, be conveyed by the provision of more than one copy of the genetic element to over-expression in the genome. Such second, third, 4th, 5th or further copies of the gene may be completely or almost identical copies of endogenous genetic structures, or they may constitute recombinant modifications thereof. For example, a gene to be over-expressed, e.g. one of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) or AGOS_AER091W (pxa2), may be derived together with its genomic context, preferably including its promoter structure, optionally further comprising 3′ non coding sequences as defined herein above or additional 5′ non-coding sequences as defined herein above, e.g. enhancer elements etc., from the genome of the target Eremothecium organism, or form a close relative, e.g. from E. gossypii if the target is not E. gossypii. Homologous flanks may be used in the range of about 100 to 500 bp. However, also smaller flanks or larger flanks, e.g. up to 1000 bp or more than 1000 bp can in principle be used.

A second or further copy of the gene as mentioned above may subsequently be reintroduced into the organism and be placed in the chromosome. The integration site may be either in vicinity of the original copy, or, preferably, at a different location. The insertion can be preselected via the choice of homologous flanks which are necessary for the integration. The insertion site may accordingly be determined according to known features of the genome, e.g. transcription activity of chromosomal regions, the methylation status of chromosomal regions, potential distance to the first copy (original gene), orientation of the first copy (original gene), the presence of further inserted genes etc. It is preferred that the insertion site is in an intergenic region and/or that transcriptioally active sites are used. In certain embodiments, it is preferred not modifying ORFs and/or regulatory regions of known genes, in particular or essential genes.

In certain embodiments, additional copies may be provided in tandem repeat forms. It is preferred using non-tandem repeats. Due to recombination processes in the genome of Eremothecium it is further preferred keeping the original copy and the second or further copy of a gene as different and/or remote as possible. Such differences may be based on the use of different promoters, the modification of genomic flanks of the genes, or, in specific embodiments, the modification of the nucleotide sequence of the second copy vs. the first copy (original version) of a gene, or a third copy vs. a second copy and/or vs. a first copy (original version) of a gene. Such modification of the nucleotide sequence may be conveyed, for instance, by a modification of the codon-usage of the gene, e.g. as defined herein above. In particular, the codon usage may be modified with the intention to increase or maximize the difference on the nucleotide sequence level, i.e. in order to provide a less similar or the least similar sequence on the nucleotide level, while keeping the amino acid sequence identical or almost identical. In case more than two copies of the same gene shall be introduced into the genome, the codon usage of all copies to be introduced may be adapted such that the difference all copies is maximized, e.g. the difference between original version vs. copy 2 vs. copy 3 is maximized. The same can be done in case of more than 3 copies.

In a particularly preferred embodiment, an over-expression of the AGOS_ACL174W gene (fat1), the AGOS_AER358C gene (pox1), the AGOS_AGL060W gene (fox2), the AGOS_ABL018C gene (faa1/faa4) and/or the AGOS_AFR302W gene (pot1/fox3) is conveyed by the provision of at least a second copy of the AGOS_ACL174W gene (fat1), the AGOS_AER358C gene (pox1), the AGOS_AGL060W gene (fox2), the AGOS_ABL0180 gene (faa1/faa4) and/or the AGOS_AFR302W gene (pot1/fox3) in the genome of Eremothecium.

An over-expression as defined herein above may, in a further embodiment, be conveyed by the an optimization of the codon-usage, e.g. by an adaptation of the codon usage of a gene as defined herein above to the codon usage of the genes which are transcribed or expressed most often in the organism, or which a most highly expressed (in comparison to housekeeping genes such as beta-actin or beta-tubulin). Examples of such codon-usage of highly expressed genes may comprise the codon-usage of a group of the 5, 10, 15, 20, 25 or 30 or more most highly expressed genes of an Eremothecium organism, preferably of E. gossypii.

An over-expression may further be achieved by optimizing the codon usage with respect to the overall codon usage in all or almost all, or 90% or 80% or 75%, or 70% of the transcribed genes of an Eremothecium organism, preferably of E. gossypii. Such an approach may involve an inspection of the codon usage of the gene and a comparison to the overall codon usage as derivable from a genomic sequence of an Eremothecium organism, preferably of E. gossypii, in particular an annotated genomic sequence of the organism, e.g. E. gossypii.

An over-expression may further be achieved by an adaptation of the dicodon-usage, i.e. of the frequency of all two consecutive codons within an ORF. The dicodon-usage of a target gene may accordingly be adapted to the dicodon-usage of highly expressed genes in the organism (in comparison to housekeeping genes such as beta-actin or beta-tubulin). Examples of such dicodon-usage of highly expressed genes may comprise the dicodon-usage of a group of the 5, 10, 15, 20, 25 or 30 or more most highly expressed genes of an Eremothecium organism, preferably of E. gossypii. The adaptation of the dicodon-usage may help to avoid mRNA degrading signals or other transcript portions, which influence the stability of the transcript, since such motives are typically more than 3 nucleotides long and can thus be identified in dicodon, while they may escape attention in codons.

An over-expression may further be achieved by an adaptation of the tricodon-usage, i.e. of the frequency of all two consecutive codons within an ORF. The tricodon-usage of a target gene may accordingly be adapted to the tricodon-usage of highly expressed genes in the organism (in comparison to housekeeping genes such as beta-actin or beta-tubulin). Examples of such tricodon-usage of highly expressed genes may comprise the tricodon-usage of a group of the 5, 10, 15, 20, 25 or 30 or more most highly expressed genes of an Eremothecium organism, preferably of E. gossypii.

Also envisaged is the provision of two codon-modified versions of a target gene, i.e. the original endogenous copy and any further copy may both be modified so that after the modification approach no original version of the gene is present in the genome. This approach may lead to a further distinction of nucleotide sequences and/or increase the expressability or transcription of the target gene(s).

In a particularly preferred embodiment, an over-expression of the AGOS_ACL174W gene (fat1), the AGOS_AER358C gene (pox1), the AGOS_AGL060W gene (fox2), the AGOS_ABL0180 gene (faa1/faa4) and/or the AGOS_AFR302W gene (pot1/fox3) is conveyed by an adaptation of the codon usage, or dicodon usage, or the tricodon usage of a second copy of the AGOS_ACL174W gene (fat1), the AGOS_AER358C gene (pox1), the AGOS_AGL060W gene (fox2), the AGOS_ABL018C gene (faa1/faa4) and/or the AGOS_AFR302W gene (pot1/fox3) in the genome of Eremothecium.

The genetic modification in order to increase the activity of members of the beta-oxidation pathway or of the fatty acid uptake pathway, e.g. the modification leading to an over-expression of genes as mentioned herein above, or below, may be performed by any suitable approach known to the skilled person.

A typical approach which may be used in this context is targeted homologous recombination. For example, a modified version of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) or AGOS_AER091W (pxa2), e.g. a version comprising a constitutive promoter instead of the original prometer or a further copy of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) or AGOS_AER091W (pxa2) comprising the original promoter or a different promoter, e.g. a constitutive promoter as mentioned herein above, may be flanked by DNA homologous to the target endogenous polynucleotide sequence (e.g. the coding regions or regulatory regions of a gene) at whose location the insertion should take place. Such a construct may be used with or without a selectable marker and/or with or without a negative selectable marker, to transform Eremothecium cells. Insertion of the DNA construct, via targeted homologous recombination, results may result in the insertion of a modified version of the targeted gene at the locus of the original gene, or in the insertion of a further copy of the target gene at a different location in the genome. In the latter scenario, the homologous sequences of the place where the second or further copy should be integrated, may be used for the transformation construct. In specific embodiments, homologous transformation may also be used for an inactivation of a gene, e.g. by introducing a resistance marker or other knock out cassette to replace an originally present ORF in the genome.

The term “transformation” refers to the transfer of a genetic element, typically of a nucleic acid molecule, e.g. a specific cassette comprising a construct for homologous recombination, or of extrachromosomal elements such as vectors or plasmids into Eremothecium cells, i.e. into an organism of the genus Eremothecium as defined herein above, wherein said transfer results in a genetically stable inheritance. Conditions for a transformation of Eremothecium cells and corresponding techniques are known to the person skilled in the art. These techniques include chemical transformation, preferably a lithium acetate transformation, as, e.g., derivable from Jimenez et al., 2005, Applied and Environmental Microbiology 71, 5743-5751, protoplast fusion, ballistic impact transformation, electroporation, microinjection, or any other method that introduces the gene or nucleic acid molecule of interest into the fungal cell.

A transformed cell may have at least one copy of the introduced genetic element and may have two or more copies, depending upon where and how the genetic element is integrated into the genome or e.g. in an amplified form. In the context of over-expression constructs it is preferred that the transformation leads to the insertion of a single copy of the over-expression construct or cassette into the genome. Also envisaged is the introduction of two or more copies. Such second or third copies of a specific gene or gene expression construct should preferably be different in terms of their nucleotide sequence from the first copy, while encoding the same amino acid sequence or essentially the same amino acid sequence.

Preferably, the transformed cell may be identified by selection for a marker contained on the introduced genetic element. Alternatively, a separate marker construct may be co-transformed with the desired genetic element, as many transformation techniques introduce many DNA molecules into host cells. Typically, transformed cells may be selected for their ability to grow on selective media. Selective media may incorporate an antibiotic or lack a factor necessary for growth of the untransformed cell, such as a nutrient or growth factor. An introduced marker gene may confer antibiotic resistance, or encode an essential growth factor or enzyme, thereby permitting growth on selective media when expressed in the transformed host. Selection of a transformed cell can also occur when the expressed marker protein can be detected, either directly or indirectly.

The marker protein may be expressed alone or as a fusion to another protein. The marker protein may be detected, for example, by its enzymatic activity. Alternatively, antibodies may be used to detect the marker protein or a molecular tag on, for example, a protein of interest. Cells expressing the marker protein or tag can be selected, for example, visually, or by techniques such as FACS or panning using antibodies. Preferably, any suitable marker that functions in cells of the genus Eremothecium, as known to the person skilled in the art, may be used. More preferably markers which provide resistance to kanamycin, hygromycin, the amino glycoside G418, or nourseothricin (also termed NTC or ClonNAT), as well as the ability to grow on media lacking uracil, leucine, histidine, methionine, lysine or tryptophane may be employed. When using a selection marker as mentioned above, e.g. a G418 or ClonNat resistance marker, or any other suitable marker, sequences of the Cre-lox system may be used in addition to the marker. This system allows upon expression of the Cre recombinase after the insertion of the genetic element, e.g. an over-expression cassette, an elimination and subsequent reuse of the selection marker. Also envisaged is the use of other, similar recombinase systems which would be known the skilled person.

In specific embodiments, markers may also be combined with target sites for site specific nucleases, e.g. ZINC finger nucleases (ZFNs) or meganucleases which are capable of cleaving specific DNA target sequences in vivo. A specific example of such a system is the TALEN (Transcription Activator-Like Effector Nuclease) system, i.e. an artificial restriction enzyme, which is generated by fusing the TAL effector DNA binding domain to a DNA cleavage domain. TAL effectors are proteins which are typically secreted by Xanthomonas bacteria or related species, or which are derived therefrom and have been modified. The DNA binding domain of the TAL effector may comprise a highly conserved sequence, e.g. of about 33-34 amino acid sequence with the exception of the 12th and 13th amino acids which are highly variable (Repeat Variable Diresidue or RVD) and typically show a strong correlation with specific nucleotide recognition. On the basis of this principle, DNA binding domains may be engineered by selecting a combination of repeat segments containing Repeat Variable Diresidue corresponding to an over-expression target gene DNA sequence. The TALEN DNA cleavage domain may be derived from suitable nucleases. For example, the DNA cleavage domain from the FokI endonuclease or from FokI endonuclease variants may be used to construct hybrid nucleases. TALENs may preferably be provided as separate entities due to the peculiarities of the FokI domain, which functions as a dimer.

In specific embodiments, the number of amino acid residues between the TALEN DNA binding domain and the FokI cleavage domain and the number of bases between the two individual TALEN binding sites may be modified or optimized according to the sequence of the construct to be inserted into the Eremothecium genome in order to provide high levels of activity. TALENs or TALEN components may be engineered or modified in order to target any desired DNA sequence, e.g. a DNA sequence comprising a selection marker between homologous ends of a gene to be over-expressed. The enzymatic activity which is required for the recombination may either be provided as such (e.g. similar to the established REMI approach in Eremothecium), or it may be provided together with the selection cassette on the construct, leading to its removal upon the start of the nuclease activity. The engineering may be carried out according to suitable methodologies, e.g. Zhang et al., Nature Biotechnology, 1-6 (2011), or Reyon et al., Nature Biotechnology, 30, 460-465 (2012).

Another system for removing the marker sequences from the genome of the Eremothecium cells is the CRISPR (clustered regularly interspaced short palindromic repeats)/Cas (CRISPR-associated) system which has been shown to facilitate RNA-guided site-specific DNA cleavage and which can be used for genomic engineering (see, e.g., Sander and Young (2014) Nature Biotechnol. 32: 347-355). This system uses Cas9 as a nuclease which is guided by a crRNA and tracrRNA to cleave specific DNA sequences. The mature crRNA:tracrRNA complex directs Cas9 to the target DNA via base-pairing between the spacer on the crRNA and the protospacer on the target DNA next to the protospacer adjacent motif (PAM). Cas9 then mediates the cleavage of the target DNA to create a double-strand break within the protospacer. Instead of crRNA and tracrRNA a guide RNA may be designed to include a hairpin which mimics the tracrRNA-crRNA complex (Jinek et al. (2012) Science 337(6096): 816-821).

In a preferred embodiment of the present invention, the homologous recombination may be carried out as described in the Examples herein below. Particularly preferred is the use of over-expression cassettes comprising a G418 or ClonNAT resistance marker in combination with loxP sequences.

Typically, the genetic elements may be introduced into the Eremothecium cell with the help of a transformation cassette or an expression cassette. In accordance with the present invention the term “transformation cassette” refers to a specific vector containing a foreign gene and having elements in addition to the foreign gene that facilitates transformation of Eremothecium cells. The term “expression cassette” refers to a specific vector containing a foreign gene and having elements in addition to the foreign gene that allow for enhanced expression of that gene in a foreign host, in particular in Eremothecium cells.

Genes of the fatty acid uptake pathway or the beta oxidation pathway as defined herein may accordingly be provided on genetic elements in the form of expression cassettes or transformation cassettes as defined herein above, in particular expression cassettes or transformation cassettes which are prepared for genomic integration via homologous recombination. Also envisaged is the provision on plasmids or vectors. The terms “plasmid” and “vector” refer to an extra chromosomal element often carrying genes that are not part of the central metabolism of the cell, and usually in the form of circular double-stranded DNA fragments. More preferably, the term plasmid refers to any plasmid suitable for transformation of Eremothecium known to the person skilled in the art and in particular to any plasmid suitable for expression of proteins in Eremothecium, e.g. plasmids which are capable of autonomous replication in other organisms, preferably in bacteria, in particular E. coli, and which can be prepared, e.g. digested, for genomic insertional transformation of Eremothecium.

Such expression cassettes or transformation cassettes, or vectors or plasmids may comprise 1, 2, 3, 4, or more or all of the genes or genetic elements involved in the fatty acid uptake pathway and/or the beta oxidation pathway as defined herein above. For example, they may comprise 1, 2, 3, 4, or more or all of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) and AGOS_AER091W (pxa2).

The integration of these cassettes into the genome may occur randomly within the genome or can be targeted through the use of constructs containing regions of homology with the host genome sufficient to target recombination within the host locus, as defined herein above. Where constructs are targeted to an endogenous locus, all or some of the transcriptional and translational regulatory regions may be provided by the endogenous locus. Alternatively, the transcriptional and translational regulatory regions may be provided by the construct.

In case of expression of two or more activities involved in the fatty acid uptake pathway and/or the beta oxidation pathway from separate replicating vectors, it is desirable that each vector or plasmid has a different means of selection and should lack homology to the other constructs to maintain stable expression and prevent reassortment of elements among constructs.

In specific embodiments the genetic elements may comprise microbial expression systems. Such expression systems and expression vectors may contain regulatory sequences that direct high level expression of foreign proteins.

In a preferred embodiment of the present invention a genetically modified organism as defined herein above, e.g. an organism which comprises a modification of a genetic element associated with the fatty acid uptake, and/or a genetic element associated with the beta-oxidation pathway, e.g. an organism in which one or more of the genes AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) and AGOS_AER091W (pxa2) is/are over-expressed, and/or in which one or more of the polypeptides of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and/or AGOS_AER091Wp (Pxa2) is/are provided in an increased amount, and/or in which one or more of the activities of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and/or AGOS_AER091Wp (Pxa2) is/are increased, is capable of accumulating more riboflavin than a comparable organism without the genetic modification. The term “comparable organism” as used herein refers to an organism with the same or a very similar genetic background as the organism which is used as starting organism for the genetic modification. Preferably, a comparable organism may be an organism used for the genetic modifications as described herein. If the genetic modification is performed in a wildtype organism, the wildtype organism may be considered as comparable organism. In further embodiments, any wildtype organism may be considered as comparable organism if the genetic modification is performed in any other or the same wildtype organism. If the genetic modification is performed in a riboflavin overproducing organism or strain as defined herein above, said riboflavin overproducing organism without the genetic modification may be considered as comparable organism.

The genetic modification(s) as described herein may lead to an increase of the amount of riboflavin produced or accumulated by the organism. The increase may, in specific embodiments, depend on the genetic background of the organism in which the modifications are performed, and/or on the number of modifications, and/or the type of over-expression technique, and/or the copy number present and/or other factors such as the culture conditions, culture medium conditions etc., or on a combination of any of the above parameters and factors. For example, the increase may be at least 0.3%, 0.5%, 0.7%, 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, or more than 100% compared to an organism not having the genetic modification which is cultured under the same conditions as the genetically modified organism of the present invention.

The determination of the riboflavin production or accumulation and thus also of the increase of this production in the modified organisms in comparison to comparable organisms may be performed as described above, i.e. by following a cell culture riboflavin determination protocol based on specific culture conditions and the use of a nicotinamide based photometric assay as described herein above. In specific embodiments, the determination may be performed as described in the Examples provided below. The present invention also envisages further determination protocols or procedures, including protocols or improvements of protocols which may be developed in the future.

In a further embodiment the present invention relates to a genetically modified organism as defined herein above or a method for the production or accumulation of riboflavin using said genetically modified organism, wherein said organism preferably comprises a genetic modification which leads to an over-expression of at least one of AGOS_ACL174W (fat1), AGOS_ABL018C (faa1/faa4), AGOS_AER358C (pox1), AGOS_AGL060W (fox2), AGOS_AFR302W (pot1/fox3), AGOS_AFL213W, AGOS_ADR1650, AGOS_AFR453W (pex5), AGOS_ACR128C (pxa1) or AGOS_AER091W (pxa2), and/or in which at least one of the polypeptides of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and AGOS_AER091Wp (Pxa2) is provided in an increased amount, and/or in which at least one of the activities of AGOS_ACL174Wp (Fat1), AGOS_ABL018Cp (Faa1/Faa4), AGOS_AER358Cp (Pox1), AGOS_AGL060Wp (Fox2), AGOS_AFR302Wp (Pot1/Fox3), AGOS_AFL213Wp, AGOS_ADR165Cp, AGOS_AFR453Wp (Pex5), AGOS_ACR128Cp (Pxa1) and AGOS_AER091Wp (Pxa2), is increased, preferably as defined in detail herein above, and wherein said organism comprises at least one additional genetic modification.

The term “additional genetic modification” as used herein refers to any further genetic or biochemical modification of an organism as defined above, e.g. a modification such as a deletion of a gene or genomic region, the over-expression of a gene or gene fragment etc.

In a preferred embodiment, the additional genetic modification of an organism as defined above, concerns elements which have an influence on the production of riboflavin. Such elements may already be known or may be found in the future. Preferably, the additional genetic modification may concern an activity which has known influence on the production of riboflavin in Eromothecium, more preferably in E. gossypii. Examples of activities which are known to have such an influence comprise GLY1; SHM2; ADE4; PRS 2, 4; PRS 3; MLS1; BAS1; RIB 1; RIB 2; RIB 3; RIB 4; RIB 5; GUA1; ADE12; IMPDH; and RIB 7.

Accordingly, genetic modifications may be carried out with one or more of the genes gly1; shm2; ade4; prs 2, 4; prs 3; mls1; bas1; rib 1; rib 2; rib 3; rib 4; rib 5; gua1; ade12; impdh; and/or rib 7 of Eremothecium, preferably of E. gossypii.

In further preferred embodiments, the additional genetic modification may results in at least one of the following alterations: (i) the GLY1 activity is increased; and/or (ii) the SHM2 activity is decreased or eliminated; and/or (iii) the ADE4 activity is increased and/or provided as feedback-inhibition resistant version; and/or (iv) the PRS 2, 4 activity is increased; and/or (v) the PRS 3 activity is increased; and/or (vi) the MLS1 activity is increased; and/or (vii) the BAS1 activity is decreased or eliminated; and/or (viii) the RIB 1 activity is increased; and/or (ix) the RIB 2 activity is increased; and/or (x) the RIB 3 activity is increased; and/or (xi) the RIB 4 activity is increased; and/or (xii) the RIB 5 activity is increased; and/or (xiii) the RIB 7 activity is increased; and/or (xiv) the GUA 1 activity is increased; and/or (xv) the ADE12 activity is decreased; and/or (xvi) the IMPDH activity is increased.

In further preferred embodiments, the activity of AGOS_AFR366Wp (GLY1) is provided by a polypeptide comprising, essentially consisting of or consisting of the amino acid sequence of SEQ ID NO: 21 or functional parts or fragments thereof, or is encoded by a nucleic acid comprising, essentially consisting of or consisting of the nucleotide sequence of SEQ ID NO: 22 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 21 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 22 or functional parts or fragments thereof.

In further preferred embodiments, the activity of AGOS_AEL188Wp (SHM2) is provided by a polypeptide comprising, essentially consisting of or consisting of the amino acid sequence of SEQ ID NO: 23 or functional parts or fragments thereof, or is encoded by a nucleic acid comprising, essentially consisting of or consisting of the nucleotide sequence of SEQ ID NO: 24 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 23 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 24 or functional parts or fragments thereof.

In further preferred embodiments, the activity of AGOS_AGL334Wp (ADE4) is provided by a polypeptide comprising, essentially consisting of or consisting of the amino acid sequence of SEQ ID NO: 25 or functional parts or fragments thereof, or is encoded by a nucleic acid comprising, essentially consisting of or consisting of the nucleotide sequence of SEQ ID NO: 26 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 25 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 26 or functional parts or fragments thereof.

In further preferred embodiments, the activity of AGOS_AGR371Cp (PRS 2, 4) is provided by a polypeptide comprising, essentially consisting of or consisting of the amino acid sequence of SEQ ID NO: 27 or functional parts or fragments thereof, or is encoded by a nucleic acid comprising, essentially consisting of or consisting of the nucleotide sequence of SEQ ID NO: 28 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 27 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 28 or functional parts or fragments thereof.

In further preferred embodiments, the activity of AGOS_AGL080Cp (PRS 3) is provided by a polypeptide comprising, essentially consisting of or consisting of the amino acid sequence of SEQ ID NO: 29 or functional parts or fragments thereof, or is encoded by a nucleic acid comprising, essentially consisting of or consisting of the nucleotide sequence of SEQ ID NO: 30 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 29 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 30 or functional parts or fragments thereof.

In further preferred embodiments, the activity of AGOS_ACR268Cp (MLS1) is provided by a polypeptide comprising, essentially consisting of or consisting of the amino acid sequence of SEQ ID NO: 31 or functional parts or fragments thereof, or is encoded by a nucleic acid comprising, essentially consisting of or consisting of the nucleotide sequence of SEQ ID NO: 32 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 31 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 32 or functional parts or fragments thereof.

In further preferred embodiments, the activity of AGOS_AFR297Wp (BAS1) is provided by a polypeptide comprising, essentially consisting of or consisting of the amino acid sequence of SEQ ID NO: 33 or functional parts or fragments thereof, or is encoded by a nucleic acid comprising, essentially consisting of or consisting of the nucleotide sequence of SEQ ID NO: 34 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 33 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 34 or functional parts or fragments thereof.

In further preferred embodiments, the activity of AGOS_ADL296Cp (RIB1) is provided by a polypeptide comprising, essentially consisting of or consisting of the amino acid sequence of SEQ ID NO: 35 or functional parts or fragments thereof, or is encoded by a nucleic acid comprising, essentially consisting of or consisting of the nucleotide sequence of SEQ ID NO: 36 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 35 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 36 or functional parts or fragments thereof.

In further preferred embodiments, the activity of AGOS_AEL091Cp (RIB2) is provided by a polypeptide comprising, essentially consisting of or consisting of the amino acid sequence of SEQ ID NO: 37 or functional parts or fragments thereof, or is encoded by a nucleic acid comprising, essentially consisting of or consisting of the nucleotide sequence of SEQ ID NO: 38 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 37 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 38 or functional parts or fragments thereof.

In further preferred embodiments, the activity of AGOS_ADR118Cp (RIB3) is provided by a polypeptide comprising, essentially consisting of or consisting of the amino acid sequence of SEQ ID NO: 39 or functional parts or fragments thereof, or is encoded by a nucleic acid comprising, essentially consisting of or consisting of the nucleotide sequence of SEQ ID NO: 40 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 39 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 40 or functional parts or fragments thereof.

In further preferred embodiments, the activity of AGOS_AGR396Wp (RIB4) is provided by a polypeptide comprising, essentially consisting of or consisting of the amino acid sequence of SEQ ID NO: 41 or functional parts or fragments thereof, or is encoded by a nucleic acid comprising, essentially consisting of or consisting of the nucleotide sequence of SEQ ID NO: 42 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 41 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 42 or functional parts or fragments thereof.

In further preferred embodiments, the activity of AGOS_AGR241Wp (RIB5) is provided by a polypeptide comprising, essentially consisting of or consisting of the amino acid sequence of SEQ ID NO: 43 or functional parts or fragments thereof, or is encoded by a nucleic acid comprising, essentially consisting of or consisting of the nucleotide sequence of SEQ ID NO: 44 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 43 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 44 or functional parts or fragments thereof.

In further preferred embodiments, the activity of AGOS_AER037Cp (RIB7) is provided by a polypeptide comprising, essentially consisting of or consisting of the amino acid sequence of SEQ ID NO: 45 or functional parts or fragments thereof, or is encoded by a nucleic acid comprising, essentially consisting of or consisting of the nucleotide sequence of SEQ ID NO: 46 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 45 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 46 or functional parts or fragments thereof.

In further preferred embodiments, the GUA1 activity is provided by a polypeptide comprising, essentially consisting of or consisting of the amino acid sequence of SEQ ID NO: 69 or functional parts or fragments thereof, or is encoded by a nucleic acid comprising, essentially consisting of or consisting of the nucleotide sequence of SEQ ID NO: 70 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 69 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 70 or functional parts or fragments thereof.

In further preferred embodiments, the ADE12 activity is provided by a polypeptide comprising, essentially consisting of or consisting of the amino acid sequence of SEQ ID NO: 71 or functional parts or fragments thereof, or is encoded by a nucleic acid comprising, essentially consisting of or consisting of the nucleotide sequence of SEQ ID NO: 72 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 71 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 72 or functional parts or fragments thereof.

In further preferred embodiments, the IMPDH activity is provided by a polypeptide comprising, essentially consisting of or consisting of the amino acid sequence of SEQ ID NO: 73 or functional parts or fragments thereof, or is encoded by a nucleic acid comprising, essentially consisting of or consisting of the nucleotide sequence of SEQ ID NO: 74 or functional parts or fragments thereof, or is provided by a polypeptide comprising, essentially consisting of or consisting of an amino acid having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the amino acid sequence of SEQ ID NO: 73 or functional parts or fragments thereof, or is encoded by a nucleic acid comprising, essentially consisting of or consisting of a nucleotide sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleotide sequence of SEQ ID NO: 74 or functional parts or fragments thereof.

The term “functional parts or fragments thereof” as used in the context of sequences described herein refers to sections or parts of the polypeptide and the encoding nucleotide sequence, which are able to perform a specific enzymatic reaction.

In specific embodiments, the AGOS_AFR366Wp (GLY1) activity and (i) the AGOS_AGL334Wp (ADE4) activity, and/or (ii) the AGOS_AGR371 Cp (PRS 2, 4) activity, and/or (iii) the AGOS_AGL080Cp (PRS 3) activity, and/or the (iv) AGOS_ACR268Cp (MLS1) activity and/or the (v) AGOS_AER350W (GUA1) activity and/or the (vi) AGOS_AER117W (IMPDH) activity may be increased.

In further specific embodiments, the AGOS_AFR366Wp (GLY1) activity and (i) the AGOS_ADL296Cp (RIB1) activity, or (ii) the AGOS_AEL091Cp (RIB2) activity, or (iii) the AGOS_ADR118Cp (RIB3) activity, or the (iv) AGOS_AGR396Wp (RIB4) activity, or the (v) AGOS_AGR241Wp (RIB5) activity, or the (vi) AGOS_AER037Cp (RIB7) activity may be increased.

In further specific embodiments, the AGOS_AFR366Wp (GLY1) activity may be increased and (i) the AGOS_AEL188Wp (SHM2) activity may be decreased or eliminated, and/or the (ii) AGOS_AFR297Wp (BAS1) activity may be decreased or eliminated, and/or the (iii) AGOS_ABL186W (ADE12) activity may be decreased or eliminated.

In yet another set of embodiments, the AGOS_AFR366Wp (GLY1) activity and (i) the AGOS_AGL334Wp (ADE4) activity, and/or (ii) the AGOS_AGR371Cp (PRS 2, 4) activity, and/or (iii) the AGOS_AGL080Cp (PRS 3) activity, and/or the (iv) AGOS_ACR268Cp (MLS1) activity may be increased. In yet another set of embodiments, the AGOS_AFR366Wp (GLY1) activity and the (i) AGOS_ADL296Cp (RIB1) activity, and/or (ii) the AGOS_AEL091Cp (RIB2) activity and/or or (iii) the AGOS_ADR118Cp (RIB3) activity, and/or the (iv) AGOS_AGR396Wp (RIB4) activity, and/or the (v) AGOS_AGR241Wp (RIB5) activity, and/or the (vi) AGOS_AER037Cp (RIB7) activity may be increased.

In yet another set of embodiments, the AGOS_AFR366Wp (GLY1) activity and (i) the AGOS_AGL334Wp (ADE4) activity, and/or (ii) the AGOS_AGR371Cp (PRS 2, 4) activity, and/or (iii) the AGOS_AGL080Cp (PRS 3) activity, and/or the (iv) AGOS_ACR268Cp (MLS1) activity and/or the (v) AGOS_ADL296Cp (RIB1) activity, and/or (vi) the AGOS_AEL091Cp (RIB2) activity and/or or (vii) the AGOS_ADR118Cp (RIB3) activity, and/or the (viii) AGOS_AGR396Wp (RIB4) activity, and/or the (ix) AGOS_AGR241Wp (RIB5) activity, and/or the (x) AGOS_AER037Cp (RIB7) activity may be increased.

In yet another set of embodiments, the AGOS_AFR366Wp (GLY1) activity and (i) the AGOS_AGL334Wp (ADE4) activity, and/or (ii) the AGOS_AGR371Cp (PRS 2, 4) activity, and/or (iii) the AGOS_AGL080Cp (PRS 3) activity, and/or the (iv) AGOS_ACR268Cp (MLS1) activity and/or the (v) AGOS_ADL296Cp (RIB1) activity, and/or (vi) the AGOS_AEL091Cp (RIB2) activity and/or or (vii) the AGOS_ADR118Cp (RIB3) activity, and/or the (viii) AGOS_AGR396Wp (RIB4) activity, and/or the (ix) AGOS_AGR241Wp (RIB5) activity, and/or the (x) AGOS_AER037Cp (RIB7) activity may be increased and/or the (x) the AGOS_AEL188Wp (SHM2) activity may be decreased or eliminated, and/or the (xi) AGOS_AFR297Wp (BAS1) activity may be decreased or eliminated and/or the (xii) AGOS_ABL186W (ADE12) activity may be decreased or eliminated.

In yet another set of embodiments, the AGOS_AFR366Wp (GLY1) activity and (i) the AGOS_AGL334Wp (ADE4) activity, and (ii) the AGOS_AGR371 Cp (PRS 2, 4) activity, and (iii) the AGOS_AGL080Cp (PRS 3) activity may be increased.

In yet another set of embodiments, the AGOS_AFR366Wp (GLY1) activity and (i) the AGOS_AGL334Wp (ADE4) activity, and (ii) the AGOS_AGR371 Cp (PRS 2, 4) activity, and (iii) the AGOS_AGL080Cp (PRS 3) activity may be increased and the (iv) the AGOS_AEL188Wp (SHM2) activity may be decreased or eliminated, and the (v) AGOS_AFR297Wp (BAS1) activity may be decreased or eliminated.

In yet another set of embodiments, the AGOS_AFR366Wp (GLY1) activity and the (i) AGOS_ADL296Cp (RIB1) activity, and (ii) the AGOS_AEL091Cp (RIB2) activity and (iii) the AGOS_ADR118Cp (RIB3) activity, and the (iv) AGOS_AGR396Wp (RIB4) activity, and the (v) AGOS_AGR241Wp (RIB5) activity, and the (vi) AGOS_AER037Cp (RIB7) activity may be increased.

In yet another set of embodiments, the AGOS_AFR366Wp (GLY1) activity and the (i) AGOS_ADL296Cp (RIB1) activity, and (ii) the AGOS_AEL091Cp (RIB2) activity and (iii) the AGOS_ADR118Cp (RIB3) activity, and the (iv) AGOS_AGR396Wp (RIB4) activity, and the (v) AGOS_AGR241Wp (RIB5) activity, and the (vi) AGOS_AER037Cp (RIB7) activity may be increased and the (vii) the AGOS_AEL188Wp (SHM2) activity may be decreased or eliminated, and the (viii) AGOS_AFR297Wp (BAS1) activity may be decreased or eliminated.

In yet another set of embodiments, the AGOS_AFR366Wp (GLY1) activity and (i) the AGOS_AGL334Wp (ADE4) activity, and (ii) the AGOS_AGR371 Cp (PRS 2, 4) activity, and (iii) the AGOS_AGL080Cp (PRS 3) activity may be increased and the (iv) AGOS_ACR268Cp (MLS1) activity and the (v) AGOS_ADL296Cp (RIB1) activity, and (vi) the AGOS_AEL091Cp (RIB2) activity and (vii) the AGOS_ADR118Cp (RIB3) activity, and the (viii) AGOS_AGR396Wp (RIB4) activity, and the (ix) AGOS_AGR241Wp (RIB5) activity, and the (x) AGOS_AER037Cp (RIB7) activity may be increased.

In yet another set of embodiments, the AGOS_AFR366Wp (GLY1) activity and (i) the AGOS_AGL334Wp (ADE4) activity, and (ii) the AGOS_AGR371 Cp (PRS 2, 4) activity, and (iii) the AGOS_AGL080Cp (PRS 3) activity may be increased and the (iv) AGOS_ACR268Cp (MLS1) activity and the (v) AGOS_ADL296Cp (RIB1) activity, and (vi) the AGOS_AEL091Cp (RIB2) activity and (vii) the AGOS_ADR118Cp (RIB3) activity, and the (viii) AGOS_AGR396Wp (RIB4) activity, and the (ix) AGOS_AGR241Wp (RIB5) activity, and the (x) AGOS_AER037Cp (RIB7) activity may be increased and the (x) the AGOS_AEL188Wp (SHM2) activity may be decreased or eliminated, and the (xi) AGOS_AFR297Wp (BAS1) activity may be decreased or eliminated.

The increase of the activity of AGOS_AFR366Wp (GLY1) may be due to an over-expression of the AGOS_AFR366W (gly1) gene. An increase of the activity of AGOS_AGL334Wp (ADE4) may be due to an over-expression of the AGOS_AGL334W (ade4) gene. An increase of the activity of AGOS_AGR371 Cp (PRS 2, 4) may be due to an over-expression of the AGOS_AGR371C (prs 2, 4) gene. An increase of the activity of AGOS_AGL080Cp (PRS 3) may be due to an over-expression of the AGOS_AGL080C (prs 3) gene. An increase of the activity of AGOS_ACR268Cp (MLS1) may be due to an over-expression of the AGOS_ACR268C (mls1) gene. An increase of the activity of AGOS_ADL296Cp (RIB1) may be due to an over-expression of the AGOS_ADL296C (rib1) gene. An increase of the activity of AGOS_AEL091 Cp (RIB2 may be due to an over-expression of the AGOS_AEL091C (rib2) gene. An increase of the activity of AGOS_ADR118Cp (RIB3) may be due to an over-expression of the AGOS_ADR118Cp (rib3) gene. An increase of the activity of AGOS_AGR396Wp (RIB4) may be due to an over-expression of the AGOS_AGR396W (rib4) gene. An increase of the activity of AGOS_AGR241Wp (RIB5) may be due to an over-expression of the AGOS_AGR241W (rib5) gene. An increase of the activity of AGOS_AER037Cp (RIB7) may be due to an over-expression of the AGOS_AER037C (rib7) gene. A decrease or elimination of the activity of AGOS_AEL188Wp (SHM2) may be due to an inactivation of the AGOS_AEL188W (shm2) gene. A decrease or elimination of the activity of AGOS_AFR297Wp (BAS1) may be due to an inactivation of the AGOS_AFR297W (bas1) gene. The increase of the GUA1 activity may be due to an over-expression of the AGOS_AER350W (gua 1) gene. The increase of the IMPDH activity may be due to an over-expression of the AGOS_AER117W (impdh) gene. A decrease or elimination of the ADE12 activity may be due to an inactivation of the AGOS_ABL186W (ade12) gene.

The over-expression of the AGOS_AFR366W (gly1) gene, the AGOS_AGL334W (ade4) gene, the AGOS_AGR371C (prs 2, 4) gene, the AGOS_AGL080C (prs 3) gene, the AGOS_ACR268C (mls1) gene, the AGOS_ADL296C (rib1) gene, the AGOS_AEL091C (rib2) gene, the AGOS_ADR118Cp (rib3) gene, the AGOS_AGR396Wp (rib4) gene, the AGOS_AGR241W (rib5) gene, the AGOS_AER350W (gua 1) gene, the AGOS_AER117W (impdh) gene and/or the AGOS_AER037C (rib7) gene may be carried out according to approaches, methods and processes as outlined herein above, preferably by using strong promoters, e.g. constitutive promoter such as the GDP promoter. In specific embodiments, the promoter may also be a heterologous promoter or a synthetic promoter, e.g. a strong heterologous promoter, or a regulable heterologous promoter.

The term “inactivation” or as used herein refers to a modification of the genetic element encoding an enzymatic activity, e.g. on a molecular basis, the transcript expressed by the genetic element or the protein or enzymatic activity encoded by said genetic element, which leads to a complete or partial cease of functioning of the activity. A partial inactivation or partial cease of functioning of the activity may, for example, lead to a residual enzymatic activity of about 95%, 90%, 85%, 80%, 75%, 75%, 70%, 65%, 60%, 55%, 50%, 45%, 40%, 35%, 30%, 25%, 20%, 15%, 10%, 5%, 3% or less than 3% or any value in between the mentioned values of the wildtype or full enzymatic activity. Examples of an envisaged inactivation are a functional disruption or deletion of at least one genomic copy, preferably all genomic copies, of at least one of AGOS_AEL188W (SHM2), AGOS_ABL186W (ADE12) and AGOS_AFR297W (BAS1). In preferred embodiments, the genetic elements or genomic copies to be deleted are, comprise, partially comprise, essentially consist of or consist of the nucleotide sequences of SEQ ID NO: 24, 72 and/or 34, or homologous sequences thereof as defined herein above. The deletion may encompass any region of two or more residues in a coding (ORF) or non-coding portion of the genetic element, e.g. from two residues up to the entire gene or locus. In specific embodiments deletions may also affect smaller regions, such as domains, protein sub-portions, repeated sequences or fragments of less than about 50 consecutive base pairs, although larger deletions may occur as well. The deletion may comprise regions of one protein subunit or more than one protein subunit, e.g. in cases in which the protein or enzyme is composed of several subunits. The deletion or functional disruption preferably takes place within the coding sequence or ORF of AGOS_AEL188W (SHM2), AGOS_ABL186W (ADE12) or AGOS_AFR297W (BAS1). Also envisaged is a functional disruption in the 3′ non-coding sequence of the AGOS_AEL188W (SHM2), AGOS_ABL186W (ADE12) or AGOS_AFR297W (BAS1) gene, as defined herein above, in the promoter sequence (also 5′ non coding region) of AGOS_AEL188W (SHM2), AGOS_ABL186W (ADE12) or AGOS_AFR297W (BAS1), as defined herein above, or in a regulatory sequence associated with AGOS_AEL188W (SHM2), AGOS_ABL186W (ADE12) or AGOS_AFR297W (BAS1), as defined herein above. Such functional disruptions or modifications may lead, for example, to a decrease of expression or an instability of the transcript, difficulties in transcription initiation etc. thus providing a reduced amount or complete absence of the enzymatic activity. In further embodiments, the inactivation may also be due to a mutation, rearrangement and/or insertion in the coding (ORF) and/or non-coding region of the genetic elements of AGOS_AEL188W (SHM2), AGOS_ABL186W (ADE12) or AGOS_AFR297W (BAS1), e.g. in the regulatory sequences. Mutations may, for example, be point mutations or 2- or 3-nucleotide exchanges, which lead to a modification of the encoded amino acid sequence, or the introduction of one or more frame-shifts into the ORF, or the introduction of premature stop codons, or the removal of stop codons from the ORF, and/or the introduction of recognition signals for cellular machineries, e.g. the polyadenylation machinery or the introduction of destruction signals for protein degradation machineries etc. Such modified sequence portions may give rise to proteins which no longer provide the activity of the protein's wildtype version. The proteins may accordingly, for example, have substitutions in relevant enzymatic core regions, leading to a cessation of functioning, or may be composed of different amino acids (due to frameshifts) and thus be unable to function properly. The modified sequence portions may further give rise to unstable transcripts, which are prone to degradation. Furthermore, the targeting of the proteins may be compromised.

The functional disruption or deletion of genetic elements n, as well as the introduction of point mutations in these genetic elements as outlined above may be performed by any suitable approach known to the skilled person, e.g. by homologous recombination as described herein above.

In further specific embodiments, the inactivation may be due to specific inactivation processes taking place on the level of RNA transcripts. Such inactivation may be due to sequence specific recognition of RNA transcripts of AGOS_AEL188W (SHM2), AGOS_ABL186W (ADE12) or AGOS_AFR297W (BAS1) and a subsequent degradation of these transcripts. For this approach RNA interference or antisense methods as known from higher eukaryotes may be used. Although fungi such as Eremothecium are assumed to lack the necessary activities for RNAi, the present invention envisages the introduction of required activities by genetic engineering. An example, how RNAi can be established for Eremothecium in analogy to the situation of S. cerevisiae is derivable from Drinnenberg et al, 2009, Science 326 (5952), 544-550. Accordingly, the present invention envisages the provision of siRNA species which are specific for any one of the transcripts of AGOS_AEL188W (SHM2), ACOS_ABL186W (ADE12) or AGOS_AFR297W (BAS1), or a combination thereof.

The term “siRNA” refers to a particular type of antisense-molecules, i.e. a small inhibitory RNA duplexes that induce the RNA interference (RNAi) pathway. These molecules can vary in length and may be between about 18-28 nucleotides in length, e.g. have a length of 18, 19, 20, 21, 22, 23, 24, 25, 26, 27 or 28 nucleotides. Preferably, the molecule has a length of 21, 22 or 23 nucleotides. The siRNA molecule according to the present invention may contain varying degrees of complementarity to their target mRNA, preferably in the antisense strand. siRNAs may have unpaired overhanging bases on the 5′ or 3′ end of the sense strand and/or the antisense strand. The term “siRNA” includes duplexes of two separate strands, as well as single strands that can form hairpin structures comprising a duplex region. Preferably the siRNA may be double-stranded wherein the double-stranded siRNA molecule comprises a first and a second strand, each strand of the siRNA molecule is about 18 to about 23 nucleotides in length, the first strand of the siRNA molecule comprises nucleotide sequence having sufficient complementarity to the target RNA via RNA interference, and the second strand of said siRNA molecule comprises nucleotide sequence that is complementary to the first strand. The production of such interference molecules may further be controlled and regulated via the production of siRNAs from regulable promoters.

In yet another specific embodiment of the present invention, the inactivation may be due to specific inactivation processes taking place on the level of proteins or enzymes. This inactivation may be due to a binding of specifically binding molecules such as small molecules to the enzyme or protein of AGOS_AEL188Wp (SHM2), ACOS_ABL186W (ADE12) or AGOS_AFR297Wp (BAS1).

A “small molecules” in the context of the present invention refers to a small organic compound that is preferably biologically active, i.e. a biomolecule, but is preferably not a polymer. Such an organic compound may have any suitable form or chemical property. The compound may be a natural compound, e.g. a secondary metabolite or an artificial compound, which has been designed and generated de novo. In one embodiment of the present invention a small molecule is capable of blocking the binding AGOS_AEL188Wp (SHM2), AGOS_ABL186W (ADE12) or AGOS_AFR297Wp (BAS1) to substrates, or capable of blocking the activity of AGOS_AEL188Wp (SHM2), AGOS_ABL186W (ADE12) or AGOS_AFR297Wp (BAS1). For example, a small molecule may bind to AGOS_AEL188Wp (SHM2), AGOS_ABL186W (ADE12) or AGOS_AFR297Wp (BAS1) and thereby induce a tight or irreversible interaction between the molecule and the protein, thus leading to a cessation or compromising of the normal (wildtype) function of the protein or enzyme, e.g. if the enzymatic core or binding pocket is involved.

Methods and techniques for the identification and preparation of such small molecules as well as assays for the testing of small molecules are known to the person skilled in the art and also envisaged herein.

In a further preferred embodiment, the activity of AGOS_AGL334Wp (ADE4) an feedback inhibited version of ADE4, which is provided by a polypeptide comprising, essentially consisting of or consisting of the amino acid sequence of SEQ ID NO: 47 or functional parts or fragments thereof, or is encoded by a nucleic acid comprising, essentially consisting of or consisting of the nucleotide sequence of SEQ ID NO: 48 or functional parts or fragments thereof, or is provided by a polypeptide comprising, essentially consisting of or consisting of an amino acid having at least about 95%, 96%, 97%, 98%, 99%, or more sequence identity to the amino acid sequence of SEQ ID NO: 47 or functional parts or fragments thereof, or is encoded by a nucleic acid comprising, essentially consisting of or consisting of a nucleotide sequence having at least about 95%, 96%, 97%, 98%, 99%, or more sequence identity to the nucleotide sequence of SEQ ID NO: 48 or functional parts or fragments thereof. Further details on the feedback inhibited version of ADE4 may be derived from Jimenez et al., 2005, Applied Environmental Microbiology 71, 5743-5751.

The present invention further envisages the use of genes encoding activities involved in the fatty acid uptake and/or beta oxidation pathway as defined herein above for the increasing the accumulation of riboflavin in the an organism of the genus Eremothecium. The genes to be used for this approach may any of the genes mentioned herein above, e.g. the AGOS_ACL174W gene (fat1), the AGOS_AER358C gene (pox1), the AGOS_AGL060W gene (fox2) and/or the AGOS_AFR302W gene (pot1/fox3). In further embodiments, additional genes such as AGOS_ABL0180 (faa1/faa4), AGOS_AFL213W, AGOS_ADR1650, AGOS_AFR453W (pex5), AGOS_ACR128C (pxa1) or AGOS_AER091W (pxa2) may be used for the accumulation of riboflavin in an organism of the genus Eremothecium. The mentioned genes may be used such that the encoded polypeptides and activities may be provided in an increased amount or concentration in the cells. The genes may be used in any suitable combination or linking, preferably as described herein above. It is preferred that at least AGOS_ACL174W gene (fat1), the AGOS_AER358C gene (pox1), the AGOS_AGL060W gene (fox2) and/or the AGOS_AFR302W gene (pot1/fox3) be over-expressed. In certain embodiments, the increasing the accumulation of riboflavin may include also the production of riboflavin, e.g. as defined herein above.

In further specific embodiments, additional genes may used for increasing the accumulation of riboflavin in an organism of the genus Eremothecium. These genes may include gly1; shm2; ade4; prs 2, 4; prs 3; mls1; bas1; rib 1; rib 2; rib 3; rib 4; rib 5; gua1; ade12; impdh and/or rib 7 of Eremothecium, preferably of E. gossypii as defined herein above. It is particularly preferred that gly1 is over-expressed so that the GLY1 activity is increased; that shm2 is inactivated so that the SHM2 activity is decreased or eliminated; that ade4 is over-expressed, or that an ade4 feedback resistant mutant is expressed or over-expressed so that the ADE4 activity is increased and/or provided as feedback-inhibition resistant version; that prs 2, 4 is over-expresses so that the PRS 2, 4 activity is increased; that mls1 is over-expressed so that the MLS1 activity is increase; that bas1 is inactivated so that the BAS1 activity is decreased or eliminated; that rib 1 is over-expressed so that the RIB 1 activity is increased; that rib 2 is over-expressed so that the RIB 2 activity is increased; that rib 3 is over-expressed so that the RIB 3 activity is increased; that rib 4 is over-expressed so that the RIB 4 activity is increased; that rib 5 is over-expressed so that the RIB 5 activity is increased; that gua1 is over-expressed so that the GUA1 activity is increased; that ade12 is inactivated so that the ADE12 activity is decreased; that impdh is over-expressed so that the IMPDH activity is increased; and/or that rib 7 is over-expressed so that the RIB 7 activity is increased. In specific embodiments, these genes may be over-expressed or provided in the form as defined herein above, e.g. in different combinations and amounts.

The organism may be any Eremothecium species as described herein above, preferably Eremothecium gossypii. The use of Eremothecium for increasing the accumulation of riboflavin may comprise the use of suitable fermentation environments, nutrition, riboflavin extraction from the fermentation vessels etc. The present invention accordingly envisages a corresponding method for the production of riboflavin, or derivatives thereof as defined herein above. In specific embodiments, the Eremothecium species is an organism which is capable of accumulating already 50 to 100 mg/I culture medium riboflavin, more preferably more than 50 to 100 mg/I culture medium riboflavin. In further embodiments, the Eremothecium species may be an organism which is has been genetically modified. The genetic modification may be a modification as described herein, e.g. have a direct influence on the production or accumulation of riboflavin, or may have different effects, e.g. in other pathways, or concern the production of other biochemical entities in addition to riboflavin such as PUFAs, fatty acids, amino acids, sugars etc., concern the possibilities of using certain carbon sources, concern the possibilities of using certain nitrogen sources etc., concern the stability of the genome or of genomic regions, allow for or improve steps of homologous recombination, allow for the expression of heterologous genes or promoters etc., improve culture behavior of the cells such as filamentation, mycel fragmentation, pH tolerance, density tolerance, use of salts, salt tolerance, concern the generation rate of the cells, concern the resistance towards antibiotics or any other trait which could be advantageous for the production of riboflavin or the co-production of riboflavin and another product.

The present invention further envisages the use of genes encoding activities involved in the fatty acid uptake and/or beta oxidation pathway as defined herein above for the increasing the accumulation of riboflavin in the an organism of the genus Eremothecium. The genes to be used for this approach may any of the genes mentioned herein above, e.g. the AGOS_ACL174W gene (fat1), the AGOS_AER358C gene (pox1), the AGOS_AGL060W gene (fox2), the AGOS_ABL018C gene (faa1/faa4) and/or the AGOS_AFR302W gene (pot1/fox3)

In a particularly preferred embodiment the AGOS_ACL174W gene (fat1), the AGOS_AER358C gene (pox1), the AGOS_AGL060W gene (fox2), the AGOS_ABL018C gene (faa1/faa4) and/or the AGOS_AFR302W gene (pot1/fox3) may be used such that they are over-expressed via a strong, preferably constitutive, and optionally regulable promoter, or by the provision of at least a second copy of the AGOS_ACL174W gene (fat1), the AGOS_AER358C gene (pox1), the AGOS_AGL060W gene (fox2), the AGOS_ABL0180 gene (faa1/faa4) and/or the AGOS_AFR302W gene (pot1/fox3) in the genome of the organism. Promoters and methods for the provision of second copies etc. have been described herein above.

In a further aspect the present invention relates to the use of an organism as defined herein above, in particular a genetically modified organisms, e.g. comprising the above mentioned genetic modifications in the fatty acid uptake and/or beta oxidation pathway and optionally further genetic modifications such as modifications to the genes gly1; shm2; ade4; prs 2, 4; prs 3; mls1; bas1; rib 1; rib 2; rib 3; rib 4; rib 5; gua1; ade12; impdh and/or rib 7 as defined herein above, for the production of riboflavin.

In a further aspect the present invention relates to a riboflavin product from at least one organism as defined herein above. The term “riboflavin product from at least one organism as defined herein above” means any product comprising a certain proportion of riboflavin or derivatives thereof which is from any of the inventive organisms as defined herein above. The product may further be a product which has been modified and adjusted in compliance with its purpose. Such products may include edible products suitable as feed for animal or as a human food product, as a dietary supplement or a medical preparation, fungal tablets, food products for babies and children etc. The inventive products may further include riboflavins for industrial production. Further envisaged are riboflavin products useful for chemical synthesis processes, pharmacological purposes and the like.

The following examples and figures are provided for illustrative purposes. It is thus understood that the examples and figures are not to be construed as limiting. The skilled person in the art will clearly be able to envisage further modifications of the principles laid out herein.

### EXAMPLES

### Example 1

### Generation of a FAT1 Over-Expression Construct for the Use in E. Gossypii

For the industrial production of riboflavin the fermentation of E. gossypii was carried out on oil as main carbon source. Riboflavin is then produced from fatty acids through the glyoxylate cycle, gluconeogenesis, the pentose phosphate pathway and the purine and riboflavin synthetic pathways. Therefore, the long-chain fatty acid uptake followed by fatty acid activation as well as the beta-oxidation pathway are the crucial steps to provide acetyl-CoA as one of the precursors necessary for a high riboflavin production.

In E. gossypii, the FAT1 (ACL174W, SEQ ID NO: 2) gene was identified which is the syntenic homolog of the S. cerevisiae Fat1 gene. In S. cerevisiae Fat1p is a bifunctional protein, which plays central roles in fatty acid trafficking at the level of long-chain fatty acid transport and very long-chain fatty acid activation (Zou et al., 2002, Journal of Biological Chemistry, 277, 31062-31071).

In order to evaluate the impact of the fatty acid uptake and activation regarding targeted optimization of the riboflavin biosynthesis in E. gossypii, a construct for the over-expression of the FAT1 gene encoding a fatty acid transporter was generated. For this purpose, the native FAT1 promoter was replaced by the strong and constitutive GPD promoter of E. gossypii.

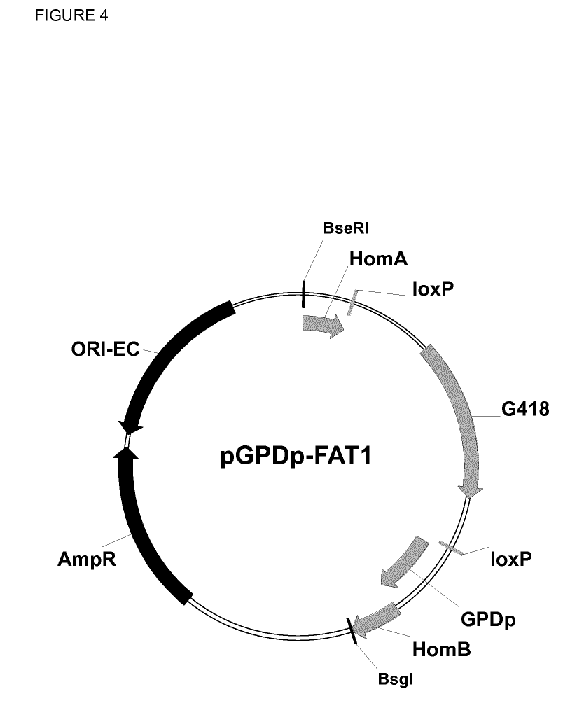

The over-expression plasmid pGPDp-FAT1 (SEQ ID NO: 49, see FIG. 4) for the promoter replacement was assembled via the OPEC cloning method (Quan et al., 2009, PLOS ONE 4: e6441) using four overlapping fragments. All fragments were generated by PCR using specific and overlapping primers. One fragment represents the 3025 bp vector backbone containing the E. coli origin of replication as well as the ampicillin resistance gene for selection in E. coli. Fragments 2 and 3 are the 300 bp or 296 bp long genome integration sites from E. gossypii, so called hom-sites A and B, and the 1959 bp fragment 4 contains the loxP-KanMX-loxP resistance cassette as well as the promoter sequence of the E. gossypii GPD gene.

The repeated inverted loxP sequences enable to eliminate and subsequently reuse the selection marker by expressing a CRE recombinase as described in Guldener et al., 1996, Nucleic Acids Research, 24, 2519-2524.

The resulted plasmid pGPDp-FAT1 was isolated in high amounts using the Plasmid Maxi Kit (Qiagen, Germany). The fragment containing the genome integrations sites A and B, the KanMX resistance marker as well as the GPD promoter sequence was isolated from the plasmid pGPD-FAT1 using BsgI and BseRI digestion. The resulted 2419 bp fragment was gel purified using the Wizard SV Gel and PCR Clean-Up System (Promega, Germany) according to the manufacturer's instructions. The purified fragment was then used for transformation of E. gossypii strain PS3 and the wild type strain ATCC10895. The genomic integration of the over-expression module was confirmed by analytical PCR (see also Example 4 and 7).

### Example 2

### Generation of a POX1 Over-Expression Construct for the Use in E. Gossypii

E. gossypii has one beta-oxidation pathway localized to the peroxisomes (see Vorapreeda et al., 2012, Microbiology, 158, 217-228). According to the Eremothecium/Ashbya Genome Database (http://agd.vital-it.ch/index.html), the genes AER358C (POX1), AGL060W (FOX2) and AFR302W (POT1/FOX3) are syntenic homologs of the S. cerevisiae genes POX1, FOX2 and POT1/FOX3, respectively, which encode the enzymatic activities of the beta-oxidation pathway.

In order to evaluate the impact of the beta-oxidation pathway regarding targeted optimization of the riboflavin biosynthesis in E. gossypii, a construct for the over-expression of the POX1 gene (SEQ ID NO: 6) encoding the acyl-CoA oxidase was generated. For this purpose, the native POX1 promoter was replaced by the strong and constitutive GPD promoter of E. gossypii.

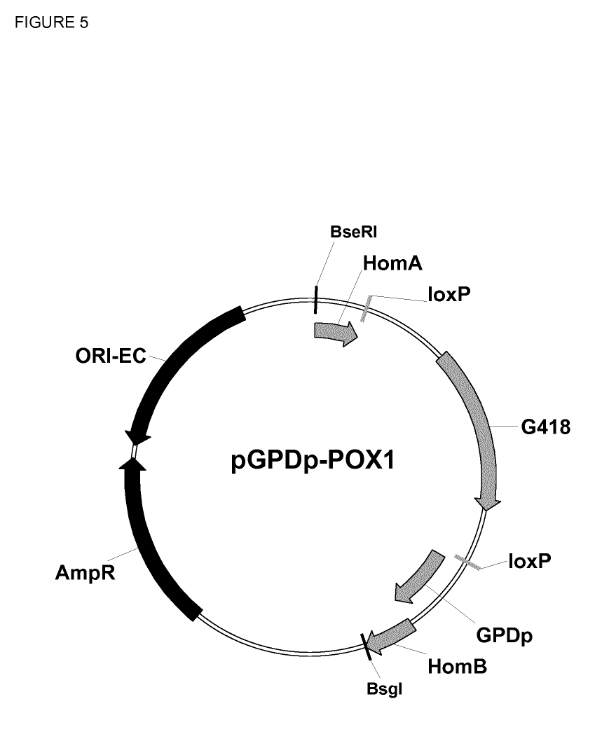

The over-expression plasmid pGPDp-POX1 (SEQ ID NO: 50, see FIG. 5) for the promoter replacement was assembled via the OPEC cloning method (see Quan et al. PLOS ONE 4: e6441) using four overlapping fragments. All fragments were generated by PCR using specific and overlapping primers. One fragment represents the 3026 bp vector backbone containing the E. coli origin of replication as well as the ampicillin resistance gene for selection in E. coli. Fragments 2 and 3 are the 302 bp and 285 bp long genome integration sites from E. gossypii, so called hom-sites A and B, and the 1960 bp fragment 4 contains the loxP-KanMX-loxP resistance cassette as well as the promoter sequence of the E. gossypii GPD gene.

The repeated inverted loxP sequences enable to eliminate and subsequently reuse the selection marker by expressing a CRE recombinase as described in Guldener et al., 1996, Nucleic Acids Research, 24, 2519-2524.

The resulted plasmid pGPDp-POX1 was isolated in high amounts using the Plasmid Maxi Kit (Qiagen, Germany). The fragment containing the genome integrations sites A and B, the KanMX resistance marker as well as the GPD promoter sequence was isolated from the plasmid pGPD-POX1 using BsgI and BseRI digestion. The resulted 2412 bp fragment was gel purified using the Wizard SV Gel and PCR Clean-Up System (Promega, Germany) according to the manufacturer's instructions. The purified fragment was then used for transformation of E. gossypii strain PS3 and the wild type strain ATCC10895. The genomic integration of the over-expression module was confirmed by analytical PCR (see also Example 4 and 7).

### Example 3

### Generation of a POT1-FOX2 Over-Expression Construct for the Use in E. gossypii

E. gossypii has one beta-oxidation pathway localized to the peroxisomes (see Vorapreeda et al., 2012, Microbiology, 158, 217-228). According to the Ashbya Genome Database (http://agd.vital-it.ch/index.html), the genes AER358C (POX1), AGL060W (FOX2) and AFR302W (POT1/FOX3) are syntenic homologs of the S. cerevisiae genes POX1, FOX2 and POT1, respectively, which encode the enzymatic activities of the beta-oxidation pathway.

In order to increase the activity of the beta-oxidation pathway in E. gossypii, a construct for the simultaneous over-expression of the POT1/FOX3 (SEQ ID NO: 10) and FOX2 (SEQ ID NO: 8) genes was generated. POT1 encodes a 3-ketoacyl-CoA thiolase, while the FOX2 protein exhibits hydratase and dehydrogenase activity. For the over-expression, a second copy of both genes was integrated arranged in tandem upstream of the NOP12 (ACR274W) locus in E. gossypii.

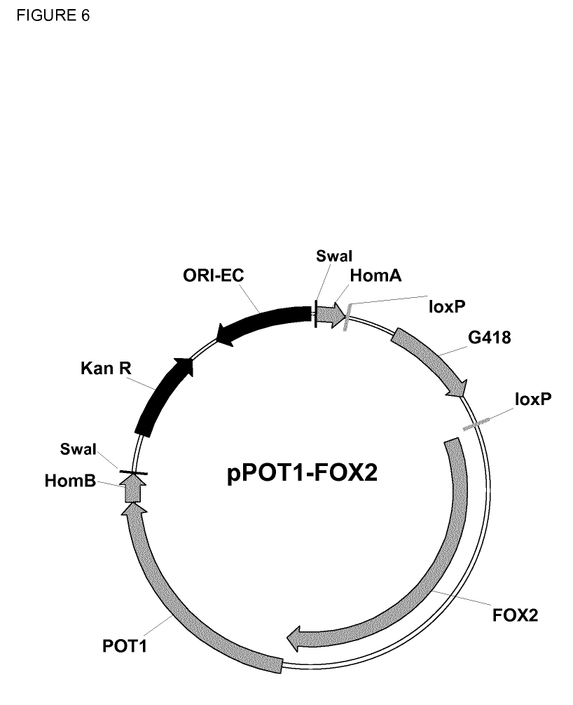

The over-expression plasmid pPOT1-FOX2 (SEQ ID NO: 51, see FIG. 6) was assembled via the OPEC cloning method (see Quan et al. PLOS ONE 4: e6441) using six overlapping fragments. All fragments were generated by PCR using specific and overlapping primers. One fragment represents the 2330 bp vector backbone containing the E. coli origin of replication as well as the kanamycin resistance gene for selection in E. coli. Fragments 2 and 3 are the 296 bp or 297 bp long genome integration sites from E. gossypii, so called hom-sites A and B, and the 1620 bp fragment 4 contains the loxP-KanMX-loxP resistance cassette. Fragment 5 encompasses the POT1 open reading frame together with the promoter and terminator sequences and has a size of 2118 bp. PCR-amplification of the FOX2 gene including the corresponding promoter and terminator results in fragment 6 with a size of 3247 bp.

The repeated inverted loxP sequences enable to eliminate and subsequently reuse the selection marker by expressing a CRE recombinase as described in Guldener et al., 1996, Nucleic Acids Research, 24, 2519-2524.

The resulted plasmid pPOT1-FOX2 was isolated in high amounts using the Plasmid Maxi Kit (Qiagen, Germany). The fragment containing the genome integrations sites A and B, the KanMX resistance marker as well as the POT1 and FOX2 genes was isolated from the plasmid POT1-FOX2 using SwaI digestion. The resulted 7373 bp fragment was gel purified using the Wizard SV Gel and PCR Clean-Up System (Promega, Germany) according to the manufacturer's instructions. The purified fragment was then used for transformation of E. gossypii strain PS3 and the wild type strain ATCC10895. The genomic integration of the over-expression modules was confirmed by analytical PCR (see also Example 4 and 7).

### Example 4

### Generation and Analysis of E. gossypii Strains Over-Expressing Either FAT1, POX1 or POT1 and FOX2

The over-expression cassettes carrying either FAT1 or POX1 under control of the E. gossypii GPD promoter or the second copies of the POT1 and FOX2 genes were constructed and isolated as described above (see also Examples 1 to 3). The purified fragments were transformed using spores of E. gossypii strain PS3 following the protocols provided in Jimenez et al., 2005, Applied Environmental Microbiology 71, 5743-5751. The resulted transformants were selected on MA2 medium (10 g/L Bacto peptone, 10 g/L Glucose, 1 g/L Yeast extract, 0.3 g/L Myoinosit, 20 g/L Agar) containing 200 mg/L Geneticin (G418).

To receive enough mycelium for isolation of genomic DNA the transformants were inoculated on SP medium plates (3 g/L Soybean flour, 3 g/L Yeast extract, 3 g/L Malt extract, 20 g/L Cornmeal, 1 g/L Antifoam, 10 g/1 L Glucose, 30 g/L Agar, pH6.8) containing 200 mg/L Geneticin (G418).

Subsequently, the genomic DNA of each transformant was isolated using the DNeasy Plant Mini Kit (Qiagen, Germany) according to the manufacturer's recommendations. The genomic DNA was then used in different PCR analyses to test the proper integration of the over-expression constructs.

The following PCR analyses were carried out to test the correct integration of the over-expression constructs at the 5′- and 3′ integration sites. A further PCR reaction was done as native control to analyze strain for a homokaryotic background.

| Over-expression | | | |
|---|---|---|---|
| module | 5′ integration site | 3′ integration site | Native control |
| GPDp-FAT1 | P1 (SEQ ID NO: 52) × | P3 (SEQ ID NO: 54) × | P1 (SEQ ID NO: 52) × |
| | P2 (SEQ ID NO: 53) | P4 (SEQ ID NO: 55) | P6 (SEQ ID NO: 56) |
| | → 1244 bp | → 1442 bp | → 736 bp |
| GPDp-POX1 | P7 (SEQ ID NO: 57) × | P9 (SEQ ID NO: 59) × | P7 (SEQ ID NO: 57) × |
| | P8 (SEQ ID NO: 58) | P10 (SEQ ID NO: 60) | P9 (SEQ ID NO: 59) |
| | → 606 bp | → 843 bp | → 796 bp |
| POT1-FOX2 | P11 (SEQ ID NO: 61) × | P12 (SEQ ID NO: 62) × | P11 (SEQ ID NO: 61) × |
| | P8 (SEQ ID NO: 58) | P13 (SEQ ID NO: | P13 (SEQ ID NO: |
| | → 437 bp | 63) | 63) |
| | | → 775 bp | → 478 bp |

Positive transformants were chosen for single spore isolation to be sure to get homokaryotic strains. Single spores were isolated as follow: After dissolving mycelium of transformants in 500 μL Saline-Triton solution (9 g/L NaCl, 600 μl/L Triton X-100) 500 μL of n-hexane was added and mixed. The mixture was centrifuged for 1 min and 14000 rpm and the single spores contained in the upper phase were plated on SP medium plates containing 200 mg/L Geneticin (G418).

Strains resulted from the single spore isolation were tested again using the PCR analysis as described above. Positive strains were used for CRE recombination to eliminate the KanMX selection marker. The transformation for CRE recombination was done as described in Guldener et al., 1996, Nucleic Acids Research, 24, 2519-2524. The resulted strains were PCR analysed to verify the selection marker deletion event. The PCR reactions were carried out as follows:

| Over-expression | |
|---|---|
| module | Selection marker deletion |
| GPDp-FAT1 | P1 (SEQ ID NO: 52) × P15 (SEQ ID NO: 65) |
| | → 896 bp |
| GPDp-POX1 | P16 (SEQ ID NO: 66) × P17 (SEQ ID NO: 67) |
| | → 1017 bp |
| POT1-FOX2 | P11 (SEQ ID NO: 61) × P14 (SEQ ID NO: 64) |
| | → 507 bp |

Strains which have shown the deletion of the selection marker and simultaneously the proper integration of the over-expression modules were chosen for shaking flasks experiments to test the riboflavin production and determine the corresponding yield compared with the reference E. gossypii strain PS3 (see also Example 5).

### Example 5

### Generation and Analysis of E. gossypii Strains Over-Expressing Either FAT1, POX1 or POT1 and FOX2

Since the uptake and activation of fatty acids and an efficient flux through the beta-oxidation pathway are important steps to provide sufficient precursors for the riboflavin production the over-expression of FAT1, POX1, POT1 and FOX2 were carried out. To analyze the effect of gene over-expression on the riboflavin production the above described strains were tested in shaking flask experiments with rapeseed oil as main carbon source and the riboflavin titer was determined. All shaking flask experiments were done in triplicate to evaluate the riboflavin performance of the corresponding strains.

10 ml of pre-culture medium filled in 100 mL Erlenmeyer flasks without baffles was inoculated with E. gossypii mycelium (1 cm²) grown for 3-4 days on SP medium plates. The flasks were incubated for 40 h at 30° C. and 200 rpm. 1 ml of the pre-culture was used to inoculate 24.83 mL main culture medium filled in 250 mL Erlenmeyer flasks with flat baffles. All flasks were weighed to determine the mass before incubation and then incubated for 6 days at 30° C. and 200 rpm. After growth all flasks were weighed again to determine the mass after incubation and therefore to be able to include the evaporation effect during incubation.

| | |
|---|---|
| Pre-culture | 55 g Yeast extract 50 |
| medium | 0.5 g MgSO₄ |
| | → pH7.0 with NaOH |
| | → filled with 950 ml H₂O |
| | 9.5 ml pre-culture medium + 0.5 ml rapeseed oil |
| Main-culture | 30 g Yeast extract 50 |
| medium | 20 g Soybean flour |
| | 10 g Glycine |
| | 7 g Sodium glutamate |
| | 2 g KH₂PO₄ |
| | 0.5 g MgSO₄ |
| | 1.1 g DL-methionine |
| | 0.2 g Inositol |
| | 2.1 g sodium formate |
| | → pH7.0 with NaOH |
| | → filled with 965 ml with H₂O |
| | 21.2 ml main culture medium + 2.8 ml rapeseed oil |
| | → addition of 0.83 ml Urea solution |
| | [15 g Urea/45 ml H₂O] |

The above described cultures were analyzed for riboflavin production using a photometric assay. For this purpose, 250 μL of the culture were mixed with 4.75 mL of 40% solution of nicotinamide and incubated 40 min at 70° C. in darkness. The samples were cooled for 5 min. Subsequently, 40 μL of the samples were mixed with 3 mL H₂O and the extinction at 440 nm was measured. As blank 3 mL H₂O was used. All samples were measured twice.

The riboflavin titer was then calculated according to the following formula:

Titer_{Riboflavin [g/L]}=(Extinction_{[444 nm]}×M_{riboflavin}×nicotinamide dilution×((V_{cuvette}+Vₛₐₘₚₗₑ)/Vₛₐₘₚₗₑ))/molar extinction coefficient/1000

M_{riboflavin}=376,37 mol/L

Molar extinction coefficient=12216L/mol/cm

Formula considering the evaporation during cultivation:

((25,83−(m_{before incubation}−m_{after incubation}))/21,93)×Titer_{riboflavin [g/L]}

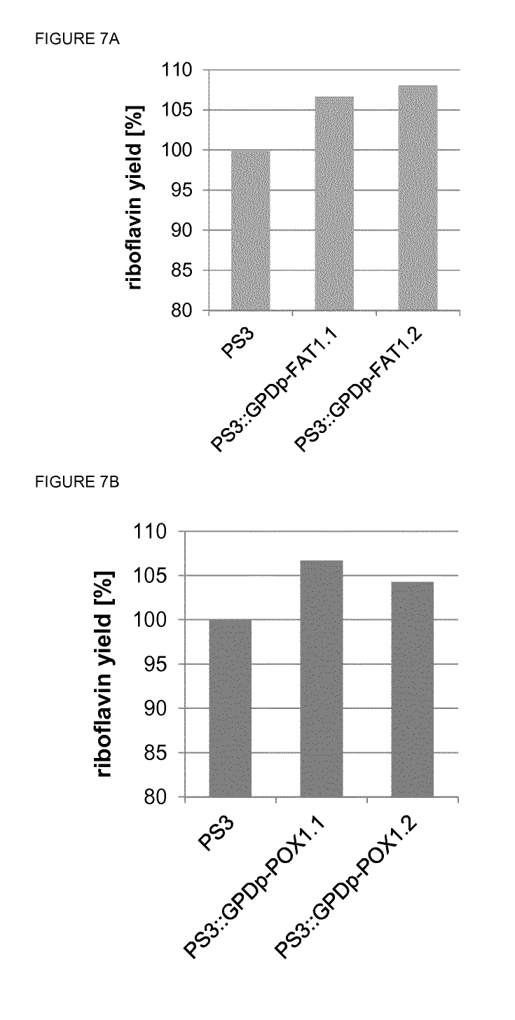

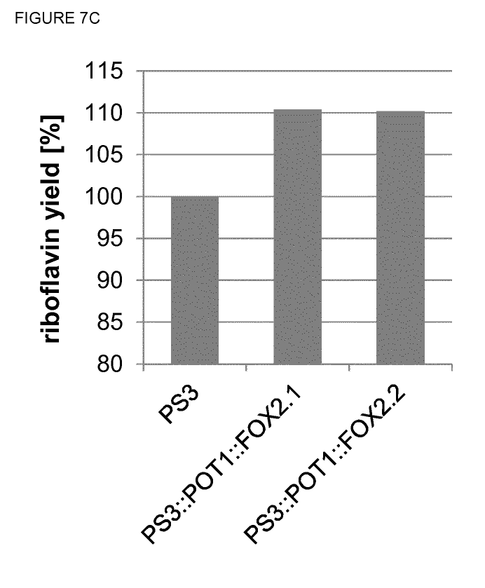

The results as depicted in FIG. 7 show the averaged titer of three independent shake flasks per strain.

Strains over-expressing the FAT1 gene under the GPD promoter of E. gossypii show a 6-8% increase in riboflavin production compared to the reference strain PS3 (see FIG. 7A) concluding that a higher activity of the fatty acid uptake and activation is a key step in the riboflavin production and therefore a suitable target for strain optimization.

Furthermore, it was found that the riboflavin titer was significantly higher in the POX1 as well as in the POT1 and FOX2 over-expression strains than in the reference strain background. Over-expression of POX1 under the GPD promoter leads to an 4-6% increase (see FIG. 7B) while simultaneous over-expression of POT1 and FOX2 bp introduction of a second gene copy results in a 10% higher riboflavin yield (see FIG. 7C).

These results show that the targeted increase of the beta-oxidation pathway activity is an appropriate strategy to significantly improve industrial riboflavin production.

### Example 6

### Generation of a FAA1,4 Over-Expression Construct for the Use in E. gossypii

In E. gossypii, the faa1/faa4 (ABL018C, SEQ ID No. 4) gene was identified which is the syntenic homolog of the S. cerevisiae Faa1 and Faa4 genes. In yeasts fatty acid transport typically requires at least the activities Fat1p, Faa1p and Faa4p. The process of fatty acid transport is apparently driven by the esterifaction of fatty acids as a result of either Faa1p or Faa4p. It is assumed that inter alia Fat1p and Faa1p show functional association and thereby mediate the regulated transport of exogenous long-chain fatty acids.

In order to evaluate the impact of the fatty acid uptake and activation regarding targeted optimization of the riboflavin biosynthesis in E. gossypii, a construct for over-expression of the faa1/faa4 gene encoding a long-chain acyl-CoA synthetase was generated. For the over-expression, a second gene copy was integrated downstream of the MPT5 (ADL056W) locus in E. gossypii.

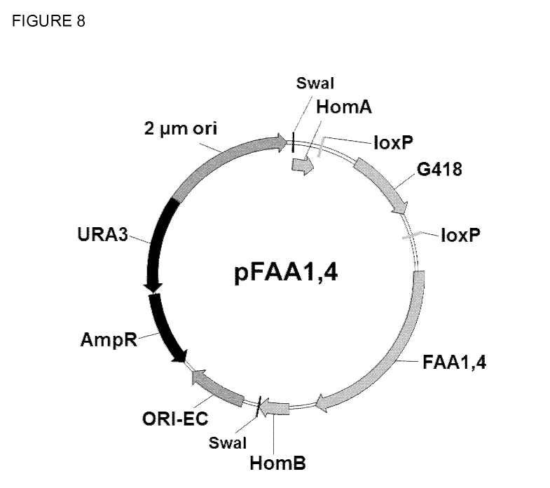

The over-expression plasmid pFAA1,4 (SEQ ID NO: 75, see FIG. 8) was assembled via the transformation-associated recombination cloning in S. cerevisiae (see Kouprina and Larionov, 2008, Nature Protocols 3: 371-377) using seven overlapping fragments. All fragments were generated by PCR using specific and overlapping primers. One fragment represents the 1885 bp vector backbone containing the E. coli origin of replication as well as the ampicillin resistance gene for selection in E. coli. Fragments 2 and 3 are the URA3 gene for selection (1107 bp) and the 2 μm origin (1551 bp) for replication in S. cerevisiae. Fragments 4 and 5 represents the 305 bp or 350 bp long genome integration sites from E. gossypii, so called hom-sites A and B, and the 1581 bp fragment 6 contains the loxP-KanMX-loxP resistance cassette. The repeated inverted loxP sequences enable to eliminate and subsequently reuse the selection marker by expressing a CRE recombinase as described in Guldener et al., 1996, Nucleic Acids Research, 24, 2519-2524. Fragment 7 encompasses the FAA1,4 open reading frame together with the promoter and terminator sequences and has a size of 2757 bp.

All fragments were transformed in S. cerevisiae as described previously (see Kouprina and Larionov, 2008, Nature Protocols 3: 371-377) and the resulting yeast colonies for screened via colony PCR for presence of the corresponding plasmid pFAA1,4. Isolation of the plasmid DNA from a selected positive yeast colony was done using the Wizard Plus SV Minipreps DNA purification kit (Promega, Germany) according to the manufacturer's instructions with the exception of the cell lysis step for which a special yeast cell lysis buffer (0.5 g SDS, 292 mg NaCl, 0.5 ml 1 M Tris/HCl pH8, 1 g Triton X-100 add 50 ml H₂O) was used. The isolated plasmid was then transformed in E. coli for amplification. The resulted plasmid pFAA1,4 was isolated in high amounts using the Plasmid Maxi Kit (Qiagen, Germany).

The fragment containing the genome integrations sites A and B, the KanMX resistance marker as well as the FAA1,4 gene was isolated from the plasmid pFAA1,4 using SwaI digestion. The resulted 5001 bp fragment was gel purified using the Wizard SV Gel and PCR Clean-Up System (Promega, Germany) according to the manufacturer's instructions. The purified fragment was then used for transformation of E. gossypii wild type strain ATCC10895. The genomic integration of the over-expression module was confirmed by analytical PCR (see also Example 7)

### Example 7

### Generation and Analysis of E. gossypii Strains Over-Expressing Either FAT1, POX1, FAA1,4, or POT1 and FOX2 in the Wild Type ATCC10895 Background

The over-expression cassettes carrying either FAT1 or POX1 under control of the E. gossypii GPD promoter or the second copies of the POT1/FOX2 and FAA1,4 genes were constructed and isolated as described above (see also Examples 1 to 3 and Example 6). The purified fragments were transformed using spores of the E. gossypii wild type strain ATCC10895 following the protocols provided in Jimenez et al., 2005, Applied Environmental Microbiology 71, 5743-5751. The resulted transformants were selected on MA2 medium (10 g/L Bacto peptone, 10 g/L Glucose, 1 g/L Yeast extract, 0.3 g/L Myoinosit, 20 g/L Agar) containing 200 mg/L Geneticin (G418).

To receive enough mycelium for isolation of genomic DNA the transformants were inoculated on SP medium plates (3 g/L Soybean flour, 3 g/L Yeast extract, 3 g/L Malt extract, 20 g/L Cornmeal, 1 g/L Antifoam, 10 g/1 L Glucose, 30 g/L Agar, pH6.8) containing 200 mg/L Geneticin (G418).

Subsequently, the genomic DNA of each transformant was isolated using the DNeasy Plant Mini Kit (Qiagen, Germany) according to the manufacturer's recommendations. The genomic DNA was then used in different PCR analyses to test the proper integration of the over-expression constructs.

The following PCR analyses were carried out to test the correct integration of the over-expression constructs at the 5′- and 3′ integration sites. A further PCR reaction was done as native control to analyze strain for a homokaryotic background.

| Over-expression | | | |
|---|---|---|---|
| module | 5′ integration site | 3′ integration site | Native control |
| GPDp-FAT1 | P1 (SEQ ID NO: 52) × | P3 (SEQ ID NO: 54) × | P1 (SEQ ID NO: 52) × |
| | P2 (SEQ ID NO: 53) | P4 (SEQ ID NO: 55) | P6 (SEQ ID NO: 56) |
| | → 1244 bp | → 1442 bp | → 736 bp |
| GPDp-POX1 | P7 (SEQ ID NO: 57) × | P9 (SEQ ID NO: 59) × | P7 (SEQ ID NO: 57) × |
| | P8 (SEQ ID NO: 58) | P10 (SEQ ID NO: 60) | P9 (SEQ ID NO: 59) |
| | → 606 bp | → 843 bp | → 796 bp |
| POT1-FOX2 | P11 (SEQ ID NO: 61) × | P12 (SEQ ID NO: 62) × | P11 (SEQ ID NO: 61) × |
| | P8 (SEQ ID NO: 58) | P13 (SEQ ID NO: | P13 (SEQ ID NO: |
| | → 437 bp | 63) | 63) |
| | | → 775 bp | → 478 bp |
| FAA1,4 | P18 (SEQ ID NO: 76) × | P19(SEQ ID NO: 77) × | P18 (SEQ ID NO: 76) × |
| | P8 (SEQ ID NO: 58) | P20 (SEQ ID | P20 (SEQ ID NO: 78) |
| | → 712 bp | NO: 78) | → 1144 bp |
| | | → 936 bp | |

Positive transformants were chosen for single spore isolation to be sure to get homokaryotic strains. Single spores were isolated as follow: After dissolving mycelium of transformants in 500 μL Saline-Triton solution (9 g/L NaCl, 600 μl/L Triton X-100) 500 μL of n-hexane was added and mixed. The mixture was centrifuged for 1 min and 14000 rpm and the single spores contained in the upper phase were plated on SP medium plates containing 200 mg/L Geneticin (G418).

Strains resulted from the single spore isolation were tested again using the PCR analysis as described above. Positive strains were used for CRE recombination to eliminate the KanMX selection marker. The transformation for CRE recombination was done as described in Guldener et al., 1996, Nucleic Acids Research, 24, 2519-2524. The resulted strains were PCR analysed to verify the selection marker deletion event. The PCR reactions were carried out as follows:

| Over-expression | |
|---|---|
| module | Selection marker deletion |
| GPDp-FAT1 | P1 (SEQ ID NO: 52) × P15 (SEQ ID NO: 65) |
| | → 896 bp |
| GPDp-POX1 | P16 (SEQ ID NO: 66) × P17 (SEQ ID NO: 67) |
| | → 1017 bp |
| POT1-FOX2 | P11 (SEQ ID NO: 61) × P14 (SEQ ID NO: 64) |
| | → 507 bp |
| FAA1,4 | P18 (SEQ ID NO: 76) × P21 (SEQ ID NO: 79) |
| | → 677 bp |

Strains which have shown the deletion of the selection marker and simultaneously the proper integration of the over-expression modules were chosen for shaking flasks experiments to test the riboflavin production and determine the corresponding yield compared with the reference E. gossypii strain ATCC10895 (see also Example 8).

### Example 8

### Analysis of the Riboflavin Production in E. gossypii Strains Over-Expressing Either FAT1, POX1, FAA1/FAA4 or POT1 and FOX2 in the Wild Type ATCC10895 Background

Since the uptake and activation of fatty acids and an efficient flux through the beta-oxidation pathway are important steps to provide sufficient precursors for the riboflavin production the over-expression of FAT1, POX1, FAA1/FAA4, POT1 and FOX2 were carried out. To analyze the effect of gene over-expression on the riboflavin production in the wild type background the above described strains were tested in shaking flask experiments and the riboflavin titer was determined. As reference the parental strain ATCC 10895 was analyzed in parallel.

Total (intracellular and extracellular) riboflavin production levels were measured using a spectrophotometric assay. Strains were cultivated for riboflavin analysis at 28° C. with orbital shaking at 150 rpm in MA2 medium. A volume of 1M HCl was added to 1 mL of culture and incubated at 100° C. for 30 min. After cooling down the samples, the mycelium was lysed using 0.5 mm glass beads (Sigma-Aldrich) and vigorous vortex. After centrifugation, the concentration of riboflavin in the supernatant was determined spectrophotometrically (λexc=450 nm) on a Varioskan microtiter plate reader (Thermo Scientific). The calibration curves were performed using pure riboflavin (Sigma-Aldrich) and processed in the same way as the samples.

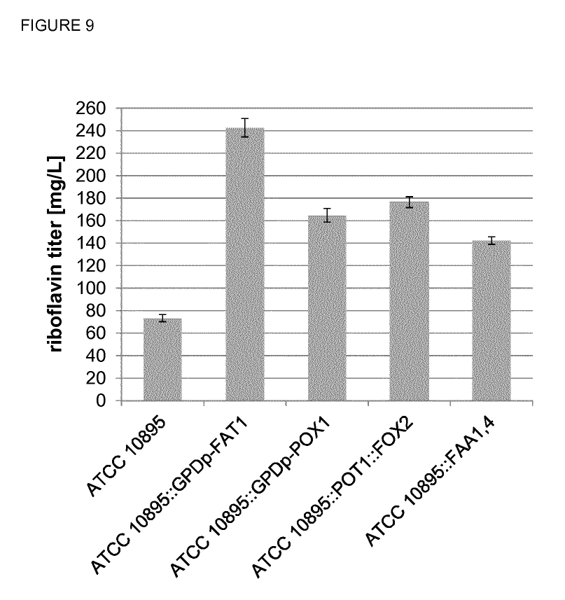

The results as depicted in FIG. 9 show the averaged titer of three independent shake flasks per strain. The wild type strain ATCC10895 shows an average riboflavin titer of about 70 mg/L. An ATCC10895 strain over-expressing the FAT1 gene under the GPD promoter of E. gossypii shows an about 3-fold increase in the riboflavin production compared to the reference strain. Furthermore, over-expression of the FAA1/FAA4 gene results in a 2-fold increase in the riboflavin titer allowing the conclusion that a higher activity of the fatty acid uptake and activation is a key step in the riboflavin production and therefore a suitable target for strain optimization.

In addition, it was found that the riboflavin titer was more than 2-fold higher in the POX1 as well as in the POT1 and FOX2 over-expression strains than in the wild type strain background.

These results show that the targeted increase of the beta-oxidation pathway activity is an appropriate strategy to significantly improve industrial riboflavin production.

1. A method of producing riboflavin in a genetically modified organism of the genus Eremothecium, wherein said genetic modification is linked to the fatty acid uptake and/or beta-oxidation pathway of said organism, comprising:
(i) growing said organisms in a culture medium, preferably in the presence of fatty acid oils; and optionally in the presence of non-lipid carbon sources; and
(ii) isolating riboflavin from the culture medium.

2. A method of providing a riboflavin accumulating organism belonging to the genus Eremothecium by genetically modifying said organism, wherein said genetic modification is linked to the fatty acid uptake and/or beta-oxidation pathway of said organism.

3. A riboflavin accumulating organism belonging to the genus Eremothecium obtained by the method of claim 2.

4. The method of claim 2, wherein said genetic modification results at least in the increase of the AGOS_ACL174Wp (Fat1) activity and/or the increase of the AGOS_AER358Cp (Pox1) activity and/or the increase of the AGOS_AGL060Wp (Fox2) and the AGOS_AFR302Wp (Pot1/Fox3) activity of said organism.

5. The method of claim 2, wherein said genetically modified organism is capable of accumulating at least 5 to 10% more riboflavin than a comparable organism without the genetic modification.

6. The method of claim 4, wherein
(i) said increase of the AGOS_ACL174Wp (Fat1) activity is due to the over-expression of the AGOS_ACL174W gene (fat1); and/or
(ii) said increase of the AGOS_AER358Cp (Pox1) activity is due to the over-expression of the AGOS_AER358C gene (pox1); and/or
(iii) said increase of the AGOS_AGL060Wp (Fox2) activity and the AGOS_AFR302Wp (Pot1/Fox3) activity is due to the over-expression of the AGOS_AGL060W gene (fox2) and the AGOS_AFR302W gene (pot1/fox3).

7. The method of claim 6, wherein said over-expression of the AGOS_ACL174W gene (fat1), the AGOS_AER358C gene (pox1), the AGOS_AGL060W gene (fox2) and/or the AGOS_AFR302W gene (pot1/fox3) is conveyed by a strong, preferably constitutive, and optionally regulable promoter, or by the provision of at least a second copy of the AGOS_ACL174W gene (fat1), the AGOS_AER358C gene (pox1), the AGOS_AGL060W gene (fox2) and/or the AGOS_AFR302W gene (pot1/fox3) in the genome of the organism.

8. The method of claim 2, wherein said genetically modified organism comprises at least one additional genetic modification.

9. The method of claim 8, wherein said additional genetic modification results in the alteration of at least one activity selected from the group consisting of:
(i) GLY1;
(ii) SHM2;
(iii) ADE4;
(iv) PRS 2, 4;
(v) PRS 3;
(vi) MLS1;
(vii) BAS1
(viii) RIB 1;
(ix) RIB 2;
(x) RIB 3;
(xi) RIB 4;
(xii) RIB 5; and
(xiii) RIB 7.

10. The method of claim 8, wherein said additional genetic modification results in at least one of the following alterations:
(i) the GLY1 activity is increased; and/or
(ii) the SHM2 activity is decreased or eliminated; and/or
(iii) the ADE4 activity is increased and/or provided as feedback-inhibition resistant version; and/or
(iv) the PRS 2, 4 activity is increased; and/or
(v) the PRS 3 activity is increased; and/or
(vi) the MLS1 activity is increased; and/or
(vii) the BAS1 activity is decreased or eliminated; and/or
(viii) the RIB 1 activity is increased; and/or
(ix) the RIB 2 activity is increased; and/or
(x) the RIB 3 activity is increased; and/or
(xi) the RIB 4 activity is increased; and/or
(xii) the RIB 5 activity is increased; and/or
(xiii) the RIB 7 activity is increased.

11. A method for increasing the accumulation of riboflavin in an organism of the genus Eremothecium, comprising increasing the activity of the AGOS_ACL174W gene (fat1), the AGOS_AER358C gene (pox1), the AGOS_AGL060W gene (fox2) and/or the AGOS_AFR302W gene (pot1/fox3) by genetic modification.

12. The method of claim 11, wherein the AGOS_ACL174W gene (fat1), the AGOS_AER358C gene (pox1), the AGOS_AGL060W gene (fox2) and/or the AGOS_AFR302W gene (pot1/fox3) is over-expressed via a strong, preferably constitutive, and optionally regulable promoter, or by the provision of at least a second copy of the AGOS_ACL174W gene (fat1), the AGOS_AER358C gene (pox1), the AGOS_AGL060W gene (fox2) and/or the AGOS_AFR302W gene (pot1/fox3) in the genome of the organism.

13. A method for the production of riboflavin, comprising utilizing the organism of claim 3.

14. The method of claim 1, wherein said organism belonging to the genus Eremothecium is of the species Eremothecium ashbyi, Eremothecium coryli, Eremothecium cymbalariae, Eremothecium gossypii, Eremothecium sinecaudum or Eremothecium sp. CID1339.

15. A riboflavin product from the organism of claim 3.

16. The method of claim 1, wherein said genetic modification results at least in the increase of the AGOS_ACL174Wp (Fat1) activity and/or the increase of the AGOS_AER358Cp (Pox1) activity and/or the increase of the AGOS_AGL060Wp (Fox2) and the AGOS_AFR302Wp (Pot1/Fox3) activity of said organism.

17. The method of claim 1, wherein said genetically modified organism comprises at least one additional genetic modification.

18. The method of claim 17, wherein said additional genetic modification results in the alteration of at least one activity selected from the group consisting of:
(i) GLY1;
(ii) SHM2;
(iii) ADE4;
(iv) PRS 2, 4;
(v) PRS 3;
(vi) MLS1;
(vii) BAS1
(viii) RIB 1;
(ix) RIB 2;
(x) RIB 3;
(xi) RIB 4;
(xii) RIB 5; and
(xiii) RIB 7.

19. The method of claim 17, wherein said additional genetic modification results in at least one of the following alterations:
(i) the GLY1 activity is increased; and/or
(ii) the SHM2 activity is decreased or eliminated; and/or
(iii) the ADE4 activity is increased and/or provided as feedback-inhibition resistant version; and/or
(iv) the PRS 2, 4 activity is increased; and/or
(v) the PRS 3 activity is increased; and/or
(vi) the MLS1 activity is increased; and/or
(vii) the BAS1 activity is decreased or eliminated; and/or
(viii) the RIB 1 activity is increased; and/or
(ix) the RIB 2 activity is increased; and/or
(x) the RIB 3 activity is increased; and/or
(xi) the RIB 4 activity is increased; and/or
(xii) the RIB 5 activity is increased; and/or
(xiii) the RIB 7 activity is increased.

20. The method of claim 2, wherein said organism belonging to the genus Eremothecium is of the species Eremothecium ashbyi, Eremothecium coryli, Eremothecium cymbalariae, Eremothecium gossypii, Eremothecium sinecaudum or Eremothecium sp. CID1339.
